(19) United States
(12) Patent Application Publication
(10) Pub. No.: US 2021/0275595 A1
(43) Pub. Date: Sep. 9, 2021

(54) NAÏVE HUMAN EMBRYONIC VASCULAR PROGENITOR CELLS AND METHODS OF TREATMENT

(71) Applicant: The Johns Hopkins University, Baltimore, MD (US)

(72) Inventors: Elias Zambidis, Ellicott City, MD (US); Tea Soon Park, Baltimore, MD (US)

(21) Appl. No.: 17/180,696

(22) Filed: Feb. 19, 2021

Related U.S. Application Data
(60) Provisional application No. 62/979,388, filed on Feb. 20, 2020.

(51) Int. Cl. A61K 35/28; A61P 9/10; A61P 27/02; C12N 5/074; C12N 5/0775

(52) U.S. Cl. CPC A61K 35/28; A61P 9/10; A61P 27/02; C12N 5/0696; C12N 2506/45; C12N 2501/235; C12N 2501/727; C12N 2501/724; C12N 5/0662

(57) ABSTRACT

Compositions are provided compositions comprising tankyrase/PARP (poly-ADP-ribose polymerase, also known as poly-ADP-ribosyltransferase) inhibitor-regulated naïve human induced pluripotent stem cells (N-hiPSCs) and their use in the treatment of vascular disorders.

### CROSS-REFERENCE TO RELATED APPLICATION

This application claims priority to, and the benefit under 35 U.S.C. § 119(e) of U.S. Provisional Application No. 62/979,388, filed Feb. 20, 2020. The entire contents of which are incorporated herein by reference in their entirety.

### STATEMENT REGARDING FEDERALLY SPONSORED RESEARCH

This invention was made with government support under grants EY023962 and HD082098 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 18, 2021, is named 048317-575001US_SL.txt and is 7,541 bytes in size.

### TECHNICAL FIELD

The field of the disclosure relates to vascular regenerative therapies. In particular aspects, the disclosure relates to induced pluripotent stem cells (hiPSC) and human embryonic stem cells (hESC).

### BACKGROUND

The human retina is dependent on an intact, functional vasculature. If either the retinal or choroidal vasculature become compromised, neurons and supporting cells in ischemic areas rapidly die. During progressive diabetic retinopathy (DR), ischemic death of retinal pericytes and endothelial cells (EC) ((Lutty, G. A. Vision Res 139, 161-167, doi:10.1016/j.visres.2017.04.011 (2017); Zheng, L., et al. Invest Ophthalmol Vis Sci 48, 361-367, doi:10.1167/iovs.06-0510 (2007); Joussen, A. M. et al. FASEB J 18, 1450-1452, doi:10.1096/fj.03-1476fje (2004); Joussen, A. M. et al. Am J Pathol 158, 147-152, doi:10.1016/S0002-9440(10)63952-1 (2001)) leads to acellular vascular segments, rapid death of retinal neurons, microglial stimulation, secondary inflammation, macular edema, and subsequent retinal damage from proliferative neovascularization ((D'Amore, P. A. Invest Ophthalmol Vis Sci 35, 3974-3979 (1994); Glaser, B. M., et al. J Cell Biol 84, 298-304, doi:10.1083/jcb.84.2.298 (1980)). If acellular retinal capillaries could be regenerated with patient-specific cellular therapies, neuronal death and pathological neovascularization could be halted or reversed. Human induced pluripotent stem cell (hiPSC) cell therapies offer a versatile patient-specific approach for de novo regeneration of pericytic-EC (Park, T. S. et al. V Circulation 129, 359-372, doi:10.1161/CIRCULATIONAHA.113.003000 (2014); Dar, A. et al. Circulation 125, 87-99, doi:10.1161/CIRCULATIONAHA.111.048264 (2012)). Durable, albeit limited long-term in vivo engraftment of conventional hiPSC-derived vascular progenitor (VP) cells into the ischemic retina was previously reported ((Park, T. S. et al. 2014). However, despite the potential and rapid advance of ocular regenerative medicine ((Mandai, M., et al., N Engl J Med 377, 792-793, doi:10.1056/NEJMc1706274 (2017); Sharma, R. et al. Sci Transl Med 11, doi:10.1126/scitranslmed.aat5580 (2019)), conventional hiPSC lines currently remain limited by highly variable differentiation efficiency and poor in vivo functionality of VP derived from them.

### SUMMARY

We now disclose for the first time advantages of employing an alternative tankyrase/PARP inhibitor-regulated human naïve pluripotent state for improving vascular regenerative therapies. Tankyrase/PARP inhibitor-regulated N-hiPSC represent a new class of human stem cells for regenerative medicine with improved multi-lineage functionality.

Accordingly, embodiments are directed to compositions comprising novel tankyrase/PARP (poly-ADP-ribose polymerase, also known as poly-ADP-ribosyltransferase) inhibitor-regulated naïve human induced pluripotent stem cells (N-hiPSCs) and their use in the treatment of vascular disorders (for example, ischemic retinopathy, neurovascular stroke, and limb ischemia) utilizing naïve embryonic VP differentiated from N-hiPSCs and N-hESCs that possess prolific endothelial-pericytic potential and high epigenetic and developmental plasticity.

Accordingly, in certain embodiments, a method of treating an ischemic organ (e.g. retina) of a subject in need thereof is provided and comprises contacting a human induced pluripotent stem cell (hiPSC) or human embryonic stem cell (hESC) with a composition comprising a leukemia inhibitory factor (LIF) and at least one or more agents which inhibit one or more signaling pathways to produce a naïve human induced pluripotent stem cell (N-hiPSC); administering to the subject, a composition comprising an effective amount of the naïve human induced pluripotent stem cells (N-hiPSC), wherein the N-hiPSC differentiate and revascularize the subject's ischemic retina (or other ischemic organ), thereby treating the ischemic retina (or other ischemic organ).

In additional embodiments, a method of treating an ischemic organ (e.g. retina) of a subject in need thereof is provided and comprises administering to the subject, a composition comprising an effective amount of naïve human induced pluripotent stem cells (N-hiPSC), wherein the N-hiPSC differentiate and revascularize the subject's ischemic retina (or other ischemic organ), thereby treating the ischemic retina (or other ischemic organ), wherein the N-hiPSC are obtainable or obtained from steps comprising, consisting essentially of or consisting of contacting a human induced pluripotent stem cell (hiPSC) or human embryonic stem cell (hESC) with a composition comprising a leukemia inhibitory factor (LIF) and at least one or more agents which inhibit one or more signaling pathways to produce the naïve human induced pluripotent stem cell (N-hiPSC).

In certain embodiments of the present methods and compositions, the one or more agents comprise inhibitors of tankyrase or PARP, mitogen-activated protein kinase kinase (MEK), Glycogen Synthase Kinase 3-β (GSK3β) or signaling pathways thereof. In certain embodiments a tankyrase/PARP inhibitor comprises: XAV939, IWR-1, G007-LK, JW55, AZ1366, JW 74, NVP-TNKS656 or combinations thereof. In certain embodiments, a GSK30 inhibitor comprises: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR 99021), 5-Ethyl-7,8-dimethoxy-1H-pyrrolo[3,4-c]isoquinoline-1,3(2H)-dione (3F8), 1-(7-Methoxyquinolin-4-yl)-3-[6-(trifluoromethyl)pyridin-2-yl]urea (A 1070722), N6-[2-[[4-(2,4-Dichlorophenyl)-5-(1H-imidazol-1-yl)-2-pyrimidinyl]amino]ethyl]-3-nitro-2,6-pyridinediamine (CHIR 98014), lithium chloride (LiCl), 4-benzyl-2-methyl-1,2,4-thiadiazolidine-3,5-dione (TDZD-8), 5-iodo-indirubin-3′-monoxime (I3′M) and N-(4-methoxybenzyl)-N′-(5-nitro-1,3-thiazol-2-yl)urea (AR-A014418) or combinations thereof. In certain embodiments, an MEK inhibitor comprises: PD032590, CI-1040 (PD184352), cobimetinib (GDC-0973, XL518), Selumetinib (AZD6244), MEK162, AZD8330, TAK-733, GDC-0623, Refametinib (RDEA119; BAY 869766), Pimasertib (AS703026), RO4987655 (CH4987655), RO5126766, WX-554, HL-085 or combinations thereof. In certain embodiments, the hiPSCs are derived from primed isogenic hiPSCs. In certain embodiments, the hiPSC are derived from diabetic donor hiPSCs (DhiPSC) or non-diabetic donor hiPSCs.

In certain embodiments, a method of producing a vascular progenitor (VP) cell with high developmental and epigenetic plasticity and with prolific endothelial-pericytic potential comprises contacting a human induced pluripotent stem cell (hiPSC) with a composition comprising a leukemia inhibitory factor (LIF) and at least one agent or a combination of at least three agents which inhibit one or more signaling pathways to produce a naïve human induced pluripotent stem cell (N-hiPSC); and, differentiating the N-hiPSC in vitro or by implantation in vivo. In certain embodiments, the hiPSCs are derived from fibroblasts, cord blood cells, human adult or fetal stem cells, bone marrow cells, human induced pluripotent stem cell lines or combinations thereof. In certain embodiments, the hiPSC are derived from diabetic donor hiPSCs (DhiPSC) or non-diabetic donor hiPSCs. In certain embodiments, the hiPSCs are autologous, isogenic, allogeneic, haplotype matched, haplotype mismatched, haplo-identical, xenogeneic or cell lines. In certain embodiments, the at least one agent is an inhibitor of poly-ADP-ribosyltransferase and signaling pathways thereof. In certain embodiments, the at least one agent is an inhibitor of mitogen-activated protein kinase kinase (MEK) and signaling pathways thereof. In certain embodiments, the at least one agent is an inhibitor of Glycogen Synthase Kinase 3 (GSK3) or signaling pathways thereof.

In certain embodiments, the composition comprises a leukemia inhibitory factor (LIF) and a combination of at least three agents comprising inhibitors of poly-ADP-ribosyltransferase (also known as as poly-ADP-ribose polymerases; PARP), MEK, GSK3 and signaling pathways thereof. In certain embodiments, the poly-ADP-ribosyltransferase is tankyrase. In certain embodiments, the GSK3 is a GSK3β isoform. In certain embodiments, a tankyrase inhibitor comprises: XAV939, IWR-1, G007-LK, JW55, AZ1366, JW 74, NVP-TNKS656 or combinations thereof. In certain embodiments, a GSK3β inhibitor comprises: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR 99021), 5-Ethyl-7,8-dimethoxy-1H-pyrrolo[3,4-c]isoquinoline-1,3(2H)-dione (3F8), 1-(7-Methoxyquinolin-4-yl)-3-[6-(trifluoromethyl)pyridin-2-yl]urea (A 1070722), N6-[2-[[4-(2,4-Dichlorophenyl)-5-(1H-imidazol-1-yl)-2-pyrimidinyl]amino]ethyl]-3-nitro-2,6-pyridinediamine (CHIR 98014), lithium chloride (LiCl), 4-benzyl-2-methyl-1,2,4-thiadiazolidine-3,5-dione (TDZD-8), 5-iodo-indirubin-3′-monoxime (13′M) and N-(4-methoxybenzyl)-N′-(5-nitro-1,3-thiazol-2-yl)urea (AR-A014418) or combinations thereof. In certain embodiments, an MEK inhibitor comprises: PD032590, CI-1040 (PD184352), cobimetinib (GDC-0973, XL518), Selumetinib (AZD6244), MEK162, AZD8330, TAK-733, GDC-0623, Refametinib (RDEA119; BAY 869766), Pimasertib (AS703026), RO4987655 (CH4987655), RO5126766, WX-554, HL-085 or combinations thereof.

In certain embodiments, a combination of MEK and GSK3β inhibitors comprises PD0325901 plus CHIR99021.

In certain embodiments, a composition comprises a tankyrase inhibitor, a MEK inhibitor and a GSK3β inhibitors. In certain embodiments, the tankyrase inhibitor is XAV939. In certain embodiments, the MEK inhibitor is PD032590. In certain embodiments the GSK30 inhibitor is CHIR9902. In certain embodiments the composition comprises XAV939, PD0325901 and CHIR99021.

In certain embodiments, a method of reverting a primed human induced pluripotent stem cell (hiPSC) to a naïve hiPSC, comprises contacting a human induced pluripotent stem cell (hiPSC) with a composition comprising a leukemia inhibitory factor (LIF) and at least three agents which inhibit one or more signaling pathways to produce a naïve human induced pluripotent stem cell (N-hiPSC). In certain embodiments, the agents comprise inhibitors of tankyrase, mitogen-activated protein kinase kinase (MEK), Glycogen Synthase Kinase 3-β (GSK3β) or signaling pathways thereof. In certain embodiments, the hiPSCs are derived from primed isogenic hiPSCs.

In certain embodiments, a composition comprises an effective amount of naïve human induced pluripotent stem cells (N-hiPSCs) wherein the N-hiPSCs are tankyrase inhibitor regulated. In certain embodiments, the hiPSC is reprogrammed from donor diabetic or donor non-diabetic fibroblasts.

In certain embodiments, a method of treating disorders of a subject's vascular system is provided and comprises contacting a human induced pluripotent stem cell (hiPSC) with a composition comprising a leukemia inhibitory factor (LIF) and at least one or more agents which inhibit one or more signaling pathways to produce a naïve human induced pluripotent stem cell (N-hiPSC); administering to the subject, a composition comprising an effective amount of the naïve human induced pluripotent stem cells (N-hiPSC), wherein the N-hiPSC differentiate and revascularize the subject's vascular system, thereby treating the vascular disorder.

In additional aspects, a method of treating disorders of a subject's vascular system is provided and comprises administering to the subject, a composition comprising an effective amount of the naïve human induced pluripotent stem cells (N-hiPSC), wherein the N-hiPSC differentiate and revascularize the subject's vascular system, thereby treating the vascular disorder, wherein the N-hiPSC are obtainable or obtained from steps comprising, consisting essentially of or consisting of contacting a human induced pluripotent stem cell (hiPSC) with a composition comprising a leukemia inhibitory factor (LIF) and at least one or more agents which inhibit one or more signaling pathways to produce the N-hiPSC.

Any compositions or methods provided herein can be combined with one or more of any of the other compositions and methods provided herein.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

An “effective amount” as used herein, means an amount which provides a therapeutic or prophylactic benefit.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

“Vascular progenitor cells” are progenitors for pericyte stem cells. Pericytes are multi-functional cells embedded within the walls of capillaries throughout the body, including the brain. Pericytes are a major therapeutic category of stem cells with broad interest to regenerative medicine. Vascular progenitor cells are the precursors of endothelial and perivascular cells, the latter include smooth muscle cells and multipotent pericytes. Various native tissues, as well as human pluripotent stem cells, either embryonic or induced, have been shown to provide a plentiful source of vascular progenitor cells and their derivatives. These progenitor cells and derivatives can potentially be applied to the repair of ischemic tissues and the vascularization of engineered bio-constructs, as well as the regeneration of heart, muscle, cartilage and bone.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D are a series of images and graphs demonstrating the multi-lineage teratoma organoid quantifications in isogenic primed vs. naïve non-diabetic hiPSC. The normal, non-diabetic human fibroblast-hiPSC line C1.2 was cultured in parallel in either primed, conventional E8 (PRIMED; P) or LIF-3i/MEF naïve (NAÏVE; N) conditions prior to parallel injections into sibling NOG mice (5×10⁶ cells/site) for teratoma assays. Paraffin sections of 8-week-old N vs P teratomas were evaluated and individual microscopic teratoma sections quantified by (FIGS. 1A, 1B) H&E staining, or (FIGS. 1C, 1D) immunofluorescence (IF) staining. Shown are individual tissue section measurements from at least 3 independent teratoma experiments quantified for organoid structures and markers of endodermal (Cytokeratin 8⁺ (CK8); gut/glandular structures), mesodermal (NG2⁺ chondroblasts), and ectodermal (SOX2⁺ neural rosettes) lineages along with the proliferation marker Ki-67, as described in Methods. Scale Bar=50 μm. **=p<0.01; ***=p<0.001 (Mann-Whitney tests).

FIGS. 2A-2E are a series of immunofluorescent stain, blots and a graph demonstrating the generation of isogenic conventional and naïve DhiPSC lines. FIG. 2A: Immunofluorescent stains of N-DhiPSC (line E1C1) following LIF-3i reversion from its conventional primed state for general pluripotency factors (TRA-1-81, NANOG, OCT4) and naïve pluripotency proteins (KLF2, NR5A2, TFCP2L1, STELLA/DPPA3, E-CADHERIN; Scale Bar=50 μm). Primed- vs. naïve-cultured isogenic hPSC line protein lysates were prepared from 3 independent DhiPSC lines (E1C1, E1CA1, E1CA2), an hESC line (H9), and normal non-diabetic fibroblast-hiPSC lines (C1.2, C2). Western blots were performed of primed (P) vs naïve (N) lysates of isogenic DhiPSC line E1C1, normal CB-iPSC line E5C3, and hESC line H9, with ACTIN or total STAT3 serving as internal loading controls for each blot. FIG. 2B: Expressions of phosphorylated (P-STAT3) and total STAT3 (T-STAT3; control) in isogenic primed (P) vs naïve (N) conditions from (upper panel) 3 independent DhiPSC lines (E1C1, E1CA1, E1CA2, and (lower panel) 2 independent, isogenic normal non-diabetic fibroblast-hiPSC lines (C1.2, C2) in primed vs naïve conditions. FIG. 2C: Naïve-specific expression of pluripotency factor TFAP2C is shown for DhiPSC line E1C1 in primed (P) vs naïve (N) isogenic conditions. FIG. 2D: Isogenic teratoma organoid quantifications from diabetic hiPSC line E1C1 cultured in primed (blue bar), vs naïve (red bar) conditions. Shown are quantifications per cross section of mesodermal (NG2⁺ chondroblast), definitive endodermal (CK8⁺ gut/glandular cells), and ectodermal (SOX2+ neural rosettes; retinal pigmented epithelium) structures from H&E stained slides. **=p<0.01; * **=p<0.001 (Mann-Whitney tests). FIG. 2E: Western blot analysis of XAV939-inhibited proteolysis of tankyrases 1 and 2 (TANK ½) and AXIN-1 proteins from DhiPSC line E1C1, CB-hiPSC line E5C3, and hESC line H9, in isogenic naïve (N) vs. primed (P) conditions.

FIGS. 3A-3D are a series of schematics, graphs and images demonstrating vascular differentiations of primed vs. naïve non-diabetic and diabetic hiPSC. FIG. 3A: (Upper panel) schematic of experimental design of isogenic primed (E8) vs naïve (LIF-3i) sibling DhiPSC, differentiated in parallel following 5-7 passages in their respective primed vs naïve culture conditions, as described in Methods. (Lower panel) defined xeno-free APEL vascular differentiation system. Shown are indicated growth factors and inhibitor molecules, as described in Methods. Day 7 differentiation cultures were enriched for CD31-expressing VP using magnetic-activated cell sorting (MACS); CD31⁺ VP co-express CD146 post-MACS enrichment. These CD31⁺CD146⁺ VP populations were further expanded for several passages in EGM2 medium prior to in vitro characterization or injection into I/R-injured murine NOG eyes. FIG. 3B: Kinetics of surface protein expressions by flow cytometry during APEL vascular differentiation for pluripotency markers (SSEA4, TRA-1-81) and vascular markers (CD31, CD146, CD34, CD144) from isogenic primed (blue) and naïve (red) DhiPSC lines. Shown are mean results with SEM of two independent differentiation experiments of N-DhiPSC and their isogenic primed DhiPSC counterparts (lines E1C1, E1CA1; n=2). FIG. 3C: Average percentages of CD31⁺CD146⁺ cells obtained from APEL differentiation cultures of isogenic independent non-diabetic CB-iPSC and diabetic hiPSC lines on differentiation days 7-8 (i.e., day of CD31⁺ sorting (see scheme above). Results of independent experiments are shown for differentiation of the non-diabetic CB-iPSC line E5C3 and two DhiPSC lines (E1CA1, E1CA2) starting from simultaneous and isogenic primed and naïve cultures. *=p<0.05 (unpaired two-tailed t tests). FIG. 3D: Transmission electron microscopy (TEM) images of primed DVP and N-DVP differentiated and expanded as described above from parallel primed and naïve isogenic conditions of the DhiPSC line E1C1. WPB: Weibel-Palade body, n: nucleus, TEC: transcytotic endothelial channel; Scale Bar=400 nm.

FIGS. 4A-4D are a series of plots, graphs and an image showing the characterization of DVP generated from primed vs. naïve DhiPSC. FIG. 4A: Endothelial functionality. Shown are representative flow cytometry (left panel) and immunofluorescent Dil-acetylated-LDL (Dil-Ac-LDL) endothelial uptake assays (right panel); merged phase contrast/Ac-Dil-LDL-labeled primed DVP vs. N-DVP cells; Scale Bar=100 μm. DVP cells were generated from primed vs naïve isogenic DhiPSC line E1CA2. FIG. 4B: EdU proliferation assays of purified DVP after 4 passages in EGM2 post-CD31⁺ purification. DhiPSC line E1CA2; n=2 independent experiments. FIG. 4C: Expanded VP and N-VP were quantitated for senescent cells by β-galactosidase activity colorimetric assay. Shown are isogenic independent comparisons of both non-diabetic primed VP and N-VP (i.e., generated from H9 hESC, C1.2, C2 fibroblast-hiPSC lines) and diabetic DVP and N-DVP (i.e., generated from E1CA1, E1CA2, E1C1 DhiPSC lines). Each quantitation is an independent measurement of EGM2 cultures at indicated matched passages for each VP and N-VP type. ***=p<0.001 (multiple unpaired t tests). FIG. 4D: Quantification of vascular tube lengths formed from in vitro Matrigel tube assays from primed normal VP (E5C3) and isogenic primed DVP vs N-DVP (line E1CA2). The number (n) of total measurements of each of the three experimental groups from 3-5 independent experiments per group is labeled. *=p<0.05 (unpaired t tests).

FIGS. 5A-5C are a series of images, a graph and a blot demonstrating the DNA damage responses in primed DVP vs. N-DVP. FIG. 5A: Purified and expanded CD31⁺ DVP or N-DVP (line E1CA2) were treated with the radiomimetic drug NCS for 5 hours before fixation and staining with antibodies for detection of human CD31⁺ cells and phosphorylated H2AX (pH2AX) positive nuclear foci (i.e., DAPI co-staining) to reveal double-strand DNA breaks (arrows); Scale Bar=50 μm. FIG. 5B: Quantification of pH2AX foci per nuclei in isogenic primed vs naïve DVP with or without induction of DNA damage with NCS. Shown are numbers of DAPI⁺ nuclei per field with no pH2AX foci (green), and DAPI+ nuclei with 1-5 foci (light pink), 6-10 (dark pink) and >10 pH2AX foci (red). ***=p<0.0001; Chi-Square tests. FIG. 5C: Lysates of primed DVP and and N-DVP (line E1C1) cultured in EGM2 and treated with and without NCS for 5 hours were analyzed by Western blotting for expressions of proteins activated by DNA damage and apoptosis (i.e., total H2AX and phosphorylated H2AX (P-H2AX), RAD51, RAD54, phosphorylated p53 (P-p53), total DNA-PK and phosphorylated DNA-PK (P-DNA-PK).

FIGS. 6A-6F are a series of schematics and images demonstrating the efficient survival and human vascular engraftment of N-DVP in I/R-injured murine retinae. FIG. 6A: Schematic of NOG mouse ocular I/R experimental system for testing in vivo functionality of human primed DVP vs N-DVP. Shown are anatomical structures where I/R (anterior chamber) and human DVP and N-DVP cell injections (vitreous body) were performed (left panel). Schematic of timeline for I/R injury surgery, human cell DVP injections (Day 0), and days of analysis of human cell survival and engraftment (right panel). FIG. 6B: Human DVP cell survival at the superficial layer of murine retina at three weeks following injection of 50,000 DVP or N-DVP into the vitreous of I/R-treated NOG mouse eyes. Flat whole-mounted retinae were stained with antibodies for human-specific HNA (red), and tile scanned by confocal microscopic imaging (10× objective, 9×9 tiles). Shown are representative whole retinal images with HNA⁺ cells from primed DVP cell-injected (left panel) vs. N-DVP cell-injected (right panel) eyes. Scale bars=500 μm. FIG. 6C: Quantitation of HNA⁺ cells detected in the outer superficial layers of whole mount retinae following treatment of eyes with and without I/R, and injected with either primed DVP or N-DVP at (FIG. 6C) 4 weeks or (FIG. 6D) at 1, 3, and 4 weeks following DVP vs. N-DVP vs. control saline (PBS) injections, in eyes treated with and without I/R injury. Shown are the mean numbers from independent eye experiments of total HNA⁺ cells counted with imaging software per superficial layer of each whole-mounted retinae (whole field). **=p<0.01 (Mann-Whitney tests). FIG. 6E: Human vascular engraftment. Whole-mounted retinae of I/R-injured eyes of NOG mice 2 weeks following DVP vs. N-DVP vs. PBS injections into ischemia-injured eyes were immuno-stained with human CD34 (hCD34) to detect human endothelial engraftment. Antibodies for murine collagen type-IV (mCol-IV) were also employed (to detect murine blood vessel basement membrane), and murine CD31 (mCD31) (to detect murine endothelium). FIG. 6F: The number of CD34+ human-murine chimeric vessels per 450 μm cross-section were quantitated via confocal microscopy and imaging software. Shown are results of independent measurements. ***=p<0.001 (multiple unpaired t tests).

FIGS. 7A-7D are a series of images and graphs demonstrating the migration of primed DVP and N-DVP into ischemia-injured blood vessels and vascular engraftment in the neural retina. Whole mount retinae of/R-injured eyes of NOG mice were immuno-stained with either human CD34 (FIGS. 7A, 7B; hCD34) or human CD31 (FIGS. 7C, 7D; hCD31) antibodies to detect human endothelial cells 2 weeks following DVP vs. N-DVP injections. Antibodies for murine collagen type-IV (mCol-IV) were also employed to detect murine blood vessel basement membrane, and murine CD31 (mCD31) detected murine endothelium. The number of human CD34+(FIG. 7B) or (FIG. 7D) human CD31V cells detected within transverse layers of the murine neural retina (per 450 μm retinal cross section; see FIGS. 16A, 16B) was quantitated. Each data point represents a replicate individual 450 μm retinal cross section that was analyzed from I/R-treated eyes injected with saline (PBS), primed DVP or N-DVP. Human CD34+ or human CD31V endothelial cell engraftment was enumerated in each distinct layer of neural retina shown, and demonstrated that only N-DVP migrated into the inner nuclear layer (INL) while most of the primed DVP remained primarily in the superficial ganglion cell layer (GCL). ILM: inner limiting membrane, IPL: inner plexiform layer, outer nuclear layer (ONL), OPL: outer plexiform layer, S: segments. All scale bars=50 μm.

FIGS. 8A-8E are a series of graphs and blots demonstrating the epigenetic configurations of multi-lineage bivalent and vascular lineage-specific promoters in primed vs naïve hiPSC and VP. FIG. 8A: Densitometric quantitation of dot immuno-blots of global levels of 5-methylcytosine (5mC) and 5-hydroxymethylcytosine (5mC) in three isogenic pairs of primed (E8) and naïve (MEF-depleted LIF-3i) DhiPSC lines. Genomic DNA samples were collected from parallel isogenic primed (E8 medium) and naïve LIF-3i) cultures. Immunoblot densities LIF-3i/E8 ratios were determined with ImageJ software at steady state conditions (200 ng), and normalized at 100% for E8 values. FIG. 8B: Western blot analysis of PRC2 components EZH1, EZH2, SUZ12, and JARID2) in primed vs naïve normal and diabetic hiPSC lysates; E1C1 fibroblast-DhiPSC line; C1.2 normal donor fibroblast-hiPSC line. FIG. 8C: ChIP-qPCR for H3K27me3 and H3K4me3 histone marks at key known bivalent developmental promoters in primed vs naïve DhiPSC (e.g., PAX6, MSX2, GATA6, SOX1, HAND1, GATA2). Levels of GAPDH and NANOG are controls for actively transcribed genes. Data is presented as differences in percent input materials of naïve minus primed genomic DNA samples for DhiPSC line E1C1. Bars represent the SEM of replicates. FIG. 8D: ChIP-qPCR for H3K27me3 and H3K4me3 histone marks at key vascular developmental promoters in primed vs naïve VP genomic samples. Data is presented as GAPDH-normalized ratios of percent input materials between naïve and primed VP differentiated from the DhiPSC line E1C1. Results are shown as ratios of expression of isogenic N-DVP vs. DVP for GATA2-regulated genes (CD31, vWF, endothelin-1, ICAM2) and genes regulated by histone marks that are known to effect vascular functionality (CXCR4, DLL1, FZD7). The histone profile for GAPDH is a ‘housekeeping control gene. NANOG and MYOD1 are control gene promoters that become repressed during vascular differentiation. FIG. 8E: qRT-PCR gene expression analysis of vascular lineage genes (left panel) and PRC2-regulated bivalent lineage-specific genes (right panel) in DVP vs. N-DVP that were differentiated from isogenic pairs of naive vs primed D-hiPSC lines (n=3; lines E1C1, E1CA1, E1CA2). Fold changes are normalized to beta-actin expression. All PCR primers are listed in Table 1 below.

FIGS. 9A-9B are schematics showing models for tankyrase inhibitor-mediated reversion of conventional primed hiPSC to an epigenetically plastic naive pluripotent state. FIG. 9A shows the epigenetic obstacles of incomplete reprogramming, lineage priming, and disease-associated epigenetic aberrations in conventional primed hiPSC (blue) can be overcome with molecular reversion to a tankyrase inhibitor-regulated naïve epiblast-like state (red) with a more primitive, unbiased epigenetic configuration. FIG. 9: Compared to conventional lineage primed DhiPSC, tankyrase-inhibited N-DhiPSC possessed a de-repressed naïve epiblast-like epigenetic configuration at bivalent PRC2-regulated developmental promoters that was highly poised for non-biased, multi-lineage lineage specification.

FIGS. 10A-10E are a series of images and plots demonstrating the in vitro multi-lineage directed differentiations of paired isogenic primed vs. naïve normal (non-diabetic) hiPSC lines. Isogenic primed (E8 cultures) vs. naïve-reverted (LIF-3i cultures) hiPSC were differentiated in parallel using established multi-lineage protocols and commercially available kits. Shown are direct comparisons between normal (non-diabetic) isogenic hiPSC lines demonstrating augmented differentiation capacities to all three germ layers and robust/improved capacity for terminal differentiation. FIG. 10A: Neuro-ectodermal differentiation of isogenic comparisons of primed vs. naïve hPSC lines performed as described with PSC neural induction medium (Thermo Fisher Scientific; n=6, three normal fibroblast-hiPSC lines (circles; C1.2, C2, 7ta) and three normal cord blood (CB)-derived hiPSC lines (triangles; E5C3, E5C1, LZ610). Shown are flow cytometry analysis of differentiation cultures for protein expressions of % Nestin⁺SOX1⁺ neural progenitor cells determined by flow cytometry. FIG. 10B: Representative images of Tuj1-expressing terminally-differentiated cells from a primed (E8 culture) vs naïve (LIF-3i culture) CB-iPSC line (E5C3) after 5 weeks of neural differentiation, performed, and demonstrating higher incidence of extended Tuj1⁺ neurites elongated over 3 mm in N-CB-iPSC relative to primed CB-iPSC. FIG. 10C: Definitive endodermal differentiation of isogenic primed vs naïve-hiPSC (n=6), each individual line represented by a different color, with mean and SEM shown). Differentiations were performed as described with STEMdiff definitive endoderm kit and STEMdiff APEL medium (StemCell Technologies). Shown are % differentiated cells expressing FOXA2⁺ endodermal progenitor cells. FIGS. 10D, 10E: Hemato-vascular (mesodermal) differentiations of non-diabetic isogenic primed vs naïve hiPSC lines were performed at day 10 of embryoid body differentiation cultures for (FIG. 10D) the hESC line RUES02, and (FIG. 10E). Shown are % cells in differentiation cultures expressing surface markers for endothelial-vascular progenitors (e.g., CD31⁺, CD34⁺, CD144⁺, CD31⁺CD146⁺, KDR⁺), pericytes (e.g., CD140b⁺, CD90⁺NG2⁺), angioblasts (e.g., CD105⁺, CD143⁺), and also non-vascular lineage markers (e.g., CD15⁺). *=p<0.05; **=p<0.01 (two-tailed unpaired t tests).

FIGS. 11A-11F are a series of schematics and images demonstrating the non-integrated episomal reprogramming of type I diabetic skin fibroblasts into conventional DhiPSC lines, and subsequent naïve reversion into N-DhiPSC with the LIF-3i culture system. FIG. 11A: Scheme of timeline of reprogramming of diabetic skin fibroblasts for the generation of conventional, primed DhiPSC. FGM: fibroblast growth medium, ES: ES medium, AA: ascorbic acid, CHIR99021: GSK-β inhibitor, E8: essential 8 medium. FIG. 11B (panel a) shows the typical morphology of conventional primed DhiPSC cultured in E8 medium on vitronectin-coated plates. FIG. 11B (panel b) shows the teratoma formation in NOG mice from primed DhiPSC line (E1CA1) generated well-developed three germ layer organoid structures. FIG. 11C: Flow cytometry analysis of conventional DhiPSC lines demonstrated >95% SSEA4⁺Tra1-81⁺ expressions. FIG. 11D: Scheme of timeline of naïve reversion (LIF-5i/LIF-3i) of primed, conventional DhiPSC into N-DhiPSC. FIG. 11E shows the typical dome-shaped colonies (left, panels a and c) in LIF-3i naïve cultures of N-DhiPSC lines (E1C1, E1CA2) and their normal G-banded karyotypes (right, panels b and d) following 3-10 passages in naïve (LIF-3i) conditions. FIG. 11F: Representative teratoma H&E sections from two N-DhiPSC lines (E1CA2 and E1C1). Shown are ectodermal neural rosette (Ect), endodermal epithelial gut (End), and mesodermal cartilage (Meso) structures. Scale bar=100 μm.

FIGS. 12A-12D are a series of images and graphs demonstrating the APEL vascular differentiation of primed and naïve DhiPSC. FIG. 12A: Both primed (E8) and naïve (LIF-3i) DhiPSC (line E1C1) differentiated efficiently using the APEL monolayer vascular differentiation system. Shown are morphologies of APEL-differentiated DVP cells before and after CD31-sorting and re-plating in EGM2. FIG. 12B: Representative flow cytometry expressions of CD31 and CD146 prior to and post sorting of APEL differentiation cultures with CD31 magnetic bead-tagged antibody (MACS: magnetic activated cell sorting). FIG. 12C: Post CD31-sort (prior to EGM2 expansion) vascular lineage surface marker analysis of primed DVP (left panel) vs N-DVP (right panel) demonstrating no significant differences in vascular marker expressions post CD31-sorting from APEL cultures. FIG. 12D: CD31⁺CD146⁺ N-VP and N-DVP were generated from normal non-diabetic naïve N-CBiPSC and naïve N-DhiPSC, respectively, and analyzed post-CD31 sorting for surface expressions of vascular markers (e.g., CD31, CD146, CD144, CXCR4, CD90, and CD105); which demonstrated no significant differences.

FIGS. 13A-13C are a series of images and plots demonstrating in vitro vascular function of primed DVP vs. N-DVP. FIG. 13A: Representative staining images of β-galactosidase senescent cell assay at passage 4 post re-plating of primed VP vs. N-VP from a normal fibroblast-hiPSC (C1.2), and primed DVP vs N-DVP from a diabetic hiPSC line (E1C1). FIG. 13B: Representative flow cytometry analysis of EdU assays at passage 1 and passage 3 post re-plating of primed DVP vs. N-DVP from a diabetic hiPSC line (E1C1) (see FIG. 4E). FIG. 13C: Matrigel vascular tube formation assay demonstrating that N-DVP formed longer and more mature types of tubes than primed DVP.

FIG. 14 is a Western blot analysis of DNA damage response (DDR) proteins following treatment of primed VP and N-VP with NCS. Purified and re-plated DVP vs. N-DVP differentiated from isogenic primed DhiPSC vs. N-DhiPSC (line E1C1) or VP vs N-VP differentiated from isogenic primed vs naïve normal fibroblast-hiPSC (C2); with (+) and without (−) NCS treatment prior cell lysate collection. P-DNA-PK: phosphorylated DNA-PK; P-H2AX: phosphorylated H2AX; NCS: neocarzinostatin.

FIGS. 15A-15D is a series of images demonstrating human VP cell therapy of murine ischemic retinopathy following ischemia-reperfusion (I/R) injury. Representative high magnification images from whole mount retinae demonstrating more abundant survival of HNA⁺ N-DVP cells in the superficial vascular layers of the retina relative to primed DVP at (FIG. 15A) 7 days and (FIG. 15B) three weeks following parallel intra-vitreal injections of 50,000 DVP or N-DVP cells per eye. FIG. 15B: Retinal vascular regions at 3 weeks demonstrated that HNA⁺ human DVP cells were located either abundantly in suspension within vitreous or had migrated and engrafted into vascular abluminal regions of murine ischemic vessels (arrow heads). Scale bars=50 μm. Whole mount retinae were also stained with anti-murine CD31 (mCD31) or anti-murine collagen IV (mCoIV) antibodies for z-stack and maximum-intensity projection imaging. (FIGS. 15C, 15D) Human vascular engraftment of murine retinae, at 2 weeks post DVP injection. (FIG. 15C: Whole mount retinal staining of mouse CD31 (mCD31) and co-staining with murine collagen type-IV (mCol-IV) at 2 weeks following I/R injury and naïve (N-DVP) injections; (FIG. 15D) co-staining with human CD34 (hCD34)-specific and murine collagen type-IV (mCol-IV) antibodies of similar whole mount retinal samples demonstrated that a subset of these mCD31⁺mCol⁺ murine vessels had also formed CD34⁺ human-murine vascular chimerism.

FIGS. 16A, 16B are a series of images showing a method of quantitating human DVP cell engraftment in the vasculature of the neural layers of ischemia-injured mouse retinae. FIG. 16A: Tile-scanned image of cryo-sectioned retina. Multiple images were taken in 20× magnification from each cryo-sections as well as spaced (serial sectioned) retina sections for quantification studies. Hatched areas demonstrate representative example of neural retinal regions evaluated for human vascularization of murine blood vessels. FIG. 16B: Method of regional separation of neural retinal layers employed for quantification of human CD34⁺ cells (hCD34) for data presented in FIGS. 7B and 7D via automated counts using Fiji distribution of ImageJ software.

FIGS. 17A-17D. RNA-Seq transcriptional profiling of primed vs naïve VP from normal and diabetic donors. (FIG. 17A) PCA of whole genome transcriptomes from RNA-Seq samples from primed VP/DVP vs N-VP/DVP, and their parental isogenic hPSC lines (i.e., primed or naive hESC-derived VP, primed or naïve hiPSC-derived VP, and primed or naïve DhiPSC-derived DVP). (FIG. 17B) Heatmap-cluster dendrogram of the top 500 most differentially expressed genes. Shown is hierarchical clustering (Euclidian distance) of isogenic primed vs naïve VP RNA-Seq samples. VP samples are same is in PCA (n=8 VP/DVP and n=8 isogenic N-VP/N-DVP). (hESC-derived VP or N-VP: ‘E’; hiPSC-derived VP or N-VP: “I; DhiPSC-derived DVP or N-DVP: ‘D’). (FIG. 17C) Volcano Plot of differentially-expressed transcripts in whole genome of primed vs naïve VP; log₁₀ p-values vs log 2 fold change in expressions. RNA-Seq VP samples are same as in PCA above (n=8). (FIG. 17D) GSEA of pathways enriched in primed VP vs N-VP. VP samples used for analysis are same as PCA (n=8 VP/DVP and n=8 isogenic N-VP/N-DVP).

FIGS. 18A-18D are a series of plots and graphs demonstrating the lineage-primed gene expression and epigenetic configurations of PRC2-regulated bivalent promoters in primed vs naïve DhiPSC. FIG. 18A: Crossplot of mean genome-wide gene expression of PRC2 module gene targets (Table 1) vs. CpG methylation of non-diabetic N-hiPSC vs. their isogenic primed isogenic hiPSC counterparts (n=6 normal hiPSC lines; Table 1). Plotted are the differentially methylated region (DMR) CpG methylation beta values of PRC2 module promoter regions in LIF-3-reverted hiPSC minus their isogenic primed hPSC counterparts (y-axis, p<0.05) vs. their corresponding differential gene expressions for the same genes (red, x-axis, log 2 fold changes (FC); p≤0.05, FC+/−≥1.5.). FIG. 18B: Curated GSEA pathways for gene targets of the PRC2 module over-represented (FDR<0.01; p<0.001) in LIF-3i-reverted fibroblast-hiPSC vs. their isogenic primed counterparts (n=4; normal hiPSC lines—Table 1). FIG. 18C: Comparison of differentially expressed (p≤0.05, fold change (FC)+/−≥1.5) lineage-primed transcriptional targets of the PRC2 module (Table 1) in naïve vs primed hiPSC (n=6 independent lines). FC: normalized ratios of naïve/primed expression microarray signal intensities show broadly decreased expressions of PRC2 targets in N-hiPSC lines. Ratios are of LIF-3i-reverted vs. primed hPSC samples. FIG. 18D: CHiP-qPCR for H3K27me3 and H3K4me3 histone marks at key multi-lineage developmental promoters in primed vs N-DhiPSC line E1C1. Data is presented as percent input materials between naïve and primed D-hiPSC line E1C1. Standard error of the mean (SEM) bar represents replicates. ChiP-PCR primer sequences and sources are detailed in Table 1.

### DETAILED DESCRIPTION

The disclosure relates to the production of human naïve pluripotent stem cells and their uses in regenerative medicine. In particular, the disclosure relates to reprogramming of patient donor cells to a tankyrase inhibitor-regulated naïve human pluripotency in regenerative medicine. The naïve human pluripotent stem cells when implanted into a subject result in the erasing of dysfunctional epigenetic memory sustained from chronic diseased states, such as, for example, diabetes.

Human Pluripotent Stem Cells

Pluripotent stem cells are classified into naïve and primed based on their growth characteristics in vitro and their potential to give rise to all somatic lineages and the germ line in chimeras. Both are states of pluripotency, but exhibit slightly different properties. The naïve state represents the cellular state of the preimplantation mouse blastocyst inner cell mass, while the primed state is representative of the post-implantation epiblast cells. These two cell types exhibit clearly distinct developmental potential, as evidenced by the fact that naïve cells are able to contribute to blastocyst chimeras, while primed cells cannot. However, the epigenetic differences that underlie the distinct developmental potential of these cell types remain unclear.

One critical variable impacting the differentiation efficiency and functional pluripotency of conventional hiPSC is the developmental, biochemical, and epigenetic commonality of hiPSC with ‘primed’ murine post-implantation epiblast stem cells (mEpiSC), which possess a more restricted pluripotency than inner cell mass-derived mouse ESC (mESC). Conventional hiPSC cultures adopt a spectrum of mEpiSC-like pluripotent states with highly variable lineage-primed gene expressions and post-implantation primed epiblast epigenetic marks that result in inconsistent or diminished differentiation^{12,13} Moreover, epigenetic aberrations in diseased states such as diabetes further inhibit efficient donor cell reprogramming to functional pluripotent states¹⁴⁻¹⁸.

Naïve hiPSC (N-hiPSC) with more primitive pre-implantation epiblast phenotypes, decreased lineage priming, improved epigenetic stability, and higher functionality of differentiated progenitors may solve these obstacles, but this potential has not yet been demonstrated. Several groups have reported various complex small molecule approaches that putatively captured human ‘naïve-like’ pluripotent molecular states that are more primitive than those exhibited by conventional, primed hiPSC (reviewed in ¹¹). However, many of these human naïve-like states exhibited karyotypic instability, global loss of parental genomic imprinting, and impaired multi-lineage differentiation performance.

In the examples section which follows, it was demonstrated for the first time that the epigenetic obstacle of lineage priming and high interline variability of vascular lineage differentiation from normal and diseased conventional hiPSC can be eliminated by reversion to a tankyrase/PARP inhibitor-regulated naïve pluripotent state. Notably, naïve diabetic VP (N-DVP) differentiated from patient-specific naïve diabetic hiPSC (N-DhiPSC) maintained greater genomic stability, higher expression of vascular identity markers, decreased non-lineage gene expression, and were superior in migrating to and re-vascularizing the deep neural layers of the ischemic retina than conventional diabetic VP generated from the same genotype-identical (isogenic) DhiPSC lines. Naïve VP (N-VP) will have great potential for treatment of vascular ischemic disorders and reprogramming of patient donor cells to a tankyrase inhibitor-regulated naïve human pluripotency will a have wide impact in regenerative medicine by more effectively erasing dysfunctional epigenetic memory sustained from chronic diseased states such as diabetes.

Accordingly, in certain embodiments a method of producing a vascular progenitor (VP) cell comprises contacting a human induced pluripotent stem cell (hiPSC) with a composition comprising a leukemia inhibitory factor (LIF) and at least one agent or a combination of at least three agents which inhibit one or more signaling pathways to produce a naïve human induced pluripotent stem cell (N-hiPSC); and, differentiating the N-hiPSC in vitro or by implantation in vivo. In certain embodiments, the hiPSCs are derived from fibroblasts, cord blood cells, human adult or fetal stem cells, bone marrow cells, human induced pluripotent stem cell lines or combinations thereof. In certain embodiments, the hiPSC are derived from diabetic donor hiPSCs (DhiPSC) or non-diabetic donor hiPSCs. In certain embodiments, the hiPSCs are autologous, isogenic, allogeneic, haplotype matched, haplotype mismatched, haplo-identical, xenogeneic or cell lines.

The pluripotent stem cells are not particularly limited, and examples thereof include embryonic stem cells (ES cells), nuclear-transfer embryonic stem (ntES) cells derived from cloned embryo, germline stem cells (GS cell), epiblast cells, embryonic germ cells (EG cells), multipotent germline stem cells (mGS cells), and induced pluripotent stem cells (iPS cells). In certain embodiments, the iPS cells are primed hiPSCs.

The pluripotent stem cells can be derived from any source including for example, human, non-human animals (for example, monkey, sheep, cow, horse, dog, cat, rabbit, rat, and mouse).

Human iPSCs may be derived from different cell types. For example, the cells can be produced from Fibroblast's, keratinocytes, adipose cells, bone marrow stromal cells or neuronal cells, particularly neuronal stem cells. Human iPSCs may be derived from diploid cells which may be a ‘wild-type’ or non-transformed cell. In other embodiments an iPSC is derived from a transformed (tumor) cell.

In certain embodiments, the cell to be reprogrammed is a human cell, e.g. donor-derived. The human cells may be a pluripotent stem cell (hPSC). hPSCs may be induced (iPSCs) or embryo-derived. Cells may be tissue biopsy samples that are initially reprogrammed by standard methods e.g. via a non-integrating vector system such as Sendai virus, then reset using the methods described herein.

Cells may be obtained from pre-existing cell lines without need for biopsy. For example the invention is applicable to pre-existing embryonic stem cell lines. Human embryo-derived stem cells are obtainable from established cell lines such as the Shef6 embryonic cell lines.

In some embodiments cells are derived directly from embryos. Cells derived directly from embryos may be reset using the methods described herein. In some embodiments cells derived directly from embryos are propagated/sustained using the methods embodied herein.

In some embodiments cells are derived from pre-implantation stages. The embryos may be developed in vitro or in utero are cultured either intact, or after isolation of the inner cell mass (ICM) by microdissection or immunosurgery. Optionally the cells are dissociated into single cells prior to use in the methods of the present disclosure.

In some embodiments cells are derived from late blastocysts, peri-implantation embryos or post-implantation epiblast. Epiblast cells may be dissected and/or dissociated prior to use in the methods of the invention.

Human embryonic stem cells may be obtained using methods which do not require destruction of the embryo. For example, embryonic stem cells may be obtained from the human embryo by biopsy. Methods for obtaining embryonic stem cells from the embryo without destruction of the embryo were disclosed for example in Klimanskaya I. et al., 2006. Nature 444, 481-485.

In some embodiments of the present invention, the methods and uses do not involve destruction of human embryos. In some embodiments, the methods do not involve or use cells obtained by methods requiring destruction of human embryos.

Cells may be obtained from an individual by standard techniques, for example by biopsy for skin cells. Cells may preferably be obtained from an adult. Methods for generating iPSCs are known in the art, for example as described in: Takahashi et al Nature 2007; Yu et al, Science 2007.

The cell to be reprogrammed may also be a cell which already expresses one of the reprogramming factors.

It will be understood that the methods and uses described herein also apply to other primates and non-human mammalian cells, and the features of the methods, uses and reset cells as described herein apply to non-human mammalian cells mutatis mutandis. Put another way, it will be understood (unless context demands otherwise) that where the term “human” is recited herein, it can be replaced with “mammalian” or any of the following: primate; non-human mammalian non-human primate; pig; sheep; cat; dog; goat; cow; camel; horse; llama; alpaca etc.

Reversion of Primed hiPSCs: In some embodiments, the reversion of conventional, primed normal non-diabetic hiPSC or DhiPSC results in changes from flattened) to dome-shaped SSEA4⁺TRA-1-81⁺ N-DhiPSC colony morphologies. In embodiments, the primed to naïve transition is accompanied by activation of protein expressions of a panoply of naïve-specific pluripotency factors comprising NANOG, KLF2, NR5A2, TFCP2L1, STELLA/DPPA3, E-CADHERIN and the like, as well as naïve ESC-specific proteins, that included phosphorylated STAT3 and TFAP2C.

In some embodiments, the induction of a naïve state further comprises expressing reprogramming factors in the cell (e.g. KLF2 and NANOG). In preferred embodiments the reprogramming factors are human factors. In some embodiments, other reprogramming factors may be used. For example, other factors which are known to play a role in programming of pluripotent stem cells may be used. Accordingly, in some embodiments, reprogramming factors for use in the present invention include one or more of OCT3/4, SOX2, Klf4, LIN28, c-MYC, KLF2 and NANOG. In some embodiments, reprogramming factors for use in the present invention include one or more of KLF4, NR5a1 KLF17, NANOG and KLF2. In some embodiments, reprogramming factors for use in the present invention include one or more of KLF4, NR5a1 KLF17, NANOG, OCT3/4, SOX2, LIN28, c-MYC and KLF2.

Expression of the reprogramming factors is suitably achieved using genetic material introduced into the cells and containing coding sequences for the reprogramming factors operatively linked to promoters; preferably, plasmids are used. The promoters direct expression of the reprogramming factors and, generally, a constitutive promoter is suitable, but the choice of promoter is not critical provided the reprogramming factors are expressed in the cells. Examples of suitable promoters include CAG, PKG and CMVE. The genetic material, such as the plasmids, further preferably does not replicate and has a very low integration efficiently, which can be further reduced e.g. by using circular rather than linear plasmids.

Plasmids can be introduced by using nucleofection which is an established procedure and known to be efficient. Other chemical and electrical methods are known and are also efficient, including electroporation and lipofection. Different transfection methods and protocols are available for different cells, all well known in the art. Generally, it is believed that the choice of plasmid and promoter and transfection route is not critical to the invention. The plasmid preparation comprises one or more plasmids which express in the cell the one or more reprogramming factors. There may be one plasmid for each factor or a plasmid may express more than one or all factors.

Once the N-hiPSCs have been obtained, the N-hiPSCs are maintained in a naïve state for use in transplantation in a subject in need thereof. Accordingly, in one embodiment, a method of sustaining/maintaining/propagating human stem cells in a naïve state, comprises inhibiting PKC and MAPK/ERK/MEK. In certain embodiments, the method further comprises treatment with a STAT3 activator. In a certain embodiments, the STAT3 activator is LIF, for example human LIF.

Inhibitors: In certain embodiments, the composition comprises a leukemia inhibitory factor (LIF) and a combination of at least three agents comprising inhibitors of poly-ADP-ribosyltransferase, MEK, GSK3 and signaling pathways thereof. In certain embodiments, the poly-ADP-ribosyltransferase is tankyrase. In certain embodiments, the GSK3 is a GSK30 isoform. In certain embodiments, a tankyrase inhibitor comprises: XAV939, IWR-1, G007-LK, JW55, AZ1366, JW 74, NVP-TNKS656 or combinations thereof. In certain embodiments, a GSK3β inhibitor comprises: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR 99021), 5-Ethyl-7,8-dimethoxy-1H-pyrrolo[3,4-c]isoquinoline-1,3(2H)-dione (3F8), 1-(7-Methoxyquinolin-4-yl)-3-[6-(trifluoromethyl)pyridin-2-yl]urea (A 1070722), N6-[2-[[4-(2,4-Dichlorophenyl)-5-(1H-imidazol-1-yl)-2-pyrimidinyl]amino]ethyl]-3-nitro-2,6-pyridinediamine (CHIR 98014), lithium chloride (LiCl), 4-benzyl-2-methyl-1,2,4-thiadiazolidine-3,5-dione (TDZD-8), 5-iodo-indirubin-3′-monoxime (I3′M) and N-(4-methoxybenzyl)-N′-(5-nitro-1,3-thiazol-2-yl)urea (AR-A014418) or combinations thereof. In certain embodiments, an MEK inhibitor comprises: PD032590, CI-1040 (PD184352), cobimetinib (GDC-0973, XL518), Selumetinib (AZD6244), MEK162, AZD8330, TAK-733, GDC-0623, Refametinib (RDEA119; BAY 869766), Pimasertib (AS703026), RO4987655 (CH4987655), RO5126766, WX-554, HL-085 or combinations thereof.

It will be understood that other kinase inhibitors which inhibit a kinase responsible for an intracellular signaling component of the same cascades (e.g. MAPK/ERK, for example ERK1 or ERK2 cascade) may be substituted where desired for the MEK inhibitor or GSK3 inhibitor. This may include inhibition of an upstream stimulus of the MAPK pathway, in particular through the FGF receptor (Ying Q. L. et al., Nature. 2008 May 22; 453(7194):519-23). Likewise the LIF may be substituted where desired for other activators of STAT3 or gp130 signaling.

Inhibitors may be provided or obtained by those skilled in the art by conventional means or from conventional sources, and such inhibitors per se are not part of the present invention (see also WO2007113505).

Reference to GSK3 inhibition refers to inhibition of one or more GSK3 enzymes. The family of GSK3 enzymes is well-known and a number of variants have been described (see e.g. Schaffer et al.; Gene 2003; 302(1-2): 73-81). In specific embodiments GSK3-β is inhibited. GSK3-α inhibitors are also suitable, and in general inhibitors for use in the invention inhibit both GSK3-α and GSK3-β. A wide range of GSK3 inhibitors are known, by way of example, the inhibitors CHIR 98014, CHIR 99021, AR-AO144-18, TDZD-8, SB216763 and SB415286. In addition, the structure of the active site of GSK3-β has been characterized and key residues that interact with specific and non-specific inhibitors have been identified (Bertrand et al., J Mol Biol. 2003; 333(2): 393-407). This structural characterization allows additional GSK inhibitors to be readily identified.

In certain embodiments, the inhibitors used herein are specific for the kinase to be targeted. The inhibitors of certain embodiments are specific for GSK3-β and GSK3-α, substantially do not inhibit erk2 and substantially do not inhibit cdc2. In certain embodiments, the inhibitors have at least 100 fold, at least 200 fold, at least 400 fold selectivity for human GSK3 over mouse erk2 and/or human cdc2, measured as ratio of IC₅₀ values; here, reference to GSK3 IC₅₀ values refers to the mean values for human GSK3-β or GSK3-α. In some embodiments, the GSK3β inhibitor is CHIR 99021 which is specific for GSK3. Examples of GSK3 inhibitors are described in Bennett C, et al., J. Biol. Chem., vol. 277, no. 34, Aug. 23, 2002, pp 30998-31004 and in Ring D B, et al., Diabetes, vol. 52, March 2003, pp 588-595. Suitable concentrations for use of CHIR 99021 are in the range of about 0.01 to about 10, about 0.1 to about 5, about 0.1 to about 11 μM.

Reference to a MEK inhibitor herein refers to MEK inhibitors in general. Thus, reference to a MEK inhibitor refers to any inhibitor a member of the MEK family of protein kinases, including MEK1, MEK2 and MEK5. Reference is also made to MEK1, MEK2 and MEK5 inhibitors. Examples of suitable MEK inhibitors, already known in the art, include the MEK1 inhibitors PD184352 and PD98059, inhibitors of MEK1 and MEK2 U0126 and SL327, and those discussed in Davies et al. (2000) (Davies S P, et al. Biochem J. 351, 95-105). In particular, PD184352 and PD0325901 have been found to have a high degree of specificity and potency when compared to other known MEK inhibitors (Bain J et al., Biochem J. 2007 Dec. 15; 408(3):297-315). Other MEK inhibitors and classes of MEK inhibitors are described in Zhang et al. (2000) Bioorganic & Medicinal Chemistry Letters; 10:2825-2828.

Tankyrase inhibitors suitable for use in the present disclosure comprises: XAV939, IWR-1, G007-LK, JW55, AZ1366, JW 74, NVP-TNKS656 or combinations thereof. Tankyrases are involved in a number of cellular functions, that includes telomere homeostasis, mitotic spindle formation, vesicle transport linked to glucose metabolism, Wnt/β-catenin signaling, and viral replication.

In some embodiments, the tankyrase inhibitor is XAV939.

In certain embodiments, a composition comprises LIF and one or more inhibitors of tankyrase, mitogen-activated protein kinase kinase (MEK), Glycogen Synthase Kinase 3-β (GSK3β) or signaling pathways thereof. In certain embodiments a tankyrase inhibitor comprises: XAV939, IWR-1, G007-LK, JW55, AZ1366, JW 74, NVP-TNKS656 or combinations thereof. In certain embodiments, a GSK3β inhibitor comprises: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR 99021), 5-Ethyl-7,8-dimethoxy-1H-pyrrolo[3,4-c]isoquinoline-1,3(2H)-dione (3F8), 1-(7-Methoxyquinolin-4-yl)-3-[6-(trifluoromethyl)pyridin-2-yl]urea (A 1070722), N6-[2-[[4-(2,4-Dichlorophenyl)-5-(1H-imidazol-1-yl)-2-pyrimidinyl]amino]ethyl]-3-nitro-2,6-pyridinediamine (CHIR 98014), lithium chloride (LiCl), 4-benzyl-2-methyl-1,2,4-thiadiazolidine-3,5-dione (TDZD-8), 5-iodo-indirubin-3′-monoxime (I3′M) and N-(4-methoxybenzyl)-N′-(5-nitro-1,3-thiazol-2-yl)urea (AR-A014418) or combinations thereof. In certain embodiments, an MEK inhibitor comprises: PD032590, CI-1040 (PD184352), cobimetinib (GDC-0973, XL518), Selumetinib (AZD6244), MEK162, AZD8330, TAK-733, GDC-0623, Refametinib (RDEA119; BAY 869766), Pimasertib (AS703026), RO4987655 (CH4987655), RO5126766, WX-554, HL-085 or combinations thereof.

In some embodiments, other inhibitors may be included, for example a MEK inhibitor, a GSK3 inhibitor, a STAT3 activator, an FGF inhibitor (e.g. PD173074), a HDAC inhibitor, a tankyrase inhibitor, a ROCK inhibitor (e.g. Y27632), a PKC inhibitor or combinations thereof. In certain embodiments, the STAT3 (signal transducer and activator of transcription 3) activator is LIF, e.g. human LIF (hLIF).

PKC inhibitors suitable for use in the present invention include 3-[1-[3-(dimethylamino)propyl]-5-methoxy-1H-indol-3-yl]-4-(1H-indol-3-yl)-1H-pyrrole-2,5-dione (Go6983), Ro-31-8425.

Inhibition of MEKs, GSK3 and tankyrase can also be conveniently achieved using RNA-mediated interference (RNAi). Typically, a double-stranded RNA molecule complementary to all or part of a MEK gene, GSK3β or tankyrase is introduced into the stem cells, thus promoting specific degradation of MEK-encoding mRNA molecules, GSK3β-encoding mRNA molecules or tankyrase-encoding mRNA molecules. This post-transcriptional mechanism results in reduced or abolished expression of the targeted MEK gene. Suitable techniques and protocols for achieving inhibition using RNAi are known.

Accordingly, references herein to an inhibitor herein, encompass RNAi as an inhibitor.

A number of assays for identifying kinase inhibitors, including GSK3 inhibitors and MEK inhibitors, are known. For example, Davies et al (2000) describe kinase assays in which a kinase is incubated in the presence of a peptide substrate and radiolabelled ATP. Phosphorylation of the substrate by the kinase results in incorporation of the label into the substrate. Aliquots of each reaction are immobilized on phosphocellulose paper and washed in phosphoric acid to remove free ATP. The activity of the substrate following incubation is then measured and provides an indication of kinase activity. The relative kinase activity in the presence and absence of candidate kinase inhibitors can be readily determined using such an assay. Downey et al. (1996) J Biol Chem. 271(35): 21005-21011 also describes assays for kinase activity which can be used to identify kinase inhibitors.

Methods of Treatment and Uses Thereof

The naïve human induced pluripotent stem cells described herein are isogenic, homogeneity and absence of lineage priming or epigenetic restrictions that bias differentiation behavior.

The N-hiPSCs can be implanted into a subject in treatments wherein regeneration of tissues is of benefit to the subject. For example, vascular tissues, ischemic retinal tissues, damaged nerve tissue, etc., can be normalized by transplanting the N-hiPSCs which differentiate into mature cells and repopulate the damaged tissue. Examples of diseases which would benefit from the regenerative potential of the N-hiPSCs include Parkinson's disease, Alzheimer's disease, retinal pigmentary degeneration, amyotrophic lateral sclerosis, optic neuromyelitis, optic neuritis, acute disseminated encephalomyelitis, allergic encephalomyelitis, spinal cord damage, transverse myelitis, spinocerebellar degeneration, chronic inflammatory demyelinating encephalopathy (CIDP), Guillain-Barre syndrome, multiple sclerosis, epilepsy, Parkinson's syndrome, Down syndrome, schizophrenic disorder, neurodystonia, Huntington's disease, diabetic retinopathy, age-related macular degeneration, and inner ear deafness.

In other embodiments, the naïve pluripotent stem cells can be used for screening drug candidate compounds for various diseases. For example, by adding the drug candidate compounds singly or in combination with other drugs into the differentiation-induced cells, the morphology or functional change of the cells, increase and decrease of various factors, gene expression profiling, and the like, are detected so as to carry out evaluation. Herein, the N-hiPSCs are cells having the same phenotype as that of disease to be treated, and differentiation-induced from the naive pluripotent stem cells produced from cells derived from a patient having a disease.

Further uses include: differentiation to create cell culture models of human development and disease that can be applied in drug discovery and development, and in teratogenicity and toxicology testing; source of tissue stem cells and more mature cells for applications in clinical cell therapy; analysis of the relative contributions of genetics and epigenetics to developmental disorders, genetic disease and quantitative traits to facilitate advances in diagnostics, prognostics and patient treatment; generation of tissues and organs for transplantation either by bioengineering in vitro or by lineage/organ specific contribution to human-animal chimaeras.

Naïve induced pluripotent stem cells can also be derived from rom non-human primates and other mammals for use in precision genome engineering to enhance or modify germline genetic constitution of animals. Germline modification is achieved by genome engineering or genome editing and clonal selection of ground state cells in culture, followed by production of chimaeras, breeding and screening for transmission of the modified genotype. Desired genetic alterations include single or multiple gene deletion, point mutation, or substitution. Chromosome-scale genome modifications/substitutions are also possible. Applications include: disease models; behavioral models; host compatibility for xenotransplantation and organ substitution; pharmaceutical, antibody and vaccine production; livestock improvement; breeding stock preservation and improvement. Non-human primate ground state cells may also be used in pre-clinical testing and evaluation of cell therapies.

The following examples are offered by way of illustration and not by way of limitation.

### EXAMPLES

### Example 1: Vascular Progenitors Generated from Tankyrase Inhibitor-Regulated Naïve Diabetic Human iPSC Potentiate Efficient Revascularization of Ischemic Retina

The studies herein, describe for the first time the advantage of employing an alternative tankyrase/PARP inhibitor-regulated human naïve pluripotent state for improving vascular regenerative therapies. Tankyrase/PARP inhibitor-regulated N-hiPSC represent a new class of human stem cells for regenerative medicine with improved multi-lineage functionality. In contrast, conventional hiPSC cultures adopt transcriptomic, epigenetic, and signaling signatures of lineage-primed pluripotency, and display a heterogeneous propensity for lineage bias and differentiation.

Materials and Methods

Bioethics. hESC lines used in these studies as controls for hiPSC were obtained commercially from the Wisconsin International Stem Cell Bank (WISCB). All hESC experiments proposed conform to guidelines outlined by the National Academy of Sciences, and the International Society of Stem Cell Research (ISSCR). Commercially-acquired hESC are under purview of the Johns Hopkins University (JHU) Institutional Stem Cell Research Oversight (ISCRO), and conform to Institutional standards regarding informed consent and provenance evaluation. All experiments proposed received approval by the JHU ISCRO committee. All animal use and surgical procedures were performed in accordance with protocols approved by the Johns Hopkins School of Medicine Institute of Animal Care and Use Committee (IACUC) and the Association for Research of Vision and Ophthalmology statement for the Use of Animals in Ophthalmic and Visual Research.

Conventional primed (E8) and naïve (LIF-3i) cultures of hESC and hiPSC. All human embryonic stem cells (hESC) and hiPSC lines used in these studies were maintained and expanded in undifferentiated conventional feeder-free primed states in Essential 8 (E8) medium, or naive-reverted with the LIF-3i system, as described^{12,13}.

Conventional cultures of hiPSC were propagated using commercial E8 medium (ThermoFisher Scientific), or an in-house variant formulation consisting of DMEM/F-12 supplemented with 2.5 mM L-Glutamine, 15 mM HEPES and 14 mM sodium bicarbonate (ThermoFisher Scientific, cat #11330), 50-100 ng/mL recombinant human FGF-basic (Peprotech), 2 ng/mL recombinant human TGF-β1 (Peprotech), 64 μg/mL L-ascorbic acid-2-phosphate magnesium (Sigma), 14 ng/mL sodium selenite (Sigma), 10.7 μg/mL recombinant human transferrin (Sigma), and 20 μg/mL recombinant human insulin (Peprotech). Conventional hiPSC were expanded in E8 onto Vitronectin XF (STEMCELL Technologies) matrix-coated tissue culture-treated 6-well plates (Corning). E8 medium was replaced daily and hiPSC were gently passaged every 5-6 days by mechanical selection or bulk passaged using non-enzymatic reagents (i.e., Versene solution (ThermoFisher Scientific) or Phosphate-Buffer-Saline (PBS)-based enzyme-free cell dissociation buffer (ThermoFisher Scientific, #13151).

LIF-3i medium was prepared fresh every other week and consists of DMEM/F-12 supplemented with 20% KnockOut Serum Replacement (KOSR, ThermoFisher Scientific), 0.1 mM MEM non-essential amino acids (MEM NEAA, ThermoFisher Scientific), 1 mM L-Glutamine (ThermoFisher Scientific), 0.1 mM β-mercaptoethanol (Sigma), 20 ng/mL recombinant human LIF (Peprotech), 3 μM CHIR99021 (Tocris or Peprotech), 1 μM PD0325901 (Sigma or Peprotech), and 4 μM XAV939 (Sigma or Peprotech). Prior to switching between E8 and LIF-3i media, hPSC were adapted for one passage in LIF-5i, as described^{12,13} LIF-5i was prepared by supplementing LIF-3i with 10 μM Forskolin (Stemgent or Peprotech), 2 μM purmorphamine (Stemgent or Peprotech) and 10 ng/mL recombinant human FGF-basic (Peprotech). Briefly, primed hiPSC were adapted overnight by substituting E8 with LIF-5i medium. The next day, hiPSC were enzymatically dissociated (Accutase, ThermoFisher Scientific) and transferred onto irradiated mouse embryonic fibroblast (MEF) feeders in LIF-5i medium for only one passage (2 to 3 days). All subsequent passages were grown in LIF-3i medium on MEF feeders. Isogenic E8 cultures were maintained in parallel for simultaneous phenotypic characterization, as previously described in detail^{12,13}.

Reprogramming of diabetic fibroblasts to conventional DhiPSC. Adult human Type-I diabetic (T1D) fibroblasts obtained with patients' informed consent, were purchased from DV Biologics, and cultured in fibroblast culture medium (I-Gro medium, DV Biologics). For reprogramming, single cells were obtained using Accutase and counted. Episomal expression of seven genes (SOX2, OCT4, KLF4, c-MYC, NANOG, LIN28, SV40LT) was accomplished by nucleofection of 1×10⁶ diabetic fibroblast cells with 2 μg each of three plasmids, pCEP4-EO2S-EN2L, pCEP4-EO2S-ET2K, and pCEP4-EO2S-EM2K as described^{27,28}. Fibroblasts were nucleofected using human dermal fibroblast nucleofector kits (Lonza, VPD-1001) with Amaxa nucleofector program U-023. Nucleofected cells were transferred onto irradiated MEF in fibroblast growth medium supplemented with 10 μM Rho-associated, coiled-coil containing protein kinase (ROCK) inhibitor Y27362 (Stemgent). The next day, 2 mL of DMEM/F-12 supplemented with 20% KOSR, 0.1 mM MEM NEAA, 1 mM L-Glutamine, 0.1 mM β-mercaptoethanol, 50 ng/mL bFGF, 10 μM Y27362, 5 μg/mL ascorbic acid, and 3 μM CHIR99021 was added. Half of the medium was replaced with fresh medium without Y27362 every other day, until hiPSC colonies appeared. Individual hiPSC colonies were manually isolated, further expanded onto vitronectin-coated plates in E8 medium, or cryopreserved.

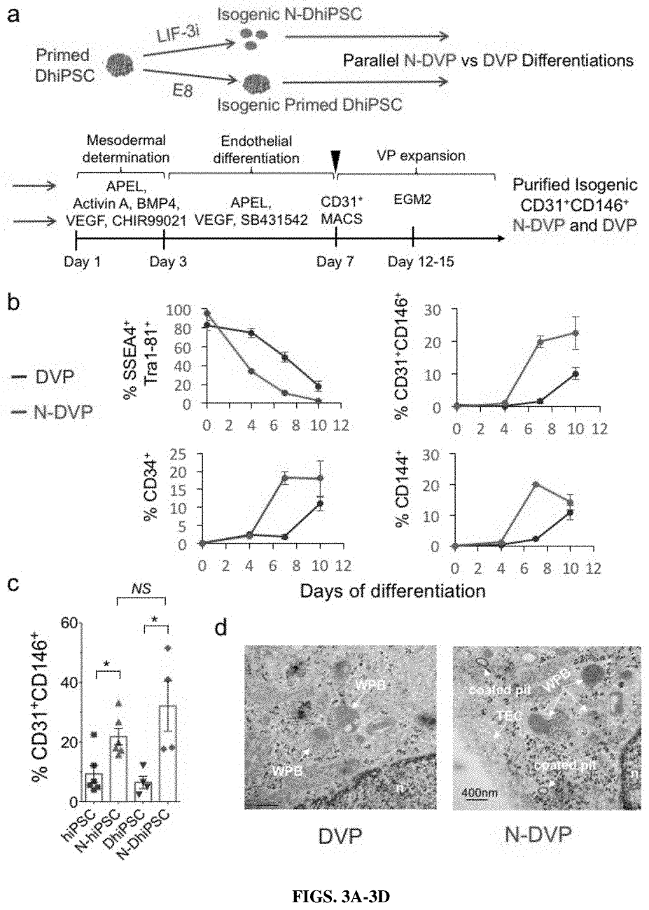

Parallel isogenic primed hiPSC vs. N-hiPSC directed neuroectodermal, endodermal, and vascular differentiations in vitro. To examine the differentiation competence of normal and diabetic N-hiPSC, we directly differentiated LIF-3i-reverted naïve vs their primed genotypically-identical isogenic (same line) sibling hiPSC counterparts in parallel, as previously described without additional cell culture manipulations^{12,13} “Re-priming” (i.e., converting N-hiPSC back to conventional primed conditions prior to their use in directed differentiation assays^{25,26}) was not necessary with the LIF-3i method^{12,13}. To minimize hiPSC assay variations within directed differentiation experiments that may arise from hiPSC interline variability and genetic background bias, paired isogenic primed and LIF-3i-reverted hiPSC lines were simultaneously and directly cultured into defined, identical, feeder-free differentiation systems according to manufacturer's directions. Naïve reversions were performed in LIF-5i/LIF-3i media fresh for each differentiation experiment starting from a low passage primed hPSC line, as described¹³. For example, for functional comparisons of naïve vs. primed isogenic hiPSC lines, sibling cultures were prepared at equivalent passage number, starting from the primed parental hPSC line. Primed and naïve hPSC sibling cultures were expanded in parallel in their respective media for 5-7 passages before differentiation (e.g., E8 vs. LIF-3i, see schematic FIG. 3A). This experimental approach for primed vs naïve differentiation was previously employed for functional comparison of primed vs naïve hiPSC states^{12,13} Detailed information for the origins and derivation of all hiPSC lines used in these studies for these assays was previously reported¹².

Differentiation to neural progenitors was performed using GIBCO PSC neural induction medium (NIM; ThermoFisher Scientific, A1647801) and the manufacturer's recommendations. Differentiation into definitive endodermal progenitors was achieved using the StemDiff Definitive Endoderm Kit (StemCell Technologies) following manufacturer's protocols. Vascular differentiation of VP from primed and naïve hiPSC was modified and optimized from methods we previously described 7. The experimental approach is summarized in FIG. 3A. Briefly, VP differentiation was performed using a modified protocol based on the STEMdiff APEL-Li medium system³². Briefly, APEL-2Li medium (StemCell Technologies, #5271) was supplemented with Activin A (25 ng/mL), VEGF (50 ng/mL), BMP4 (30 ng/mL), and CHIR99021 (1.5 μM) for the first 2 days, and then APEL-Li that was supplemented with VEGF (50 ng/mL) and SB431542 (10 μM). Differentiation medium was replaced every 2 days until cells were harvested for analysis.

Isogenic primed vs. naive hiPSC teratoma assays. Isogenic (genotypic-identical; same line) primed (E8) and naïve (LIF-3i) hiPSC cultures were maintained in parallel for 9 passages prior to teratoma formation assays. Teratomas were directly generated from a fixed number of cells (5×10⁶) and duration (8 weeks) in isogenic primed vs. LIF-3i naive hiPSC conditions. LIF-3i-cultured N-hiPSC colonies did not require chemical manipulation or re-priming culture steps prior to enzymatic harvest from culture and direct injection into NOG mice. Adherent primed vs naive hiPSC were collected using Accutase and counted using Countess counter (ThermoFisher Scientific). For all experiments, 5×10⁶ hiPSC were admixed with Growth factor reduced Matrigel (Corning, cat #356230) on ice. Cells were injected subcutaneously into the hind limbs of immunodeficient NOG male sibling mice. Teratomas were dissected 8 weeks following injection and fixed by overnight immersion in PBS, 4% formaldehyde. All tissues were paraffin-embedded, and microsectioned (5 μm thickness) onto microscope glass slides (Cardinal Health) by the Histology laboratory from the Pathology Department at the Johns Hopkins University. To account for heterogeneous teratoma histological distribution, 15 individual equally-spaced sections were immunostained per tissue for each antigen of interest and quantification. Slides were heated in a hybridization oven (ThermoFisher Scientific) at 60° C. for 20 minutes and then kept at room temperature for 1 hour to dry. Paraffin was eliminated by three consecutive immersions in xylenes (Sigma) and sections were rehydrated by transitioning the slides in successive 100%, 95%, 70% and 0% ethanol baths. Sections were placed in 1× wash buffer (Dako) prior to heat-induced antigen retrieval using 1× Tris-EDTA, pH9 target retrieval solution (Dako) and wet autoclave (125° C., 20 min). Slides were cooled and progressively transitioned to PBS. After 2 washes, tissues were blocked for 1 hour at room temperature using PBS, 5% goat serum (Sigma), 0.05% Tween 20. Endogenous biotin receptors and streptavidin binding sites were saturated using the Streptavidin/Biotin Blocking kit (Vector Laboratories). All antibodies were diluted in blocking solution. Sections were incubated overnight at 4° C. with monoclonal mouse anti-NG2 (Sigma, C8035, 1:100), mouse anti-SOX2 (ThermoFisher Scientific, MAS-15734, 1:100) or rabbit anti-cytokeratin 8 (Abcam, ab53280, 1:400) primary antibodies, washed 3 times, incubated for 1 hour at room temperature with biotinylated goat anti-mouse or goat anti-rabbit IgG antibodies (Dako, 1:500), washed 3 times and incubated with streptavidin Cy3 (Sigma, 1:500) for 30 minutes at room temperature. After 2 washes, tissues were incubated for 2 hours at room temperature with a second primary antibody (e.g., anti-Ki67) differing in species from the first primary antibody. After incubation with rabbit (Abcam, ab16667, 1:50) or mouse (Dako, M7240, 1:50) anti-Ki67 monoclonal antibody, sections were washed 3 times and incubated for 1 hour at room temperature with highly cross-adsorbed Alexa Fluor 488-conjugated goat anti-rabbit or goat anti-mouse secondary antibody (ThermoFisher Scientific, 1:250). Sections were washed twice, incubated with 10 μg/mL DAPI (ThermoFisher Scientific, D1306) in PBS, washed 3 times in PBS and slides were mounted with coverslips using Prolong Gold Anti-fade reagent (ThermoFisher Scientific) for imaging. Isotype controls for mouse (ThermoFisher Scientific) and rabbit (Dako) antibodies were substituted at matching concentration with primary antibodies as negative controls.

For teratoma organoid quantifications, photomicrographs were obtained using a 20× objective and Zeiss LSM 510 Meta Confocal Microscope. Teratoma organoid quantifications were first assessed by histological grading of 20 whole cross-sections that were equally spaced throughout the tissue and stained with hematoxylin-eosin. Lineage-specific quantifications were validated in adjacent sections (n=15) by fluorescent immunostains. Image processing and quantification was performed using NIS-Elements software (Nikon). The ROI editor component was applied to autodetect regions of interest in the Cy3 channel that delineated lineage-defined structures (i.e., Cytokeratin 8⁺ definitive endoderm, NG2⁺ chondroblasts, SOX2⁺ neural rosettes) within teratomas. Thresholding and restrictions were standardized in the Object Count component and applied to detect and export the number of DAP⁺ and Ki67⁺ nuclei within ROIs for all analyzed sections.

Antibodies. Source and working dilutions of all antibodies used in these studies for Western blots, FACS, genomic dot blots, ChIP, and immunofluorescence experiments are listed in the following Table 1 (includes Table 1A and 1).

TABLE 1A

| | Antibody species | | Manufacture's | | |
|---|---|---|---|---|---|
| Antibody | specificity | Company | Number | Concentration | Purpose |
| SSEA4-APC | | R&D Systems | FAB1435A | 2 uL/tube (1 × 10⁶ | FACS |
| | | | | cells or less) | |
| Tra 1-81-PE | Mouse anti-human | BD | 560161 | 5 uL/tube (1 × 10⁶ | FACS |
| | | | | cells or less) | |
| human CD31- | Mouse anti-human | eBiosciences | 17-0319-42 | 1 uL/tube (1 × 10⁶ | FACS |
| APC | | | | cells or less) | |
| human CD146- | Mouse anti-human | BD | 550315 | 5 uL/tube (1 × 10⁶ | FACS |
| PE | | | | cells or less) | |
| human CD44- | Mouse anti-human | BD | 550989 | 5 uL/tube (1 × 10⁶ | FACS |
| PE | | | | cells or less) | |
| human CD45- | Mouse anti-human | BD | 555483 | 5 uL/tube (1 × 10⁶ | FACS |
| PE | | | | cells or less) | |
| human Nestin- | Mouse anti-human | BD | 560393 | 5 uL/tube (1 × 10⁶ | FACS |
| A647 | | | | cells or less) | |
| human SOX1- | Mouse anti-human | BD | 561592 | 5 uL/tube (1 × 10⁶ | FACS |
| PE | | | | cells or less) | |
| Cytokeratin 8 | Mouse | Abcam | ab53280 | 1:400 | IF |
| (CK8) | | | | | |
| NG2 | Mouse | Sigma | C8035 | 1:100 | IF |
| human SOX2 | Mouse anti-human | ThermoFisher | MAS-15734 | 1:100 | IF |
| human Ki67 | Mouse anti-human | DAKO | M7240 | 1:50 | IF |
| human Ki67 | Rabbit anti-human | Abcam | ab16667 | 1:50 | IF |
| human Tra1- | Mouse anti-human | Stemgent | 09-0069 | | IF |
| 81-Alexa488 | | | | | |
| NANOG | Rabbit | Cell Signaling | 4903 | | IF |
| NR5A2 | Rabbit | Sigma Aldrich | HPA005455 | 1:100 | IF |
| human | Mouse anti-human | Millipore | MAB4388 | | |
| STELLA | | | | | |
| human E- | Mouse anti-human | DAKO | M3612 | | IF |
| Cadherin | | | | | |
| Human TUJ1- | Mouse anti-human | BD | 560394 | | IF |
| Alexa647 | | | | | |
| Mouse CD31 | Rat anti-mouse | BD | 550274 | | IF |
| Human nuclear | Mouse anti-human | Millipore | MAB1281C3 | 1:100 | IF |
| antigen | | | | | |
| (HNA)-Cy3 | | | | | |
| Human CD34 | Mouse anti-human | BD | 347660 | 1:50 | IF |
| Mouse | Rabbit anti-mouse | Millipore | Ab756P | 1:200 | IF |
| Collagen type | | | | | |
| IV | | | | | |
| DAPI | N/A | | | 1:1000 | IF |
| human CD31 | Mouse anti-human | DAKO | M0823 | 1:50 | IF |
| Phosphorylated | Rabbit | Cell Signaling | 9145 | 1:1000 | Western blot |
| STAT3 | | | | | |
| Total STAT3 | Mouse | Cell Signaling | 9139 | 1:1000 | Western blot |
| TFAP2C | Rabbit | Sigma | SAB2102408 | 1:1000 | Western blot |
| TANK 1/2 | Mouse | Santa Cruz | sc-365897 | 1:1000 | Western blot |
| AXIN-1 | Rabbit | Cell Signaling | 2087 | 1:1000 | Western blot |
| ACTIN | Rabbit | Abcam | Ab6276 | 1:1000 | Western blot |
| Phosphorylated | Rabbit | Cell Signaling | 9718 | 1:200 | IF |
| H2AX | | | | | |
| | | | | 1:1000 | Western blot |
| Total H2AX | Rabbit | Cell Signaling | 7631 | 1:1000 | Western blot |
| RAD51 | Rabbit | Cell Signaling | 8875 | 1:1000 | Western blot |
| RAD 54 | Rabbit | Cell Signaling | 15016 | 1:1000 | Western blot |
| Phosphorylated | Rabbit | Cell Signaling | 2529 | 1:1000 | Western blot |
| P53 | | | | | |
| Total P53 | Mouse | Cell Signaling | 2524 | 1:1000 | Western blot |
| 5mC | Rabbit | Cell Signaling | 28692 | 1:1000 | Dot blot |
| 5hmC | Mouse | Cell Signaling | 51660 | 1:1000 | Dot blot |

TABLE 1B

| Gene | Purpose | | Sequences |
|---|---|---|---|
| ACTB | RT-PCR | Taqman gene expression | Hs99999903_ml |
| | | assay | |
| CD31/PECAMI | RT-PCR | Taqman gene expression | Hs01065282_ml |
| | | assay | |
| CXCR4 | RT-PCR | Taqman gene expression | Hs00607978_sl |
| | | assay | |
| DLLI | RT-PCR | Taqman gene expression | Hs00194509_ml |
| | | assay | |
| Endothelin- | RT-PCR | Taqman gene expression | Hs00174961_ml |
| | | assay | |
| FZD7 | RT-PCR | Taqman gene expression | Hs00275833_sl |
| | | assay | |
| GAPDH | RT-PCR | Taqman gene expression | Hs02758991_gl |
| | | assay | |
| GATA2 | RT-PCR | Taqman gene expression | Hs00231119_ml |
| | | assay | |
| GATA6 | RT-PCR | Taqman gene expression | Hs00232018_ml |
| | | assay | |
| HAND1 | RT-PCR | Taqman gene expression | Hs02330376_sl |
| | | assay | |
| ICAM2 | RT-PCR | Taqman gene expression | Hs00609563_ml |
| | | assay | |
| MSX2 | RT-PCR | Taqman gene expression | Hs00751239_sl |
| | | assay | |
| MYOD1 | RT-PCR | Taqman gene expression | Hs02330075_gl |
| | | assay | |
| NANOG | RT-PCR | Taqman gene expression | Hs02387400_gl |
| | | assay | |
| PAX6 | RT-PCR | Taqman gene expression | Hs01088106_gl |
| | | assay | |
| SOX1 | RT-PCR | Taqman gene expression | Hs01057642_sl |
| | | assay | |
| TIE1 | RT-PCR | Taqman gene expression | Hs00892696_ml |
| | | assay | |
| TIE2/TEK | RT-PCR | Taqman gene expression | Hs00945155_ml |
| | | assay | |
| VWF | RT-PCR | Taqman gene expression | Hs01109446_ml |
| | | assay | |

| | | | SEQ | | SEQ |
|---|---|---|---|---|---|
| | | | ID | | ID |
| Gene | Purpose | Sequences | NO: | | NO: |
| | | Forward | | Reverse | |
| CD31/PECAM1 | ChIP- | TGCAAAGAGCAAAGG | 1 | CACGCCTAGCCAAAATCACT | 2 |
| | PCR | TCAAA | | | |
| CXCR4 | ChIP- | CAGTAGAGACACTGA | 3 | TTGGAAGCTTGGCCCTACTT | 4 |
| | PCR | GGCCC | | | |
| endothelin- | ChIP- | TGTCTGGGGCTGGAAT | 5 | GACTTGGACAGCTCTCTGCC | 6 |
| 1 | PCR | AAAG | | | |
| DLL1 | ChIP- | GCATGGCTAATGAGA | 7 | CATTGAGAGGAGGGTTTGGA | 8 |
| | PCR | TGCAA | | | |
| FZD7 | ChIP- | GCCAATCAGAAAACG | 9 | GCAACCATTTGATCCTCCAT | 10 |
| | PCR | CTACC | | | |
| GAPDH | ChIP- | CGGCTACTAGCGGTTT | 11 | AAGAAGATGCGGCTGACTGT | 12 |
| | PCR | TACG | | | |
| GATA2 | ChIP- | CTGTGCCCAGGTAACC | 13 | CTCGCTCGGTGAGTTTCTTC | 14 |
| | PCR | AAAT | | | |
| GATA6 | ChIP- | GGATGAGAACGGTTT | 15 | TTGTGAACTTGTGGCTCCTG | 16 |
| | PCR | CTGGA | | | |
| HAND1 | ChIP- | GGCAAGGCTGAAAAT | 17 | GATAGCCACTCCCCCTTTTC | 18 |
| | PCR | GAGAC | | | |
| ICAM2 | ChIP- | CTGCCTGGAGGGAGA | 19 | AAGATCTCAGATAATGAGAG | 20 |
| | PCR | TGGT | | GAAATGC | |
| MSX2 | ChIP- | AGGCCTCTAATCCTCG | 21 | CCCTCTCGTTTGCAAGTCTC | 22 |
| | PCR | AAGC | | | |
| MYOD1 | ChIP- | CCCTCTTTCACGGTCT | 23 | GGGTAGAGCGGCTGTAGAAA | 24 |
| | PCR | CACT | | | |
| NANOG | ChIP- | GATTTGTGGGCCTGAA | 25 | GGAAAAAGGGGTTTCCAGAG | 26 |
| | PCR | GAAA | | | |
| PAX6 | ChIP- | TCCTGGGAAGGAGAC | 27 | GAGGTCAGGCTTCGCTAATG | 28 |
| | PCR | AGAGA | | | |
| SOX1 | ChIP- | CAAGTGGTTTGTGCAT | 29 | GACGGAGAGGAATTCAGACG | 30 |
| | PCR | CAGG | | | |
| vWF | ChIP- | TGGGCGGCACCATTGT | 31 | CATACCTTCCCCTGCAAATGA | 32 |
| | PCR | | | | |

| | |
|---|---|
| CD31/PECAM1 | Kanki Y, Kohro T, Jiang S, Tsutsumi S, Mimura I, |
| | Suehiro J, et al. Epigenetically coordinated GATA2 |
| | binding is necessary for endothelium-specific endomucin |
| | expression. EMBO J. 2011; 30(13): 2582-95. |
| CXCR4 | Fraineau S, Palii CG, McNeill B, Ritso M, Shelley WC, |
| | Prasain N, et al. Epigenetic Activation of Pro-angiogenic |
| | Signaling Pathways in Human Endothelial Progenitors |
| | Increases Vasculogenesis. Stem cell reports. |
| | 2017; 9(5): 1573-87. |
| endothelin-1 | Kanki Y, Kohro T, Jiang S, Tsutsumi S, Mimura I, |
| | Suehiro J, et al. Epigenetically coordinated GATA2 |
| | binding is necessary for endothelium-specific endomucin |
| | expression. EMBO J. 2011; 30(13): 2582-95. |
| DLL1 | Fraineau S, Palii CG, McNeill B, Ritso M, Shelley WC, |
| | Prasain N, et al. Epigenetic Activation of Pro-angiogenic |
| | Signaling Pathways in Human Endothelial Progenitors |
| | Increases Vasculogenesis. Stem cell reports. |
| | 2017; 9(5): 1573-87. |
| FZD7 | Fraineau S, Palii CG, McNeill B, Ritso M, Shelley WC, |
| | Prasain N, et al. Epigenetic Activation of Pro-angiogenic |
| | Signaling Pathways in Human Endothelial Progenitors |
| | Increases Vasculogenesis. Stem cell reports. |
| | 2017; 9(5): 1573-87. |
| GAPDH | Pan G, Tian S, Nie J, Yang C, Ruotti V, Wei H, et al. |
| | Whole-genome analysis of histone H3 lysine 4 and lysine |
| | 27 methylation in human embryonic stem cells. Cell Stem |
| | Cell. 2007; 1(3): 299-312. |
| GATA2 | Pan G, Tian S, Nie J, Yang C, Ruotti V, Wei H, et al. |
| | Whole-genome analysis of histone H3 lysine 4 and lysine |
| | 27 methylation in human embryonic stem cells. Cell Stem |
| | Cell. 2007; 1(3): 299-312. |
| GATA6 | Pan G, Tian S, Nie J, Yang C, Ruotti V, Wei H, et al. |
| | Whole-genome analysis of histone H3 lysine 4 and lysine |
| | 27 methylation in human embryonic stem cells. Cell Stem |
| | Cell. 2007; 1(3): 299-312. |
| HAND1 | Pan G, Tian S, Nie J, Yang C, Ruotti V, Wei H, et al. |
| | Whole-genome analysis of histone H3 lysine 4 and lysine |
| | 27 methylation in human embryonic stem cells. Cell Stem |
| | Cell. 2007; 1(3): 299-312. |
| ICAM2 | Kanki Y, Kohro T, Jiang S, Tsutsumi S, Mimura I, |
| | Suehiro J, et al. Epigenetically coordinated GATA2 |
| | binding is necessary for endothelium-specific endomucin |
| | expression. EMBO J. 2011; 30(13): 2582-95. |
| MSX2 | Pan G, Tian S, Nie J, Yang C, Ruotti V, Wei H, et al. |
| | Whole-genome analysis of histone H3 lysine 4 and lysine |
| | 27 methylation in human embryonic stem cells. Cell Stem |
| | Cell. 2007; 1(3): 299-312. |
| MYOD1 | Fraineau S, Palii CG, McNeill B, Ritso M, Shelley WC, |
| | Prasain N, et al. Epigenetic Activation of Pro-angiogenic |
| | Signaling Pathways in Human Endothelial Progenitors |
| | Increases Vasculogenesis. Stem cell reports. |
| | 2017; 9(5): 1573-87. |
| NANOG | Pan G, Tian S, Nie J, Yang C, Ruotti V, Wei H, et al. |
| | Whole-genome analysis of histone H3 lysine 4 and lysine |
| | 27 methylation in human embryonic stem cells. Cell Stem |
| | Cell. 2007; 1(3): 299-312. |
| PAX6 | Pan G, Tian S, Nie J, Yang C, Ruotti V, Wei H, et al. |
| | Whole-genome analysis of histone H3 lysine 4 and lysine |
| | 27 methylation in human embryonic stem cells. Cell Stem |
| | Cell. 2007; 1(3): 299-312. |
| SOX1 | Pan G, Tian S, Nie J, Yang C, Ruotti V, Wei H, et al. |
| | Whole-genome analysis of histone H3 lysine 4 and lysine |
| | 27 methylation in human embryonic stem cells. Cell Stem |
| | Cell. 2007; 1(3): 299-312. |
| vWF | Kanki Y, Koluo T, Jiang S, Tsutsumi S, Mimura I, |
| | Suehiro J, et al. Epigenetically coordinated GATA2 |
| | binding is necessary for endothelium-specific endomucin |
| | expression. EMBO J. 2011; 30(13): 2582-95. |

Western blotting. Cells were collected from either primed (E8 media on vitronectin-coated plates) or naïve (LIF-3i/MEF plates) conditions with Enzyme-Free Cell Dissociation Buffer (Gibco, 13151-014). Cells were washed in PBS and pelleted. Cell pellets were lysed in 1×RIPA buffer (ThermoFisher Scientific, 89900), 1 mM EDTA, 1× Protease Inhibitor (ThermoFisher Scientific, 78430), and quantified using the Pierce bicinchoninic acid (BCA) assay method (ThermoFisher Scientific). 25 μg of protein per sample was loaded on a 4-12% NuPage Gel (ThermoFisher Scientific, NP0336) according to manufacturer's recommendations. The gel was transferred using the iBlot2 (Life Technologies), blocked in Tris-buffered saline (TBS), 5% non-fat dry milk (Labscientific), 0.1% Tween-20 (TBS-T) for 1 hour, and incubated overnight at 4° C. in with anti-phosphorylated-STAT3 primary antibody (Cell Signaling, 9145) according to manufacturer's protocols. Membranes were rinsed 3 times in TBS-T, incubated with horseradish peroxidase (HRP)-linked goat anti-rabbit secondary antibody (Cell Signaling, 7074) for 1 hour at room temperature, rinsed 3 times, and developed using Pierce ECL Substrate (ThermoFisher Scientific, 32106). Chemiluminescence detection was imaged using an Amersham Imager 600 (Amersham). Anti-actin antibody staining was performed for each membrane as a loading control. Quantitative densitometry was performed on all Western blot images presented in this study using ImageJ software. Semi-quantitative densitometry was measured using the ImageJ software and normalized to actin controls.

Flow cytometry analysis of vascular differentiations and FACS purification of CD31⁺CD146⁺ endothelial-pericytic VP populations. Recipes for all differentiation reagents, antibodies, and PCR primers were previously described and also summarized in Table 1. For flow cytometry analysis of vascular differentiations, cells were washed once in PBS, and enzymatically digested with 0.05% trypsin-EDTA (5 min, 37° C.), neutralized with FCS, and cell suspensions were filtered through a 40 μm cell-strainer (Fisher Scientific, Pittsburgh, Pa.). Cells were centrifuged (200 g, 5 min, room temperature) and re-suspended in staining buffer (EBM alone or 1:1 EMG2:PBS). Single cell suspensions (<1×10⁶ cells in 100 μL per tube) were incubated for 20 min on ice with directly conjugated mouse monoclonal anti-human antibodies and isotype controls. Cells were washed with 3 mL of PBS, centrifuged (300 g, 5 min, room temperature), and resuspended in 300 μL of staining buffer prior to acquisition. Viable cells were analyzed (10,000 events acquired for each sample) using the BD CellQuest Pro analytical software and FACSCalibur™ flow cytometer (BD Biosciences). All data files were analyzed using Flowjo analysis software (Tree Star Inc., Ashland, Oreg.).

FACS of primed vs. naive VP populations was performed at the Johns Hopkins FACS Core Facility with a FACS Aria III instrument (BD Biosciences, San Jose, Calif.). Cell suspensions from APEL vascular differentiations were incubated with mouse anti-human CD31-APC (eBioscience, San Diego, Calif.) and CD146-PE (BD Biosciences) antibodies for 30 min on ice, and FACS-purified for high CD31 and CD146 expression, plated onto fibronectin-coated plates in EGM2, and expanded to 80-90% confluency for 7-9 days prior to in vitro analyses or in vivo injections into the eyes of I/R-treated NOG mice.

Vascular functional assays. The methods for endothelial Dil-acetylated-LDL uptake assays, Matrigel tube quantitation assays, EdU proliferation assays, β-galactosidase senescent assays were all described previously⁷, and are summarized below briefly. For Dil-Acetylated-Low Density Lipoprotein (Dil-Ac-LDL) uptake assays, FACS-purified CD31⁺CD146⁺ primed vs naïve VP populations were expanded in EGM2 medium ˜7 days to 60 to 70% confluency on fibronectin pre-coated 6-well plates (1-1.5×10⁵ cells/well) prior to Dil-Ac-LDL uptake assays (Life Technologies, Cat No. L-3484). Fresh EGM2 medium supplemented with 10 μg/mL Dil-Ac-LDL, was switched before assays, and incubated for 4 hours at 37° C. Cells were washed in PBS and Dil-Ac-LDL-positive cells imaged with a Nikon Eclipse Ti-U inverted microscope (Nikon Instruments Inc., Melville, N.Y.) and Eclipse imaging software. Cells were also harvested with Accutase (5 min, 37° C.) and Dil-Ac-LDL⁺ cells quantitated by flow cytometry.

In vitro vascular functionality of primed DVP vs N-DVP was determined with quantitative Matrigel vascular tube-forming assays as previously described⁷. Briefly, MACS-purified CD31⁺CD146⁺ isogenic DVP were expanded in EGM2 on fibronectin-coated (10 μg/mL). tissue culture plates. Adherent cells were treated with Accutase for 5 min, and collected into single cell suspensions. Primed DVP or N-DVP cells were transferred in 48-well plates (2×10⁵ cells/well in EGM2 medium) pre-coated with Matrigel (Corning, #356237, 200 μL/well). The next day, multiple phase contrast pictures of vascular tube formations were imaged with an inverted Eclipse Ti-u Nikon microscope (Nikon Instruments Inc., Melville, N.Y.) and Eclipse imaging software without overlapping the imaged regions. All the vascular tubes formed by VP, DVP, and N-DVP were measured by NIS-Elements imaging software. Statistical comparisons were performed with unpaired t-tests using Prism (GraphPad Software, San Diego, Calif.).

For senescence assays, naïve vs primed VP populations were plated onto fibronectin (10 μg/mL)-coated 6-well tissue culture plates, and VP were expanded in EGM2 for up to 30 days (3-6 passages), and senescent cells were assayed for acidic senescence-associated 8-galactosidase activity. Cells were grown to ˜60-80% confluency in 12-well fibronectin-coated plates prior to analysis. Cells were fixed in 2% paraformaldehyde and Q-galactosidase activity was quantified by detecting hydrolysis of the X-gal substrate by colorimetric assay as per manufacturer's protocol for detection of senescent cells. (Cell Signaling Technology, Danvers, Mass.). Nuclei were counterstained using the fluorescent dye Hoechst 33342 (BD Biosciences). Total number of Hoechst⁺ cells and blue X-Gal⁺ senescent cells were automatically enumerated using an inverted Eclipse Ti-u Nikon microscope and the Object Count component of the NIS Elements software. For each sample, 2 individual wells were photographed at five independent locations using a 20× objective.

Transmission Electron Microscopy (TEM) of DVP and N-DVP. Primed vs naïve VP were plated onto fibronectin (10 μg/mL) coated Labtek chambers, culture expanded in EGM2, and fixed for TEM as previously described at the Wilmer Microscopy Core⁷. Sections were imaged with a Hitachi H7600 TEM at 80 KV (Gaithersburg, Md.) and a side mount AMT CCD camera (Woburn, Mass.).

NCS DNA damage response assays. Primed DhiPSC and N-DhiPSC isogenic (same lines at same passage) were simultaneously differentiated in parallel into DVP and N-DVP using APEL medium, as described above. CD31⁺CD146⁺ VP cells were expanded in EGM2 (3 passages) onto fibronectin-coated (10 μg/mL) 6-well plates (for Western blot analysis), or alternatively the last passage was transferred onto 8-well Nunc Labtek II chamber slides for immunostaining. To induce DNA damage, expanded DVP and N-DVP cells were incubated for 5 hours in EGM2 supplemented with 100 ng/mL of the radiomimetic agent neocazinostatin (NCS, Sigma). Untreated DVP and N-DVP cells were analyzed in parallel as controls. Western blot analysis was performed as described above. For detection of phosphorylated H2AX by immunofluorescence, VP cells were fixed for 10 minutes using 1% paraformaldehyde in PBS. For immunofluorescent staining of chambered slides, fixed cells were blocked for non-specific staining and permeabilized using a blocking solution consisting of PBS, 5% goat serum (Sigma) and 0.05% Tween 20 (Sigma). Samples were incubated overnight at 4° C. with a rabbit anti-human phospho-H2AX antibody (Cell Signaling, #9718) diluted (1:200) in blocking solution. The next day, VP cells were washed (Dako wash buffer, Dako) and incubated for 2 hours at room temperature with a biotinylated goat anti-rabbit secondary antibody (Dako, 1:500 in blocking solution). Cells were washed 3 times and incubated for 30 minutes with streptavidin Cy3 (Sigma, 1:500). All samples were sequentially washed and incubated with a mouse monoclonal anti-human CD31 (Dako, M0823, 1:100) and Alexa488-conjugated goat anti-mouse secondary antibody (ThermoFisher, 1:100), both for 1 hour at room temperature. Finally, slides were washed in PBS and incubated with DAPI (1:2000) for 5 minutes at room temperature for nuclear staining. Slides were mounted using the Prolong Gold anti-fade mounting reagent (ThermoFisher) and cured overnight. For each condition, 5 to 6 independent frames were captured for the Cy3, Alexa488 and DAPI channels using a 20× objective and a LSM510 Meta confocal microscope (Carl Zeiss Inc., Thornwood, N.Y.) in the Wilmer Eye Institute Imaging Core Facility. Quantification of phospho-H2AX⁺ foci within DAPI⁺ nuclei of CD31⁺ VP was performed using the NIS-Elements software. Briefly, thresholds and masks were sequentially created for the Alexa488 (CD31) and DAPI channels to limit the analysis to nuclei of VP cells. Nuclei were further defined using the size/area and circularity parameters. Each individual CD31⁺ nucleus was characterized as a single object using the “object count” function. Finally, the number of foci per nucleus was determined by counting the number of objects in the Cy3 channel. A total of 128 to 165 nuclei were analyzed for each condition (primed vs. naive±NCS). Statistical comparisons of the distribution of number of phosphor-H2Ax+ foci per nuclei between VP populations were assessed by Chi-square test (z-test) using Graphpad Prism.

Ocular I/R Injury and VP Injections into NOD/Shi-scid/IL-2Rγ^{null} (NOG) eyes. The I/R ocular injury model was previously described 7. Briefly, six- to eight-week old male NOG mice (Johns Hopkins Cancer Center Animal Facility) were subjected to high intraocular pressure to induce retinal ischemia-reperfusion injury. Mice were deep anesthetized by intraperitoneal (IP) injection of ketamine/xylazine (50 mg/kg ketamine+10 mg/kg xylazine in 0.9% NaCl). The pupils were dilated with 2.5% phenylephrine hydrochloride ophthalmic solution (AK-DILATE, Akorn, Buffalo Glove, Ill.) followed by 0.5% tetracaine hydrochloride ophthalmic topical anesthetic solution (Phoenix Pharmaceutical, St. Joseph, Mo.). The anterior chamber of the eye was cannulated under microscopic guidance (OPMI VISU 200 surgical microscope, Zeiss, Gottingen, Germany) with a 30-gauge needle connected to a silicone infusion line providing balanced salt solution (Alcon Laboratories, Fort Worth, Tex.); avoiding injury to the corneal endothelium, iris, and lens. Retinal ischemia was induced by raising intraocular pressure of cannulated eyes to 120 mmHg for 90 min by elevating the saline reservoir. Ischemia was confirmed by iris whitening and loss of retinal red reflex. Anesthesia was maintained with two doses of 50 μL intramuscular ketamine (20 mg/mL) for up to 90 min. The needle was subsequently withdrawn, intraocular pressure normalized, and reperfusion of the retinal vessels confirmed by reappearance of the red reflex. The contralateral eye of each animal served as a non-ischemic control. Antibiotic ointment (Bacitracin zinc and Polymyxin B sulfate, AK-Poly-Bac, Akron) was applied topically. Two days later, MACS-purified and expanded human DVP and N-DVP were injected into the vitreous body (50,000 cells in 2 μL/eye), using a micro-injector (PLI-100, Harvard Apparatus, Holliston, Mass.).

Immunofluorescence staining of flat whole-mounted NOG mouse retinae. Human cell engraftment into NOG mouse retinae were detected directly with anti-human nuclear antigen (HNA) immunohistochemistry with murine vascular marker co-localization (murine CD31 and collagen IV) using anti-murine CD31 and anti-murine collagen IV antibodies. Animals were euthanized for retinal harvests and HNA-positive cell quantitation at 1, 3, and 4 weeks following human VP injection (2 days post-I/R injury). After euthanasia, eyes were enucleated, cornea and lens were removed, and the retina was carefully separated from the choroid and sclera. Retinae were fixed in 2% paraformaldehyde in TBS for overnight at 4° C., and permeabilized via incubation with 0.1% Triton-X-100 in TBS solution for 15 min at 4° C. Following thorough TBS washes, free floating retinas were blocked with 2% normal goat serum in TBS with 1% bovine serum albumin and incubated overnight at 4° C. in primary antibody solutions: rabbit anti-mouse Collagen IV (AB756P, Millipore, 1:100) and/or rat anti-mouse CD31 (550274, BioSciences, 1:50) in 0.1% Triton-X-100 in TBS solution (to label basement membrane and EC of blood vessels, respectively). On the next day, retinae were washed with TBS, and incubated with secondary antibodies for 6 hours at 4° C. A goat anti-rabbit Cy3-conjugated secondary antibody (Jackson Immuno Research, #111-165-003, 1:200) was used to detect collagen IV primary antibody, and a goat anti-rat Alexafluor-647-conjugated secondary antibody (Invitrogen, #A21247, 1:200) was used to detect the anti-CD31 primary antibody. Human cells were detected using directly Cy3-conjugated anti-HNA (Millipore, MAB1281C3, 1:100). After washing in TBS, flat mount retinas were imaged with confocal microscopy (LSM510 Meta, Carl Zeiss Inc., Thornwood, N.Y.) at the Wilmer Eye Institute Imaging Core Facility.

Immunofluorescent confocal microscopy and quantitation of human cell vascular engraftment in murine retinae. For quantification of HNA⁺ cells in the superficial layers of whole retinae, whole mount retinas were prepared from the eyes of animals at 1, 3 or 4 weeks following intra-vitreal transplantation of human cells (50,000 primed DVP or N-DVP cells per eye) following I/R injury. Non-I/R injured eyes and control PBS-injected eyes were also analyzed as controls. Images were acquired with ZEN software using a 10× objective and a LSM510 Meta confocal microscope. For each individual eye, the entire retina was tile-scanned and stitched (7×7 frames, 10% overlapping).

For human HNA⁺ cell quantification analysis, photomicrographs were processed using the Fiji distribution of imageJ. Briefly, a region of interest was created using the DAPI channel and the “magic wand” function to conservatively delineate the whole retina and exclude from the analysis the limited background at the edges of the retina preparation that could be detected in the Cy3 (HNA) channel for some samples. The Cy3 channel was processed with the “smooth” function and a mask was created using by thresholding. The Cy3 channel was further prepared for the “analyze particle” plugin by using standardized sequential corrections that were limited to despeckle, filtering (Minimum) and watersheding. Particle objects corresponding to HNA+ nuclei were automatically counted using fixed size and circularity parameters.

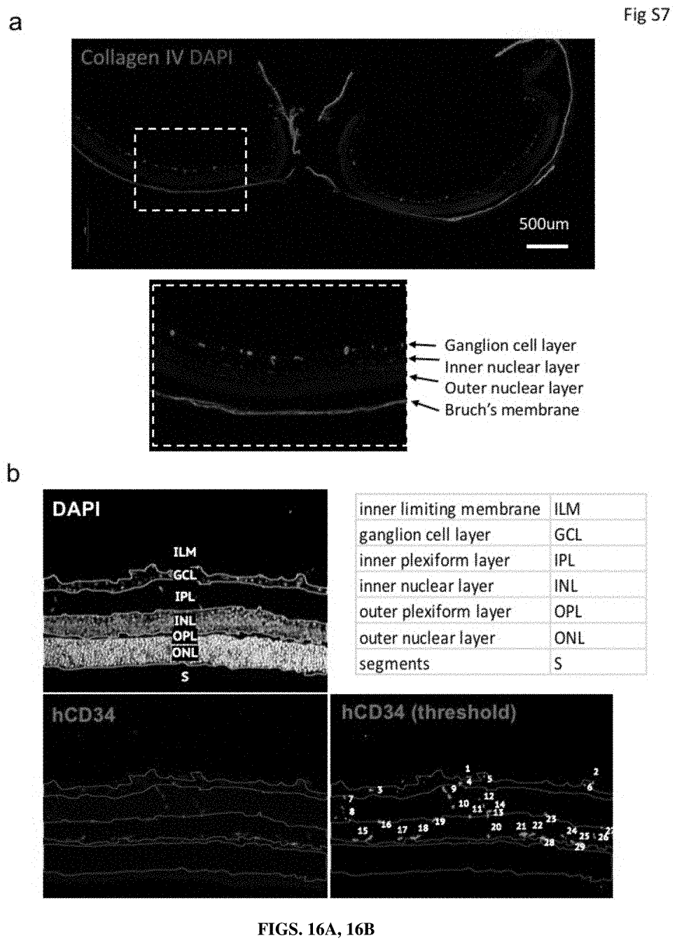

Eyes were also analyzed for quantification of human CD34⁺ or human CD31⁺ blood vessels within defined layers of the mouse retina in some experiments. Briefly, the anterior eye (cornea/iris) was dissected free by a circumferential cut at the limbus. Eyecups were fixed using paraformaldehyde and prepared for cryopreservation by immersion in gradients of sucrose (Lutty et al IOVS 1993, PMID: 7680639). Eyes were hemisected through the optic nerve (FIG. 16A) and the two halves embedded in OCT-sucrose. Serial cryosections (8 μm thickness) were prepared from hemisections that included the retina (FIG. 16A), and stored at −80° C. Equally interspaced microsections [n=11 (E8) and 13 (LIF-3i) for CD34, and n=3 (E8) and 7 (LIF-3i) for CD31 immunostainings]. Retinal sections were sequentially immunostained with either mouse anti-human CD34 (BD Biosciences, clone My10, #347660, 1:50) or mouse anti-human CD31 (Dako, M0823, 1:50) overnight at 4° C. followed by goat anti-mouse Alexa488 (ThermoFisher Scientific, 1:200) for 1 hour at room temperature. Sections were subsequently stained with either rabbit anti-mouse collagen type IV (Millipore, AB756P, 1:200) or rat anti-mouse CD31 (BD Biosciences, 550274, 1:50) for 1 hour at room temperature. Alexa647-conjugated goat-anti rabbit (ThermoFisher Scientific, A-21246; 1:200) or goat anti-rat (ThermoFisher Scientific) secondary antibodies (i.e., conjugated F(ab′)2 fragments) that were highly cross-adsorbed against IgG from other species were subsequently incubated for 1 hour at room temperature. Nuclei were counterstained using DAPI. Negative immunostaining controls in each experiment were conducted and confirmed negative, and consisted of replacing primary antibodies with mouse, rat and rabbit nonimmune IgG (Dako or ThermoFisher Scientific) at the corresponding antibody concentration to verify absence of unspecific antibody binding. Retinal sections were mounted with Prolong Gold anti-fade reagent (ThermoFisher Scientific) and cured overnight in the dark. Images were acquired using a 20× objective with the ZEN software and a LSM510 Meta confocal microscope.

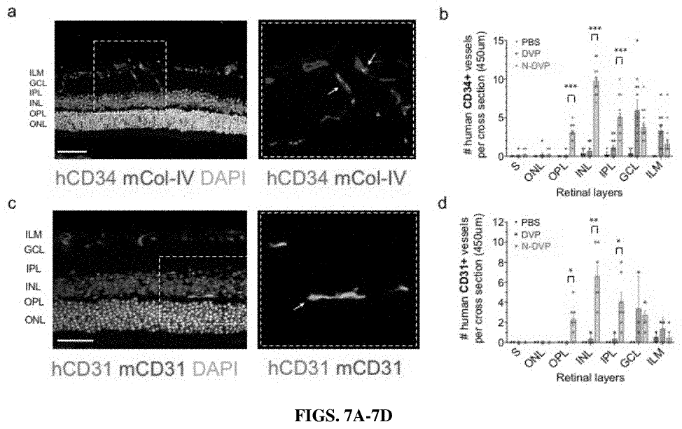

Photomicrographs were further processed for human cell quantification using the Fiji distribution of imageJ (FIG. 16B). Briefly, regions of interest (ROI) were created using the DAPI channel as a template to delineate the GCL, INL, and ONL, the other regions (ILM, IPL, OPL and S) being defined as intercalated around and between the 3 DAPI-defined ROI. Analysis was pursued by processing the Alexa488 (human CD34 or CD31) channel using a sequential series of defined parameters using the “smooth”, “despeckle”, “filter (median)” functions and thresholding. Alexa488⁺ objects were counted within and between ROI using ImageJ. The numbers of human CD34⁺ or CD31⁺ blood vessels were automatically enumerated using the “Analyze particles” plugin within Fiji. Images were captured using a 20× objective (450 μm²). Speckles and non-specific background (<10 pixels) were excluded. Regions delineating the retinal layers were created using the DAPI channel. A smoothened image was segmented by thresholding the CD34 or CD31 signal (pixel values >50). The ImageJ module “Analyze particles” was set to select object with a surface area >5 μm² and each image automatic count was cross-validated by manual counting of threshold images and exclusion of duplicate objects (2 or more objects belonging to blood vessels that were longitudinally cross-sectioned). Human CD34 (FIG. 16B) or human CD31 expression was scored only when the Alexa488⁺ signal was expressed at chimeric human-murine blood vessels that also expressed either murine collagen IV (mColIV) or murine CD31 (mCD31) (FIGS. 7A, 7C). The quantity of human blood vessels detected in murine vessels ranged between 1-13 per image at 20× objectives (FIGS. 7B, 7D).

Quantitative Real-time Polymerase Chain Reaction (qRT-PCR) and Chromatin Immunoprecipitation PCR (ChIP-PCR). The sequences and published reference citations of all PCR primers used in these studies for qRT-PCR and qChIP-PCR are listed in Table 1 above. For qRT-PCR analyses, feeder-dependent LIF-3i hPSC cultures were MEF-depleted by pre-plating onto 0.1% gelatin-coated plates for 1 hour at 37° C., as previously described¹². Samples were sequentially and simultaneously collected from representative hPSC lines in primed (E8), or naïve (LIF-3i; p>3) conditions. Alternatively, genotypic-identical (isogenic) paired samples were prepared from EGM2-expanded primed and naïve VP. Total RNA was isolated from snap-frozen samples using the RNeasy Mini Kit (Qiagen) following the manufacturer's instructions, and quantified using a Nanodrop spectrophotometer (ThermoFisher Scientific). Genomic DNA was eliminated by in-column DNase (Qiagen) digestion. Reverse transcription of RNA (1 μg/sample) was accomplished using the SuperScript VILO cDNA Synthesis Kit (ThermoFisher Scientific) and a MasterCycler EPgradient (Eppendorf). For real-time PCR amplification, diluted (1:20) cDNA samples were admixed to the TaqMan Fast Advanced Master Mix (ThermoFisher Scientific) and Taqman gene expression assays (ThermoFisher Scientific).

Matching isogenic samples were prepared in parallel for ChiP-PCR assays. Isogenic hPSC cultures were expanded using primed (E8) and naïve (LIF-3i/MEF) conditions and analyzed at passages matching RT-PCR analysis. Alternatively, VP cells were prepared from isogenic primed and naïve PSC using the same APEL/EGM2 conditions as the samples prepared for RT-PCR. Cells were collected using Accutase and counted using a Countess cell counter (ThermoFisher Scientific). Feeders were excluded from LIF-3i/MEF samples by pre-plating for 1 hour on gelatin-coated plates and pre-plated samples were re-counted after the pre-plating step. 3×10⁶ cells were allocated per ChTP assay and prepared using the Magna ChIP A/G chromatin immunoprecipitation kit (Millipore). Cells were centrifuged (300 g), supernatant was discarded and cells were fixed for 10 minutes at room temperature by resuspending in 1 mL of PBS, 1% formaldehyde (Affymetrix). Unreacted formaldehyde was quenched using 100 μL 10× Glycine (Millipore). Samples were left at room temperature for 5 minutes, centrifuged (300 g) and washed twice in 1 mL ice-cold PBS. Samples were resuspended in ice-cold PBS containing either 1× Protease Inhibitor Cocktail II (Millipore) or 1× complete Mini protease inhibitor (Roche). Samples were centrifuged at 800 g for 5 minutes, cell pellets were snap-frozen in liquid nitrogen and stored at −80° C. until use for ChTP assay. Cell lysis, homogenization and nuclear extraction of cryopreserved samples were processed using the reagents provided in the Magna ChIP kit and the manufacturer instructions. The isolated chromatin was fragmented using a Diagenode Bioruptor Plus sonication device. Sonication settings (10 cycles, high, 30 s on, 30 s off) were validated in pilot experiments to shear cross-linked DNA to 200-1000 base pairs by agarose gel electrophoresis. The sheared chromatin was centrifuged at 10,000 g at 4° C. for 10 minutes and immediately processed for immunoprecipitation. 1×10⁶ cell equivalent of cross-linked sheared chromatin were prepared according to the kit manufacturer's protocol. Briefly, 1% of sheared chromatin was separated as “input” control. The remaining sample was admixed with 5 μg of immuno-precipitating antibody (Table 1) and protein A/G magnetic beads. Antibodies were substituted with corresponding rabbit or mouse IgG (Table 1) as negative isotype controls using 5% sheared chromatin. The chromatin-antibody-beads mixture was left incubating overnight at 4° C. with agitation. Protein A/G beads were pelleted using a MagJET separation rack (ThermoFisher Scientific) and supernatant was discarded. Protein/DNA complexes were washed and eluted, beads were separated using the MagJET rack and DNA was purified according to the manufacturer's instructions. The immunoprecipitated genomic DNA was amplified using the Power SYBR Green Master Mix (ThermoFisher Scientific) with relevant published primers (Table 1) for GAPDH, GATA2, GATA6, HAND1, NANOG, MSX2, PAX6, SOX1, CD31, vWF, endothelin-1, ICAM2, MYOD1 (Lutty, G. A. Diabetic choroidopathy. Vision Res 139, 161-167, doi:10.1016/j.visres.2017.04.011 (2017)), CXCR4, DLL1, FZD7 and ELP3 using a ViAA7 Real Time PCR System (ThermoFisher Scientific). Specificity of antibodies was validated using the isotype controls and samples were normalized to their corresponding input controls.

Genomic DNA dot-blots of 5-methylcytosine (5MC) and 5-hydroxymethylcytosine (5hMC) CpG methylation. Genomic DNA from isogenic parallel primed (E8) and preplated LIF-3i cultures of representative hiPSC lines was extracted using the DNeasy Blood and tissue Kit (Qiagen) and quantified using a Nanodrop spectrophotometer (ThermoFisher Scientific), as described¹². For each sample, 1.6 μg DNA was diluted in 50 μL of nuclease-free water (Ambion), denatured by adding 50 μL of 0.2M NaOH, 20 mM EDTA and incubating for 10 minutes at 95° C., and neutralized by adding 100 μL 20× Saline-Sodium Citrate SSC hybridization buffer (G Biosciences) and chilling on ice. A series of five 2-fold dilutions (800 ng to 50 ng) and nuclease-free water controls were spotted on a pre-wetted (10×SSC buffer) nylon membrane using a Bio-Dot Microfiltration Apparatus (Bio-Rad). The blotted membrane was air-dried and UV-cross-linked at 1200 J/m² using a UV Stratalinker 1800 (Stratagene). The membrane was blocked in TBST, 5% nonfat dry milk for 1 hour at room temperature with gentle agitation, washed 3 times in TBST, and incubated at 4° C. overnight with rabbit anti-5mC (Cell Signaling, 1:1000) or anti-5hmC (Active Motif, 1:5000) antibodies diluted in TBST, 5% BSA. The membrane was washed 3 times in TBST and incubated for 1 hour at room temperature with HRP-conjugated anti-rabbit secondary antibody (Cell Signaling) diluted 1:1000 in blocking buffer. After 3 washes in TBST, the membrane was treated with Pierce ECL Substrate (ThermoFisher) for chemiluminescent detection with an Amersham Imager 600 (Amersham). After acquisition, the membrane was washed 3 times in H₂O and immersed in 0.1% methylene blue (Sigma), 0.1M sodium acetate stain solution for 10 minutes at room temperature. Excess methylene blue was washed 3 times in water with gentle agitation. Colorimetric detection was achieved using the Amersham Imager 600 (Amersham). 5mC, 5hmC, and methylene blue intensities were quantified by ImageJ software.

Statistics. Statistical significance was determined using statistical graphing software (Prism GraphPad) using two-tailed t tests (between individual groups), or 1-way analysis of variance (e.g., analysis of variance-Eisenhart method with Bonferroni correction) for statistical testing of ≥3 groups. For smaller, non-Gaussian-distributed sample sizes (n<10), nonparametric (Mann-Whitney) tests were performed. P values of at least <0.05 were considered significant.

Expression and CpG methylation arrays, RNA-Sequencing (RNA-Seq), and bioinformatic analyses. The (Illumina, San Diego, Calif. gene expression arrays (Illumina Human HT-12 Expression BeadChip) and Infinium 450K CpG methylation raw array data analyzed in these studies were published previously and available at Gene Expression Omnibus under accession numbers GSE65211 and GSE65214, respectively, and processed as previously described¹². Gene specific enrichment analysis (GSEA) of expression arrays was conducted as described⁶⁶. The bioinformatics method for calculating crossplots of differential promoter CpG methylation beta values vs. corresponding differential gene expression was previously described¹².

For RNA-Seq studies, strand specific mRNA libraries were generated using the NEBNext Ultra II Directional RNA library prep Kit for Illumina (New England BioLabs #E7760), mRNA was isolated using Poly(A) mRNA magnetic isolation module (New England BioLabs #E7490). Preparation of libraries followed the manufacturer's protocol (Version 2.2 05/19). Input was 1 μg. and samples were fragmented for 15 min for RNA insert size of ˜200 bp. The following PCR cycling conditions were used: 98° C. 30 s/8 cycles: 98° C. 10 s, 65° C. 75 s/65° C. 5 min. Stranded mRNA libraries were sequenced on an Illumina HiSeq4000 instrument using 47 bp paired-end dual indexed reads and 1% of PhiX control. mRNA sequencing depth ranged from 30-100M reads. Reads were aligned to GRCh38 using STAR version 2.7.2b⁶⁷ with the following options --readFilesCommand zcat --outSAMtype BAM Unsorted SortedByCoordinate --quantMode TranscriptomeSAM GeneCounts -outFileNamePrefix. Summarized experiment objects were obtained using the gtf file Homo_sapiens.GRCh38.97.gtf and the following command from the Bioconductor package ‘GenomicAlignments’: summarizeOverlaps (features=exonsByGene, reads=bamfiles, mode=“Union”, singleEnd=FALSE, ignore.strand=FALSE, fragments=TRUE. Differential expression analysis and statistical testing was performed using DESeq2 software⁶⁸. The NIH Gene Expression Omnibus has issued the accession number GSE141639 for RNA-Seq data in this manuscript. The GEO-supplied link for access is: https://www.ncbi.nlm.nih.gov/geo/query/acc.cgi?acc=GSE141639.

Results

LIF-3i naïve reversion of conventional, primed hiPSC lines significantly improved their multi-lineage differentiation potency. Culture of conventional hiPSC with a small molecule cocktail of LIF, the tankyrase inhibitor XAV939, the GSK3β inhibitor CHIR99021, and the MEK inhibitor PD0325901 (LIF-3i) conferred a broad repertoire of normal, non-diseased hiPSC^{12,13} with molecular and biochemical characteristics that are unique to naïve pluripotency, including increased phosphorylated STAT3 signaling, decreased ERK phosphorylation, global 5-methylcytosine CpG hypomethylation, genome-wide CpG demethylation at ESC-specific gene promoters, and dominant distal OCT4 enhancer usage^{12,13} LIF-3i-reverted N-hiPSC maintained normal karyotypes, and were devoid of systematic loss of imprinted CpG patterns or irreversible demethylation defects reported in other naïve reversion systems, and that were attributed to prolonged culture with MEK inhibitors²²⁻²⁴.

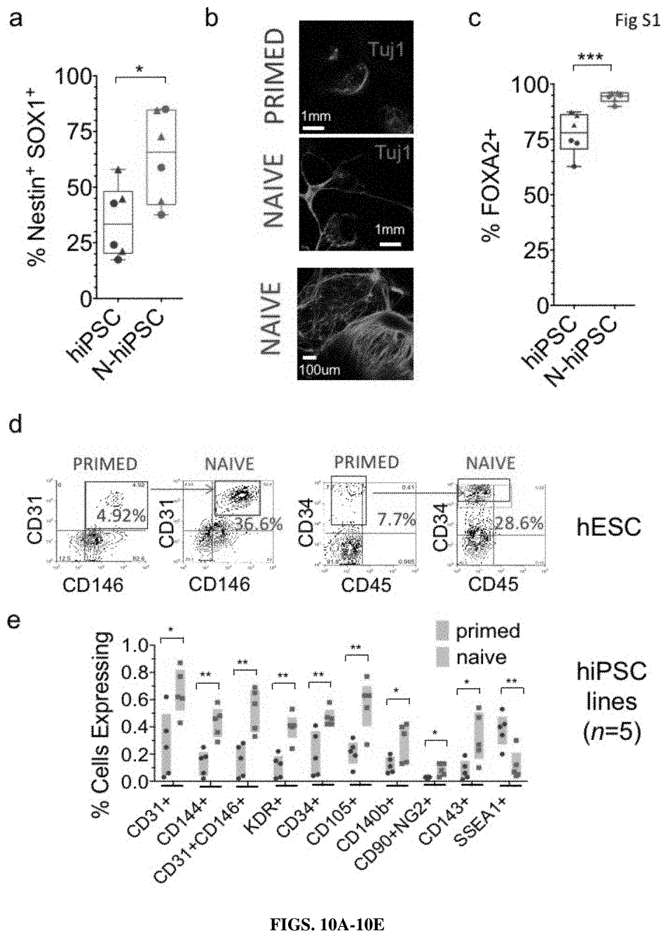

LIF-3i reversion of a broad repertoire of non-diseased conventional, primed hiPSC and hESC was reported to decreased lineage-primed gene expression, and diminished the interline variability of directed differentiation typically observed amongst independent primed, conventional hPSC lines^{12,13}. For further validation, a cohort of isogenic (genotypically-identical) naïve vs. primed, conventional normal (non-diabetic) cord blood (CB)- and fibroblast-derived hiPSC and hESC lines were differentiated in parallel using established multi-lineage differentiation protocols (FIGS. 10A-10E). In contrast to reports of difficult directed differentiations of human naïve states, or requirement for culture back to a primed pluripotent state^{25,26}, LIF-3i-reverted N-hiPSC differentiated directly to all three germ layer lineages with significantly higher efficiencies and more rapid kinetics of differentiation than their isogenic primed, conventional hiPSC counterparts. This high multi-lineage differentiation potency of N-hPSC did not require an additional ‘re-priming’ culture step. For example, in identical neural induction medium conditions, N-hiPSC produced significantly higher levels of Nestin⁺SOX1⁺ neural progenitors than their isogenic primed hiPSC counterparts (FIG. 10A). Additionally, neural ectodermal progenitor cells from N-hiPSC more efficiently differentiated into elongated neurites expressing Tuj1 than their isogenic primed hiPSC counterparts (FIG. 10B). Similarly, LIF-3i N-hiPSC vs their isogenic primed hiPSC counterparts were directly differentiated in parallel cultures into definitive endoderm; N-hiPSC generated significantly higher quantities of FOXA2⁺ progenitors at levels surpassing their isogenic primed hiPSC controls (FIG. 10C). Finally, LIF-3i-reverted N-hiPSC and hESC differentiated with significantly higher efficiencies than their isogenic primed hESC and hiPSC controls into vascular mesoderm (e.g., CD31⁺CD146⁺, KDR⁺, CD140b⁺, CD34⁺, and CD143⁺ VP), and regardless of hiPSC genotypic background or donor source (i.e., fibroblast or cord blood derived; n=5) (FIGS. 10D, 10E). The improved differentiation performance of N-hiPSC erased the poor efficiency and interline variability of vascular lineage differentiation routinely observed between independent conventional fibroblast-derived hiPSC lines.

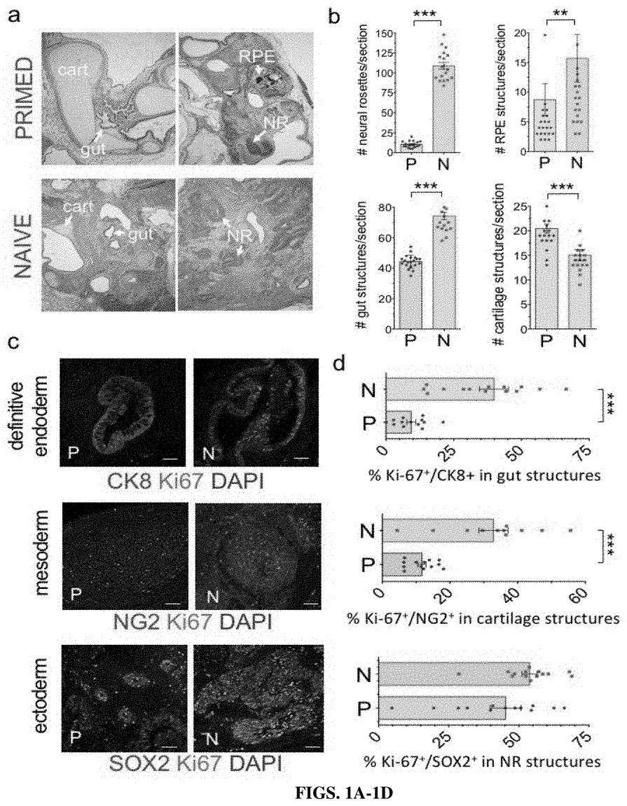

To further validate the functional pluripotency of normal (non-diabetic) fibroblast-derived N-hiPSC in vivo, multi-lineage differentiation performance in teratoma assays was performed (FIGS. 1A-1D). Gross histological analysis of eight-week old teratomas from primed and naïve normal (non-diabetic) fibroblast-hiPSC demonstrated that although both robustly generated lineages of all three germ layers, there were marked quantitative differences between isogenic primed and naive-reverted fibroblast-hiPSC in generating teratoma organoid structures. Whereas primed fibroblast-hiPSC produced well-formed cystic teratomas with strong bias toward mesodermal cartilage differentiation (FIG. 1), N-hiPSC-derived teratomas generated more homogenous and robust distribution of multiple structures from all three germ layer lineages throughout the tissues, and with significantly greater number per cross section of endodermal (gut) structures, neuro-ectodermal (neural rosettes, retinal pigmented epithelial) structures, and mesodermal (cartilage) structures (FIGS. 1A, 1). Furthermore, fibroblast-derived N-hiPSC teratomas (n=3) generated proliferating multi-lineage organoid structures with significantly higher proliferative indices (e.g., 30-50% Ki67⁺ in CK8⁺ gut endodermal, NG2⁺ mesodermal cartilage structures) than their isogenic primed fibroblast-hiPSC counterpart (FIGS. 1C, 1D).

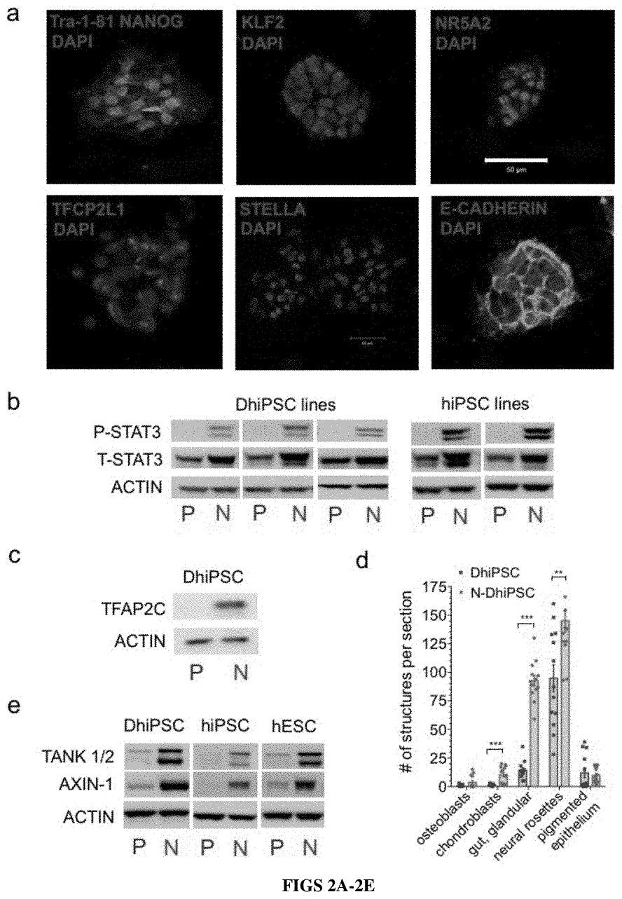

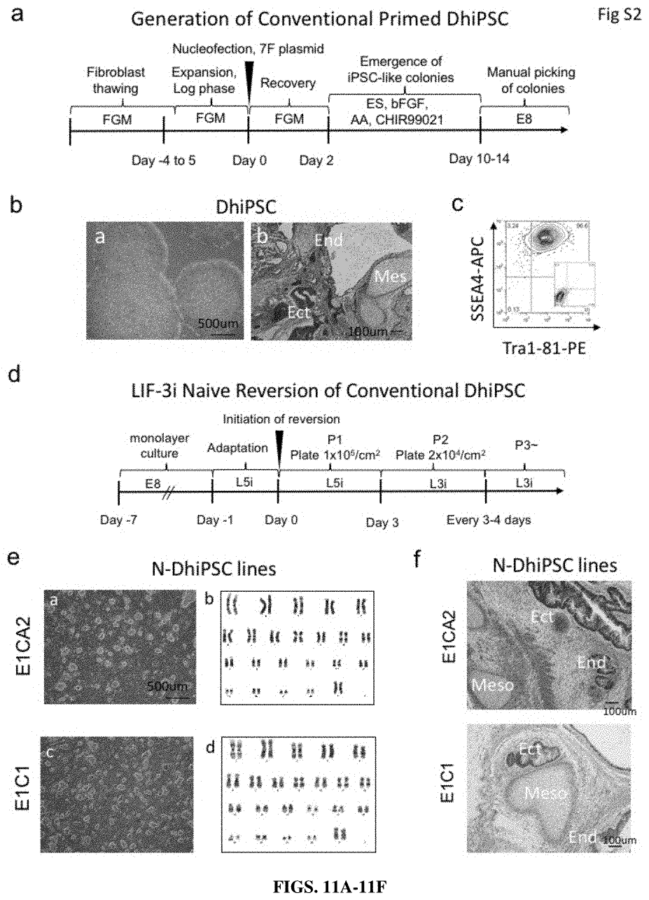

Reprogramming of skin fibroblasts of a type-1 diabetic donor to conventional DhiPSC and subsequent naïve reversion to N-DhiPSC. To test the therapeutic potential of embryonic VP derived from vascular disease-affected fibroblast-hiPSC, we generated several independent conventional SSEA4⁺TRA-1-81⁺ DhiPSC lines from type-1 diabetic donor skin fibroblasts using a modified version of a non-integrative 7-factor episomal reprogramming system²⁷⁻²⁹ (FIGS. 11A-11C). Reversion of conventional, primed normal non-diabetic hiPSC or DhiPSC with the LIF-3i naïve culture system that included the tankyrase/PARP inhibitor XAV939 (FIG. 11D), resulted in changes from flattened (FIG. 11B) to dome-shaped SSEA4⁺TRA-1-81⁺ N-DhiPSC colony morphologies (FIG. 11E). The primed to naive transition was accompanied by activation of protein expressions of a panoply of naïve-specific pluripotency factors (FIG. 2A) (e.g., NANOG, KLF2, NR5A2, TFCP2L1, STELLA/DPPA3, and E-CADHERIN), as well as naïve ESC-specific proteins, that included phosphorylated STAT3 and TFAP2C³¹ (FIGS. 2B, 2C). All N-DhiPSC lines possessed normal karyotypes (FIG. 11E), and generated robust tri-lineage teratomas with significantly higher differentiation performances of organoid structures representing all three germ layers than their primed isogenic counterparts (FIGS. 2D, 11F), and with comparable efficiencies to non-diabetic N-hiPSC (FIGS. 1A-1D).

To validate the effects of XAV939 inhibition of tankyrase-PARP activity in DhiPSC, proteolytic inhibition was verified for key proteins targeted by tankyrase PARylation, including AXIN1 (which synergizes with the GSK3β inhibitor to stabilize the activated β-catenin complex²⁰), and tankyrase 1 (PARP-5a) and tankyrase 2 (PARP-5b) proteins (which self-regulate their own proteolysis by auto-PARylation)³⁰. Accordingly, chemical inhibition of their degradation resulted in high accumulated levels of tankyrases ½ and AXIN1 in LIF-3i-reverted N-DhiPSC that was comparable to non-diabetic fibroblast- and non-diabetic cord blood (CB)-derived hiPSC lines (FIG. 2E).

LIF-3i naïve reversion improved the efficiency of vascular lineage differentiation of diabetic donor-derived conventional hiPSC lines. A FACS-purified CD31⁺CD146⁺CXCR4⁺ embryonic VP population was identified, which differentiated from conventional (non-diabetic) hiPSC that possessed both endothelial and pericytic functionalities⁷. It was demonstrated that ischemia-damaged retinal vasculature could be repaired by transplantation of these hiPSC-derived CD31⁺CD146⁺ embryonic VP generated from conventional hiPSC, and that they possessed prolific endothelial-pericytic differentiation potential and engrafted and rescued degenerated retinal vasculature following ocular ischemia-reperfusion (I/R) injury.

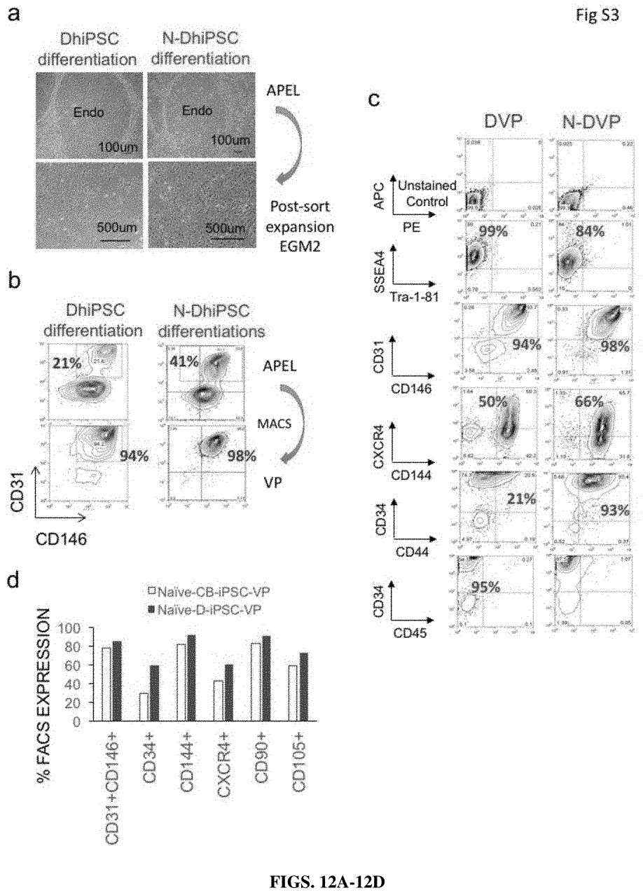

An optimized isogenic primed vs naïve hiPSC version of the original VP differentiation system was used⁷ (FIG. 3A), and similar to results obtained for non-diabetic hiPSC^{32,33} (FIGS. 10D, 10E), it was found that CD31⁺CD146⁺ DVP cells were more efficiently generated from N-DhiPSC, and with more rapid differentiation kinetics than their primed, conventional isogenic DhiPSC counterparts (FIGS. 3B, 3C; 12A, 12B). Kinetic analyses of parallel vascular differentiation cultures of N-DhiPSC vs primed isogenic DhiPSC revealed higher expressions of vascular surface markers (e.g., CD34, CD31, CD146, CD144), and a more rapid decrease of pluripotency markers (e.g., SSEA4, TRA-1-81) in these naïve diabetic VP (N-DVP). Naïve reversion permitted comparable efficiencies of generation of CD31⁺CD146⁺CXCR4⁺ VP populations regardless of conventional hiPSC donor source (i.e., diabetic or non-diabetic). For example, naïve-reverted fibroblast-derived N-DhiPSC and non-diabetic cord blood (CB)-derived N-CB-hiPSC lines both generated similar efficiencies of VP, despite a previously reported higher VP differentiation efficiency of conventional CB-hiPSC compared to conventional fibroblast-derived hiPSC⁷ (FIG. 3C). Following MACS enrichment of CD31⁺CD146⁺ VP populations from vascular differentiation cultures of both non-diabetic and diabetic hiPSC, naïve and primed VP populations both displayed similar and equalized expressions of a broad array of vascular markers (e.g., CD31, CD34, CD144 and CD146) (FIG. 12C). Moreover, endothelial progenitor-specific (CD31⁺CD105^{hi}CD144⁺), pericytic (CD31⁺CD90⁺CD146⁺) populations³⁶ (FIG. 12C), and subcellular endothelial-specific organelles (e.g., Weibel-Palade (WP) bodies, and coated pits; FIG. 3D) were similarly expressed in both DVP and N-DVP; albeit with some minor differences (e.g., more abundant transcytotic endothelial channels (TEC) (FIG. 3D).

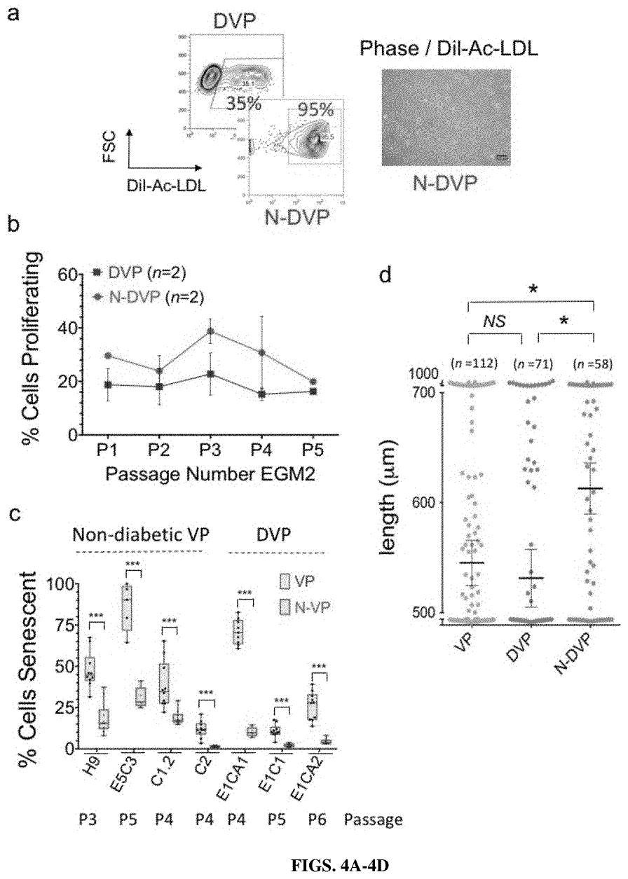

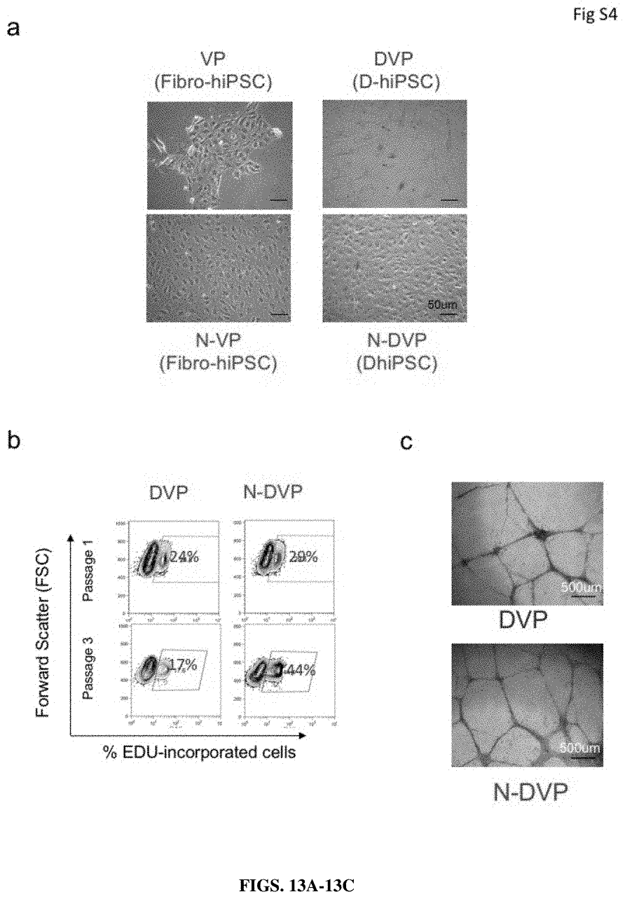

N-DVP possessed improved vascular functionality, lower culture senescence, and reduced sensitivity to DNA damage. Endothelium dysfunction in diabetics is characterized by poor EC survival, function, and DNA damage response (DDR). Although regenerative replacement of diseased vasculature requires high functioning cell therapies, previous studies of vascular differentiation with conventional fibroblast-hiPSC revealed poor and variable growth and expansion of vascular lineage cells, with high rates of apoptosis and early senescence^{34,35}. To evaluate endothelial functionality of naïve CD31⁺CD146⁺ VP, purified primed DVP vs N-DVP populations were re-cultured and expanded in endothelial growth medium (EGM2). N-DhiPSC-derived N-DVP were compared to isogenic primed DhiPSC-derived DVP for in vitro endothelial functionality with acetylated-Dil-LDL (Ac-Dil-Ac-LDL) uptake assays (FIG. 4A), 5-ethynyl-2-deoxyuridine (EdU) cell cycle proliferation assays, (FIGS. 4B, 13B), β-galactosidase senescence assays (FIGS. 4C, 13A), and quantitative Matrigel vascular tube formation (FIGS. 4D, 13C). These studies collectively revealed that CD31⁺CD146⁺-enriched N-DVP maintained higher endothelial Ac-Dil-Ac-LDL uptake function, more enhanced proliferation, and significantly less culture senescence than isogenic primed DVP counterparts in endothelial re-culture and expansion conditions.

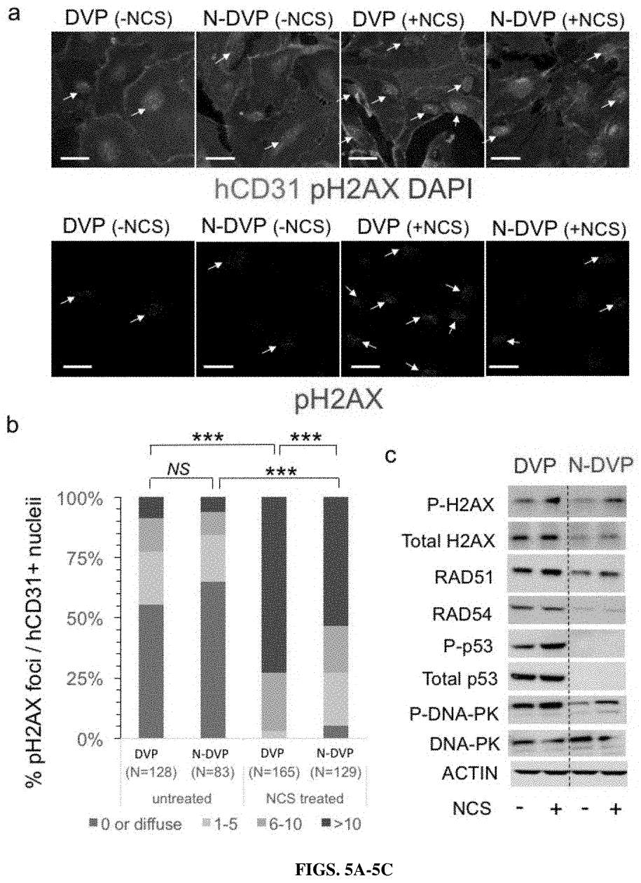

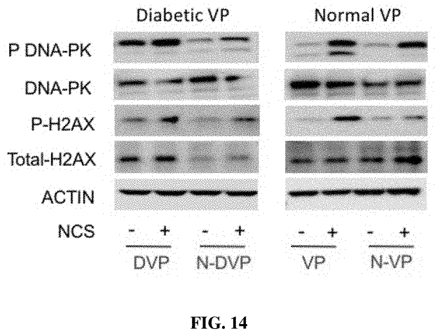

To further evaluate the relative resistance of N-DVP to senescence, genomic integrity maintenance was probed by assaying for sensitivity to double stranded DNA breaks (DSBs) following treatment with the radiation damage mimetic neocarzinostatin (NCS), which triggers both DDR and pH2AX-mediated reactive oxygen species (ROS) signals 37. Expression of phosphorylated p53 protein (P-p53), phosphorylated H2AX (pH2AX), RAD51, RAD54, phosphorylated DNA-PK (P-DNA-PK), which are all normally activated briefly following DDR and mediate repair of DSBs, were compared in re-cultured and expanded primed DVP vs N-DVP, before and after treatment with NCS (FIGS. 5A-5C). These studies revealed that levels of the DDR and DSB protein network (e.g., pH2AX, P-p53, RAD51, RAD54, and P-DNA-PK) were all globally reduced in their expressions in both non-diabetic N-VP and N-DVP (FIG. 14); both before and following NCS DNA damage exposure. Collectively, these data demonstrated that N-DVP may mediate a reduced sensitivity to stress-induced DNA damage than do primed DVP. These studies also suggested an improved genomic integrity of N-VP and N-DVP relative to VP generated from conventional counterparts of both normal hiPSC and diseased DhiPSC, respectively.

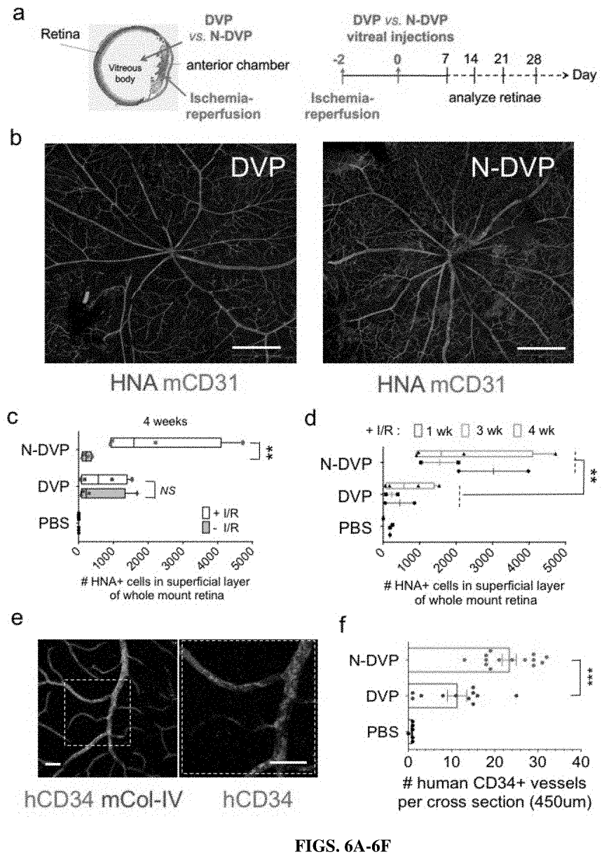

N-DVP injected into the vitreous of eyes survived, migrated into the neural retina, and engrafted into ischemia-damaged retinal vasculature with high efficiency. To evaluate the potential of N-DVP for in vivo engraftment and repair of ischemic retinal vessels, previously described humanized experimental NOG mouse model of ocular ischemia-reperfusion (I/R) injury was used, which allows the engraftment of human VP in an in vivo ischemic retinal niche (FIG. 6A). This model has been utilized extensively in rodents to recreate the ischemic damage observed in diabetic retinopathy². Intraocular pressure can be experimentally elevated to 90 mm Hg for 90 minutes in mice, followed by allowance of vascular reperfusion. This manipulation results in loss of retinal vasculature and apoptotic death of ischemic retinal neurons ˜7 days following I/R injury. Although this rodent model does not exactly simulate the sequence of events in diabetic retinopathy, it produces the same pathology seen in all ischemic retinal diseases: acellular capillaries and ischemic death of neurons. In the humanized immune-deficient mouse model, the sequential loss of murine host retinal vasculature ECs following ocular IR injury was detected with an antibody specific to mouse CD31 (mCD31), and the vascular basement membrane was detected with a murine-specific anti-collagen type-IV (mCol-IV) antibody. This approach is feasible because despite ischemic damage to acellular capillaries, the basement membrane shared by EC and pericytes in retinal capillaries remains intact. In this model, ischemic damage is more severe in capillaries and veins presumably due to their higher collapsible nature under increased intraocular pressure compared to arteries.

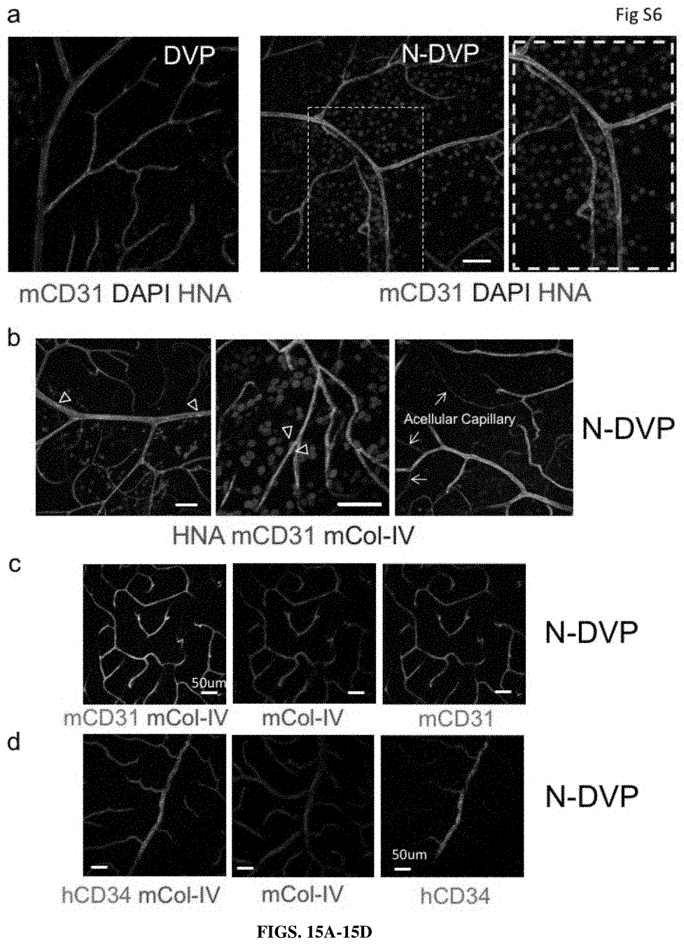

CD31⁺CD146⁺-sorted human DVP cells were differentiated from isogenic primed vs N-DhiPSC as above, cultured briefly in EGM2, and 50,000 primed DVP or N-DVP cells were injected in parallel directly into the vitreous body of NOG recipient eyes 2 days following IR injury (FIG. 6A). Human cell viability, intra-retinal migration, and engraftment in murine retina was evaluated at 1, 2, 3, and 4 weeks following human DVP injections with human-specific anti-human nuclear antigen (HNA) antibody staining. Analysis between 1-4 weeks following vitreous body injection in I/R-damaged eyes, HNA⁺ N-DVP were observed to survive in significantly greater frequencies than primed DVP within the superficial layer of the retina (FIGS. 6B-6D, FIG. 15A). Additionally, HNA⁺ N-DVP were observed to assume adherent abluminal pericytic and luminal endothelial positions with greater frequencies (FIG. 15B), and appeared to favor venous engraftment of blood vessels with large diameter than arteries suggesting a preferential migration in response to injury signals⁷. In contrast, and as previously demonstrated for non-diabetic fibroblast-derived hiPSC⁷, primed DVP cells examined at 2-4 weeks post-injection survived poorly and migrated inefficiently into ischemia-damaged blood vessels, and instead remained primarily in either the vitreous, or adherent to the adjacent superficial layer of the retina (FIGS. 6B-6D). N-DVP not only could be confirmed to significantly survive longer at 4 weeks post injection robustly in the superficial retinal ganglion cell layers (GCL) in response to injury (FIG. 6C), but also engrafted into murine retinal vessels with significantly greater human CD34⁺ grafting efficiencies than primed DVP (FIGS. 6E, 6F, 15C, 15D).

Interestingly, further analysis of deeper retinal vessels in transverse sections of the neural retina with anti-human CD34 and anti-human CD31 antibodies confirmed significantly higher endothelial engraftment from N-DVP than from primed DVP (FIGS. 7A-7D). At 2 weeks following I/R injury, human CD34⁺, and human CD31⁺ vessels were already readily detectable in N-DVP-injected retinae at significantly higher rates than primed DVP-injected retinae in the intima of murine host ischemia-damaged capillaries located in the ophthalmic artery distribution of the deep neural retinal layers. Notably, N-DVP efficiently migrated from the outer vascular layers of the GCL to form engrafted chimeric CD34⁺ and CD31⁺ human vessels in the deeper outer plexiform layers (OPL), inner nuclear layers (INL), and inner plexiform layers (IPL) of the murine neural retina (e.g., ˜5-20 CD34⁺ human-murine chimeric vessels per 450 μm cross section areas; FIGS. 7A-7D). In contrast, injected primed DVP-derived human CD34⁺ and CD31⁺ cells were detected primarily only in the superficial vascular layers of the retinal GCL or the inner limiting membrane (TLM); without further significant migration into deeper neural layers (FIGS. 7B, 7D). Collectively, these data demonstrated that N-DVP but not primed DVP migrated more efficiently from vitreous into the injured deep vascular neural retinal layers; suggesting that an efficient reparative injury-induced human-murine chimeric vasculogenesis had occurred following N-DVP (but not primed DVP) cellular therapy.

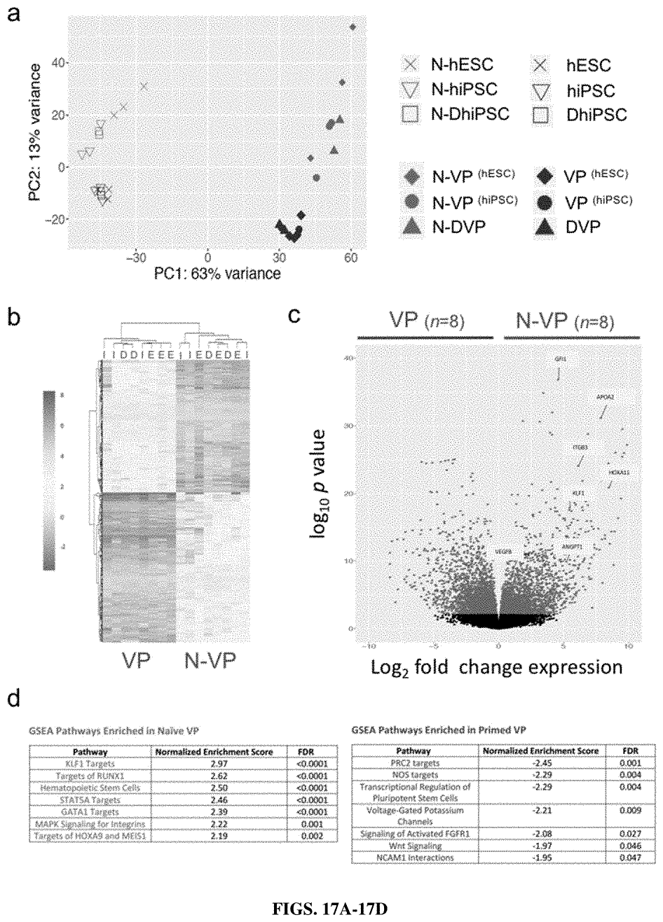

N-DhiPSC were configured with de-repressed, activation-poised bivalent histone marks at key developmental promoters, and tight regulation of ‘leaky’ lineage-primed gene expression. The murine naïve pluripotent state, which has higher differentiation potential than the primed murine pluripotent state¹¹, is distinguished by chromatin poised for unbiased gene activation³⁸ global reduction of CpG DNA methylation³⁹ and decreased repressive H3K27me3 histone deposition at bivalent Polycomb repressor Complex 2 (PRC2)-regulated promoter sites^{40,41}. To explore the molecular mechanisms that drive improved vascular functionality of LIF-3i-reverted N-DhiPSC, the global transcriptional profile and epigenetic configurations that may regulate a more faithful vascular gene expression in N-DVP were probed. The whole genome transcriptional profiles of naïve vs primed normal and diabetic VP, as well as their parental hPSC lines were evaluated by performing RNA-sequencing (RNA-Seq) FIGS. 17A-17D. Principal component analyses (PCAs) of VP vs N-VP transcriptomes demonstrated that both normal and diabetic samples possessed sharply distinguished global expression signatures (FIGS. 17A, 17B). A differential gene expression analysis revealed that naïve VP from both normal and diabetic sources were enriched in hemato-vascular stem-progenitor genes (e.g., ANGPT1, MMP9, VEGF, ITGB1, HOXA11, KLF1), and pathways (e.g., KLF1, RUNX1, STAT5A, GATA1 gene targets) (FIGS. 17C-17D).

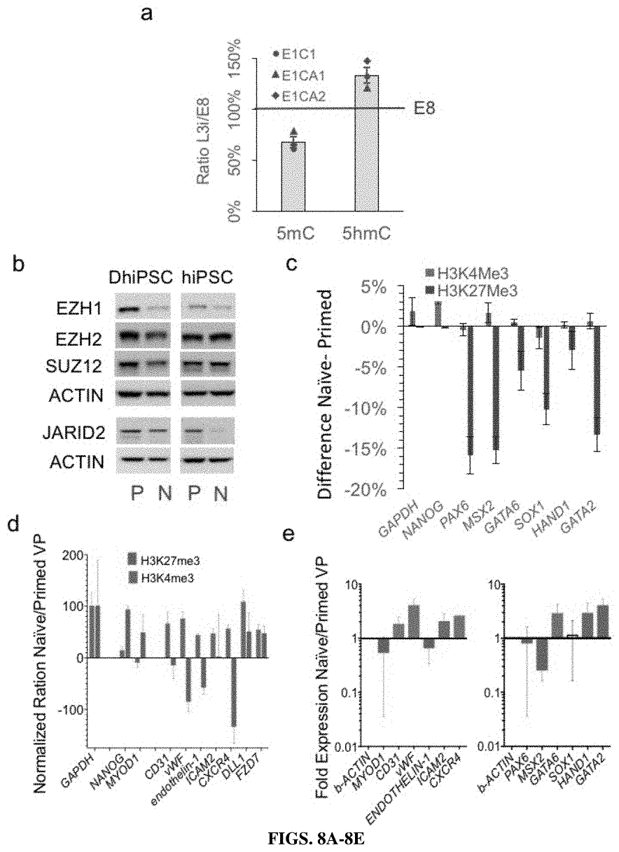

Additionally, all three fibroblast-N-DhiPSC lines exhibited significant reductions in global 5-methylcytosine (5MC)-associated CpG DNA methylation following LIF-3i reversion (FIG. 8A). The detection of increased global levels for 5-hydroxymethylcytosine (5hMC) in N-DhiPSC relative to primed DhiPSC further suggested a potential contribution of naive-like TET-mediated active CpG demethylation³⁹.

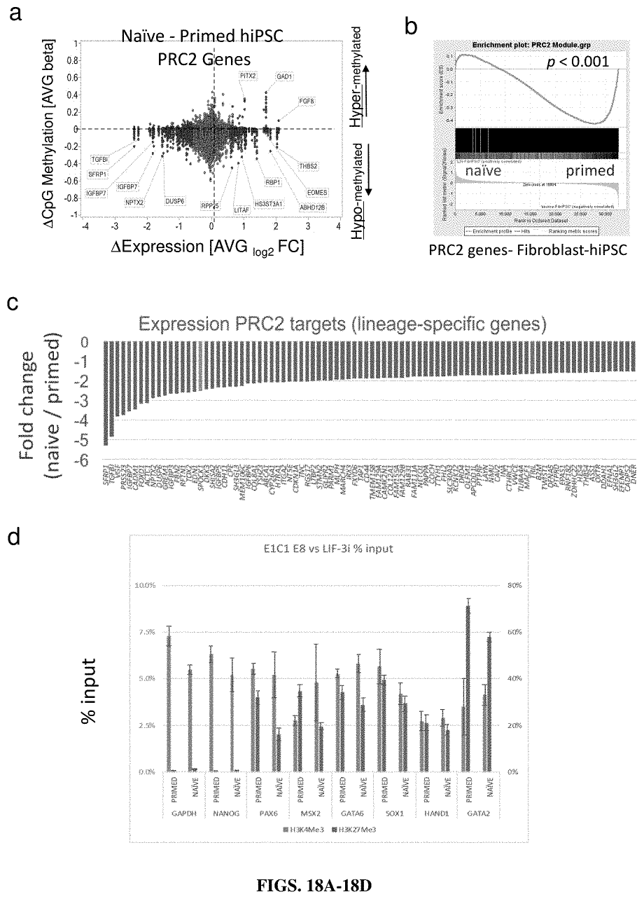

Gene-specific enrichment analysis (GSEA) of RNA-Seq VP samples revealed that primed VP were enriched in non-vascular-specific lineage genes (e.g. neuron-specific PRC2 gene targets) relative to N-VP, suggesting that lineage priming in conventional hPSC had affected not only the efficiency but also the epigenetic fidelity of vascular differentiation in primed VP (FIG. 17D). To better elucidate the epigenetic mechanisms regulating decreased lineage priming, a simultaneous bioinformatics analyses of both expression and CpG methylation of lineage-specifying PRC2 gene targets (before and after LIF-3i-reversion of a broad array of isogenic hiPSC lines), revealed that a broad rewiring of the lineage-specifying machinery. CpG DNA hypomethylation at promoter sites of PRC2 genes were broadly hypomethylated in N-hiPSC lines relative to their primed counterparts with a significant decrease of expression in many lineage-specifying PRC2 targets (FIGS. 18A, 18B). These studies revealed that in comparison to primed fibroblast-hiPSC, isogenic N-hiPSC displayed significantly less baseline epigenetic CpG methylation-regulated repression of lineage-specifying PRC2 genes, despite a broad silencing of lineage-primed transcriptional targets of PRC2 as previously reported¹² (FIG. 18C, Table 3, Table 4).

A critical mechanism for protecting naïve mouse ESC from lineage priming is via regulating the poised silencing or activation of lineage-specifying genes at bivalent H3K27me3 repressive and H3K4 activation histone marks, and RNA Polymerase II (POLII) pausing at promoter sites⁴⁰⁻⁴² Thus, the protein abundance of PRC2 components which mediate repressive H3K27me3 deposition on bivalent promoters in naïve versus primed normal and DhiPSCs was assessed (FIG. 8B). Interestingly, these studies revealed significantly decreased abundance of multiple components of the PRC2 complex in both diabetic and non-diabetic N-hiPSC, including the enzymatic subunits EZH1, EZH2, and the cofactor subunit JARID2⁴³ (which drives the localized recruitment of PRC2 at developmental promoters in mouse ESC). To functionally validate the activity of PRC2 targets, chromatin immunoprecipitation was employed, followed by qPCR (ChIP-PCR) on previously characterized lineage-specific bivalent gene promoters (e.g., PAX6, MSX2, GATA6, SOX1, HAND1, GATA2; (FIGS. 8C, 18D, Table 3, Table 4) to investigate the levels of bivalent active (H3K4me3) and repressive (H3K27me3) histone marks at these key lineage-specifying promoters. These studies revealed significant H3K27me3 reductions (5-15% from isogenic primed E1C1 and E1CA1 DhiPSC lines) following LIF-3i reversion.

Collectively, these CpG DNA methylation and histone mark studies revealed a relatively de-repressed naive epigenetic state in N-hiPSC that appeared more poised for activation than primed DhiPSC; with a potentially decreased barrier for multi-lineage gene activation relative to primed DhiPSC. Thus, as was previously demonstrated for naïve murine ESC^{38,40}, despite a tighter regulation of ‘leaky’ lineage-primed gene expression that was presumptively silenced through alternate naïve-like epigenetic mechanisms of bivalent promoter repression (e.g., promoter site RNA POLII pausing⁴⁰), N-hiPSC appeared poised with a lower epigenetic barrier for unbiased multi-lineage differentiation.

N-DVP possessed vascular lineage epigenetic de-repression and reduced non-vascular lineage-primed gene expression. To determine the downstream impact of a naïve epigenetic state with an apparently lower barrier for vascular lineage activation, the epigenetic configurations of vascular-lineage specific gene promoters in differentiated DVP and N-DVP by ChIP-PCR, were investigated. The promoters of downstream genes regulated by the PRC2-regulated factor GATA2 were selected, which promotes expression of genes of endothelial-specific identity and function (e.g., CD31, vWF, endothelin-1, and ICAM2)¹⁰. Promoters of genes known to be activated by chemical EZH2 and histone deacetylases (HDAC) de-repression in human endothelial progenitor cells (EPC) (e.g., CXCR4, DLL1, and FZD7) were selected⁴⁴. CD31⁺CD146⁺ DVPs vs N-DVP were MACS-purified, briefly expanded in EGM2, and ChIP-PCR was performed on promoter sites of these genes. Strikingly, relative to primed DVP, N-DVP displayed significantly increased marks for epigenetic activation (H3K4me3) and simultaneously reduced marks of promoter repression (H3K27me3) (FIG. 8D) for genes determining vascular functionality (e.g., CD31, vWF, endothelin-1, ICAM2, and CXCR4). Importantly, repressive H3K27me3 marks on N-DVP were increased relative to primed DVP for the non-vascular lineage muscle-specific promoter MYOD1. qRT-PCR expression analysis of these transcripts confirmed that naïve VP indeed expressed significantly higher levels of these vascular genes and lower levels of non-vascular genes (e.g., PAX6, MSX2, MYOD1) (FIG. 8E). These results were consistent with an improved epigenetic state in N-DhiPSC that potentiated a lower transcriptional barrier for generating N-DVP with higher vascular-specific gene expressions, decreased non-vascular lineage-primed gene expressions, and ultimately, presumptively greater in vivo functionality.

Discussion

To date, there has not been a human naïve pluripotent stem cell system demonstrating improved effectiveness over conventional hPSC for pre-clinical cellular therapies. These studies describe for the first time the advantage of employing an alternative tankyrase inhibitor-regulated human naïve pluripotent state for improving vascular regenerative therapies. Tankyrase inhibitor-regulated N-hiPSC represent a new class of human stem cells for regenerative medicine with improved multi-lineage functionality. In contrast, conventional hiPSC cultures adopt transcriptomic, epigenetic, and signaling signatures of lineage-primed pluripotency, and display a heterogeneous propensity for lineage bias and differentiation.

Herein, it was demonstrated that N-VP differentiated from both normal and diabetic patient-specific N-hiPSC maintained improved genomic stability, possessed higher expressions of vascular identity markers, and decreased expressions of non-vascular lineage-primed genes than VP generated from conventional, primed hiPSC. Moreover, N-DVP were functionally superior in migrating to and re-vascularizing the deep neural layers of the ischemic retina than DVP generated from conventional DhiPSC. Embryonic N-VP with prolific endothelial-pericytic potential and improved vascular functionality for re-vascularizing ischemia-damaged tissues can be generated in unlimited quantities and injected at multiple target sites for multiple treatments and time periods. Such epigenetically plastic N-VP are non-existent in circulating adult peripheral blood or bone marrow. For example, adult EPC are limited in multipotency, expansion, homing, and functionality in diabetes^{2,14-16}. The generation of embryonic N-DVP from a diabetic patient bypasses this obstacle. N-DhiPSC are more effectively reprogrammed from a donor's skin or blood cells back to a pre-diseased state, and could subsequently be differentiated to unlimited quantities of pristine, transplantable N-DVP; which unlike adult diabetic EPC would be unaffected by the functional and epigenetic damage caused by chronic hyperglycemia.

Previous studies demonstrated that embryonic VP derived from conventional CB-derived hiPSC generated with higher and more complete reprogramming efficiencies had decreased lineage-primed gene expression and displayed limited but long-term regeneration of degenerated retinal vessels⁷. In comparison, conventional skin fibroblast-derived hiPSC lines with higher rates of reprogramming errors and lineage-primed gene expression displayed poorer vascular differentiation and in vivo retinal engraftment efficiencies relative to conventional (non-isogenic) CB-hiPSC. Here, this obstacle was solved for diabetic skin fibroblast donor-derived hiPSC by demonstrating that CD31⁺CD146⁺ endothelial-pericytic N-DVP were more efficiently generated from N-DhiPSC than from conventional DhiPSC. Additionally, N-DVP had higher epigenomic stability, reduced lineage priming, and improved in vivo engraftment capacity in ischemia-damaged blood vessels. In future clinical studies, multiple cell types (e.g., vascular endothelium, pericytes, retinal neurons, glia, and retinal pigmented epithelium) could all potentially be differentiated from the same autologous or HLA-compatible, banked patient-specific hiPSC line for a comprehensive repair of ischemic vascular and macular degenerative disease.

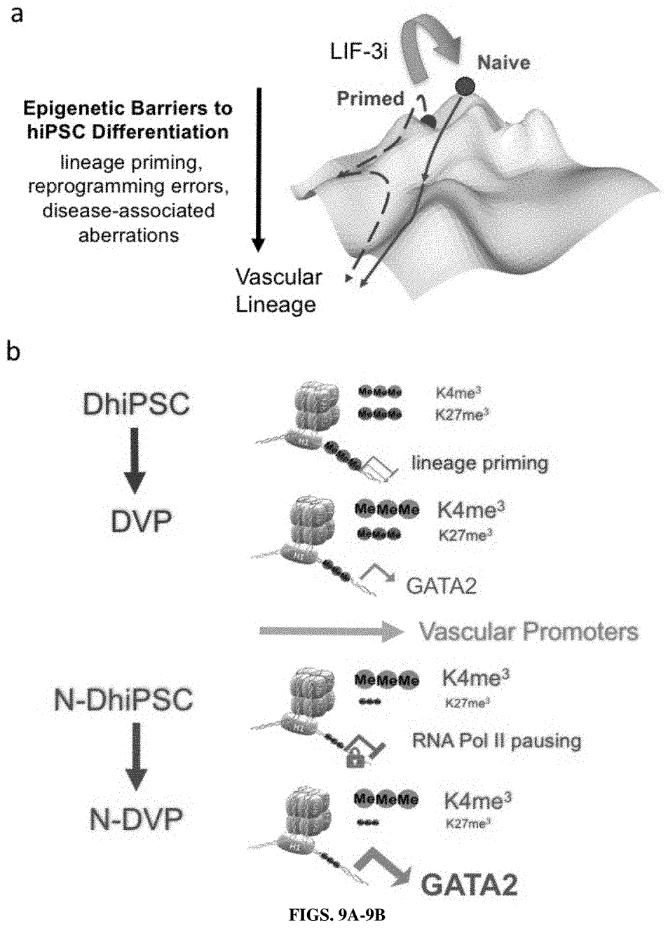

The studies herein have also demonstrated that the obstacles of incomplete reprogramming, lineage priming, and disease-associated epigenetic aberrations in conventional hiPSC can be overcome with molecular reversion to a tankyrase inhibitor-regulated naïve epiblast-like state with a more primitive, unbiased epigenetic configuration. N-DhiPSC possessed a naïve epiblast-like state with decreased epigenetic barriers for vascular lineage specification, and decreased non-vascular lineage specific gene expression (FIG. 9A). Interestingly, compared to conventional lineage primed DhiPSC, tankyrase-inhibited N-DhiPSC possessed a de-repressed naïve epiblast-like epigenetic configuration at bivalent developmental promoters that was highly poised for non-biased, multi-lineage lineage specification, and was configured in a manner akin to naïve murine ESC³⁸⁻⁴¹ (FIG. 9B).

The mechanism by which the tankyrase/PARP inhibitor XAV939 stabilized and expanded the functional pluripotency of an inherently unstable human naïve state in classical 2i conditions currently remains incompletely defined. However, without wishing to be bound by theory, it was hypothesized that a potential epigenetic mechanism is that CpG DNA methylation and histone configurations at developmental promoters of diabetic N-hiPSC possessed tight regulation of lineage-specific gene expression and a de-repressed naïve epiblast-like epigenetic state that was highly poised for multi-lineage transcriptional activation. Furthermore, the LIF-3i chemical cocktail minimally employs MEK inhibition (PD0325901) to block lineage-primed differentiation, along with a simultaneous and parallel dual synergy of XAV939 with the GSK30 (CHIR99021) inhibitor to augment WNT signalling²⁰. The presumptive mechanism of augmented WNT signalling is via inhibition of tankyrase-mediated degradation of AXIN, which causes stabilization and increased cytoplasmic retention of the activated isoform of β-catenin in murine ESC (which decreases β-catenin-TCF interactions). However, in humans, the repertoire of proteins directly targeted by tankyrase post-translational PARylation extends far beyond WNT signalling, and includes proteins (e.g., AXIN1 and 2, APC2, NKD1, NKD2, and HectD1) with diverse biological functions that potentially cooperate to support a stable pluripotent state 47. These functions include regulation of telomere elongation and cohesion (TRF1), YAP signalling (angiomotin), mitotic spindle integrity (NuMa), GLUT4 vesicle trafficking (IRAP), DNA damage response regulation (CHEK2), and microRNA processing (DICER). Interestingly, TRF1 was identified as an essential factor for iPSC reprogramming in mouse and human PSC⁴⁸. Additionally, although LIF-3i includes MEK inhibition and promotes global and genome-wide low DNA methylation, it does not appear to impair genomic CpG methylation at imprinted loci¹². Although the mechanism of such imprint preservation by XAV939 in the context of MEK inhibitor is currently obscure, PARylation has been shown to safeguard the Dnmt1 promoter in mouse cells, and antagonizes aberrant hypomethylation at CpG islands, including at imprinted genes^{49,51}. Thus, the role of PARylation on DNA methylation requires deeper investigation.

Diabetic hyperglycemic alterations of blood vessel viability and integrity lead to multi-organ dysfunction that results in endothelial dysfunction linked to epigenetic remodeling¹⁷ (e.g., DNA methylation⁵¹, histone marks^{52,53} and oxidative stress^{54,55}). Several studies have shown that these aberrant epigenetic changes may be partially overcome by genome-wide chemical treatments that restore some endothelial function^{56,57}. The extent of retention of diseased ‘diabetic epigenetic memory’ at developmental genes from incomplete or ineffective reprogramming within DhiPSC-derived lineages and its role in impaired regenerative capacity remains unclear, and marked by high variability in differentiation efficiency or retention of diseased phenotype 58-61 For example, endothelial differentiation of iPSC generated from diabetic mice displayed vascular dysfunction, impaired in vivo regenerative capacity, and diabetic iPSC displayed poor teratoma formation⁶³. Human iPSC from patients with rare forms of diabetes-related metabolic disorders have similarly shown significant functional endothelial impairment⁵⁸. Transient chemical demethylation of T1D-hiPSC was sufficient to restore differentiation in resistant cell lines and achieve functional differentiation into insulin-producing cells¹⁸.

In summary, these studies have demonstrated that highly functional N-VP cells can be generated independent of genetic background or diseased origin from a diseased N-hPSC. Naïve reversion of conventional DhiPSC may potentiate an epigenetic remodeling of reprogrammed diabetic fibroblasts that avoided differentiation into dysregulated in dysfunctional ECs with ‘diabetic epigenetic memory’. Similarly, tankyrase inhibitor-regulated N-DhiPSC are expected to improve the poor and variable DhiPSC differentiation generation of other affected tissues in diabetes⁶⁴ including pancreatic, renal, hematopoietic, retinal, and cardiac lineages. It is proposed herein, that autologous or cell-banked transplantable progenitors derived from tankyrase inhibitor-regulated N-hiPSC will more effectively reverse the epigenetic pathology that drive diseases such as diabetes. The application of this new class of human stem cells may inspire further new directions of investigation for understanding human pluripotency, and for improving the utility of hiPSC therapies in regenerative medicine. The further optimization of tankyrase-inhibited human naïve pluripotent stem cells in defined, clinical-grade conditions may significantly advance regenerative medicine.

### REFERENCES

1 Lutty, G. A. Diabetic choroidopathy. Vision Res 139, 161-167, doi:10.1016/j.visres.2017.04.011 (2017).
2 Zheng, L., Gong, B., Hatala, D. A. & Kern, T. S. Retinal ischemia and reperfusion causes capillary degeneration: similarities to diabetes. Invest Ophthalmol Vis Sci 48, 361-367, doi:10.1167/iovs.06-0510 (2007).
3 Joussen, A. M. et al. A central role for inflammation in the pathogenesis of diabetic retinopathy. FASEB J 18, 1450-1452, doi:10.1096/fj.03-1476fje (2004).
4 Joussen, A. M. et al. Leukocyte-mediated endothelial cell injury and death in the diabetic retina. Am J Pathol 158, 147-152, doi:10.1016/50002-9440(10)63952-1 (2001).
5 D'Amore, P. A. Mechanisms of retinal and choroidal neovascularization. Invest Ophthalmol Vis Sci 35, 3974-3979 (1994).
6 Glaser, B. M., D'Amore, P. A., Michels, R. G., Patz, A. & Fenselau, A. Demonstration of vasoproliferative activity from mammalian retina. J Cell Biol 84, 298-304, doi:10.1083/jcb.84.2.298 (1980).
7 Park, T. S. et al. Vascular progenitors from cord blood-derived induced pluripotent stem cells possess augmented capacity for regenerating ischemic retinal vasculature. Circulation 129, 359-372, doi:10.1161/CIRCULATIONAHA.113.003000 (2014).
8 Dar, A. et al. Multipotent vasculogenic pericytes from human pluripotent stem cells promote recovery of murine ischemic limb. Circulation 125, 87-99, doi:10.1161/CIRCULATIONAHA.111.048264 (2012).
9 Mandai, M., Kurimoto, Y. & Takahashi, M. Autologous Induced Stem-Cell-Derived Retinal Cells for Macular Degeneration. N Engl J Med 377, 792-793, doi:10.1056/NEJMc1706274 (2017).
10 Sharma, R. et al. Clinical-grade stem cell-derived retinal pigment epithelium patch rescues retinal degeneration in rodents and pigs. Sci Transl Med 11, doi:10.1126/scitranslmed.aat5580 (2019).
11 Zimmerlin, L., Park, T. S. & Zambidis, E. T. Capturing Human Naive Pluripotency in the Embryo and in the Dish. Stem Cells Dev 26, 1141-1161, doi:10.1089/scd.2017.0055 (2017).
12 Zimmerlin, L. et al. Tankyrase inhibition promotes a stable human naive pluripotent state with improved functionality. Development 143, 4368-4380, doi:10.1242/dev.138982 (2016).
13 Park, T. S., Zimmerlin, L., Evans-Moses, R. & Zambidis, E. T. Chemical Reversion of Conventional Human Pluripotent Stem Cells to a Naive-like State with Improved Multilineage Differentiation Potency. J Vis Exp, doi:10.3791/57921 (2018).
14 Brunner, S. et al. Circulating angiopoietic cells and diabetic retinopathy in type 2 diabetes mellitus, with or without macrovascular disease. Invest Ophthalmol Vis Sci 52, 4655-4662, doi:10.1167/iovs.10-6520 (2011).
15 Caballero, S. et al. Ischemic vascular damage can be repaired by healthy, but not diabetic, endothelial progenitor cells. Diabetes 56, 960-967, doi:10.2337/db06-1254 (2007).
16 Jarajapu, Y. P. & Grant, M. B. The promise of cell-based therapies for diabetic complications: challenges and solutions. Circ Res 106, 854-869, doi:10.1161/CIRCRESAHA.109.213140 (2010).
17 Khullar, M., Cheema, B. S. & Raut, S. K. Emerging Evidence of Epigenetic Modifications in Vascular Complication of Diabetes. Front Endocrinol (Lausanne) 8, 237, doi:10.3389/fendo.2017.00237 (2017).
18 Manzar, G. S., Kim, E. M. & Zavazava, N. Demethylation of induced pluripotent stem cells from type 1 diabetic patients enhances differentiation into functional pancreatic beta cells. J Biol Chem 292, 14066-14079, doi:10.1074/jbc.M117.784280 (2017).
19 Yang, J. et al. Establishment of mouse expanded potential stem cells. Nature 550, 393-397, doi:10.1038/nature24052 (2017).
20 Kim, H. et al. Modulation of beta-catenin function maintains mouse epiblast stem cell and human embryonic stem cell self-renewal. Nat Commun 4, 2403, doi:10.1038/ncomms3403 (2013).
21 Gao, X. et al. Establishment of porcine and human expanded potential stem cells. Nat Cell Biol 21, 687-699, doi:10.1038/s41556-019-0333-2 (2019).
22. Theunissen, T. W., et al., Molecular Criteria for Defining the Naive Human Pluripotent State. Cell Stem Cell, 2016.
23. Pastor, W. A., et al., Naive Human Pluripotent Cells Feature a Methylation Landscape Devoid of Blastocyst or Germline Memory. Cell Stem Cell, 2016. 18(3): p. 323-9.
24. Choi, J., et al., Prolonged Mek1/2 suppression impairs the developmental potential of embryonic stem cells. Nature, 2017. 548(7666): p. 219-223.
25. Warrier, S., et al., Direct comparison of distinct naive pluripotent states in human embryonic stem cells. Nat Commun, 2017. 8: p. 15055.
26. Lee, J. H., et al., Lineage-Specific Differentiation Is Influenced by State of Human Pluripotency. Cell Rep, 2017. 19(1): p. 20-35.
27 Park, T. S. et al. Growth factor-activated stem cell circuits and stromal signals cooperatively accelerate non-integrated iPSC reprogramming of human myeloid progenitors. PLoS ONE7, e42838, doi:10.1371/journal.pone.0042838 (2012).
28 Burridge, P. W. et al. A universal system for highly efficient cardiac differentiation of human induced pluripotent stem cells that eliminates interline variability. PLoS ONE 6, e18293, doi:10.1371/journal.pone.0018293 (2011).
29 Bar-Nur, O. et al. Small molecules facilitate rapid and synchronous iPSC generation. Nat Methods 11, 1170-1176, doi:10.1038/nmeth.3142 (2014).
30. Bai, P. Biology of poly(ADP-ribose) polymerases: the factotums of cell maintenance. (2015), Mol Cell, 58, 947-958, http://dx.doi.org/10.1016/j.molcel.2015.01.034
31. Pastor, W. A., et al., TFAP2C regulates transcription in human naive pluripotency by opening enhancers. Nat Cell Biol, 2018. 20(5): p. 553-564.
32. Ng, E. S., Davis, R., Stanley, E. G. & Elefanty, A. G. A protocol describing the use of a recombinant protein-based, animal product-free medium (APEL) for human embryonic stem cell differentiation as spin embryoid bodies. Nature protocols 3, 768-776, doi:10.1038/nprot.2008.42 (2008).
33. Orlova, V. V. et al. Generation, expansion and functional analysis of endothelial cells and pericytes derived from human pluripotent stem cells. Nat Protoc 9, 1514-1531, doi:10.1038/nprot.2014.102 (20014).
34. Feng, Q. et al. Hemangioblastic derivatives from human induced pluripotent stem cells exhibit limited expansion and early senescence. Stem Cells 28, 704-712, doi:10.1002/stem.321 (2009).
35. Rufaihah, A. J. et al. Human induced pluripotent stem cell-derived endothelial cells exhibit functional heterogeneity. American journal of translational research 5, 21-35 (2013).
36. Crisan, M. A perivascular origin for mesenchymal stem cells in multiple human organs. Cell Stem Cell (2008).
37. Kuo, W. L., Meyn, R. E. & Haidle, C. W. Neocarzinostatin-mediated DNA damage and repair in wild-type and repair-deficient Chinese hamster ovary cells. Cancer Res 44, 1748-1751 (1984).
38. Marks, H. et al. The transcriptional and epigenomic foundations of ground state pluripotency. Cell 149, 590-604, doi:10.1016/j.cell.2012.03.026 (2012).
39 Leitch, H. G. et al. Naive pluripotency is associated with global DNA hypomethylation. Nat Struct Mol Biol 20, 311-316, doi:10.1038/nsmb.2510 (2013).
40. Tee, W. W., Shen, S. S., Oksuz, O., Narendra, V. & Reinberg, D. Erk1/2 activity promotes chromatin features and RNAPII phosphorylation at developmental promoters in mouse ESCs. Cell 156, 678-690, doi:10.1016/j.cell.2014.01.009 (2014).
41. Thornton, S. R., Butty, V. L., Levine, S. S. & Boyer, L. A. Polycomb Repressive Complex 2 regulates lineage fidelity during embryonic stem cell differentiation. PLoS One 9, e110498, doi:10.1371/journal.pone.0110498 (2014).
42. Shan, Y. et al. PRC2 specifies ectoderm lineages and maintains pluripotency in primed but not naive ESCs. Nat Commun 8, 672, doi:10.1038/s41467-017-00668-4 (2017).
43. Landeira, D. et al. Jarid2 is a PRC2 component in embryonic stem cells required for multi-lineage differentiation and recruitment of PRC1 and RNA Polymerase II to developmental regulators. Nat Cell Biol 12, 618-624, doi:10.1038/ncb2065 (2010).
44. Fraineau, S. et al. Epigenetic Activation of Pro-angiogenic Signaling Pathways in Human Endothelial Progenitors Increases Vasculogenesis. Stem Cell Reports 9, 1573-1587, doi:10.1016/j.stemcr.2017.09.009 (2017).
45. Yang, Y. et al. Derivation of Pluripotent Stem Cells with In Vivo Embryonic and Extraembryonic Potency. Cell 169, 243-257 e225, doi:10.1016/j.cell.2017.02.005 (2017).
46. Guo, G. et al. Epigenetic resetting of human pluripotency. Development 144, 2748-2763, doi:10.1242/dev.146811 (2017).
47. Bhardwaj, A., Yang, Y., Ueberheide, B. & Smith, S. Whole proteome analysis of human tankyrase knockout cells reveals targets of tankyrase-mediated degradation. Nature communications 8, 2214, doi:10.1038/s41467-017-02363-w (2017).
48. Schneider, R. P. et al. TRF1 is a stem cell marker and is essential for the generation of induced pluripotent stem cells. Nature communications 4, 1946, doi:10.1038/ncomms2946 (2013).
49. De Vos, M. et al. Poly(ADP-ribose) polymerase 1 (PARP1) associates with E3 ubiquitin-protein ligase UHRF1 and modulates UHRF1 biological functions. J Biol Chem 289, 16223-16238, doi:10.1074/jbc.M113.527424 (2014).
50. Zampieri, M. et al. Parp1 localizes within the Dnmt1 promoter and protects its unmethylated state by its enzymatic activity. PLoS One 4, e4717, doi:10.1371/journal.pone.0004717 (2009).
51. Vigorelli, V. et al. Abnormal DNA Methylation Induced by Hyperglycemia Reduces CXCR 4 Gene Expression in CD 34(+) Stem Cells. J Am Heart Assoc 8, e010012, doi:10.1161/JAHA.118.010012 (2019).
52. Liao, Y. et al. NADPH oxidase 4 and endothelial nitric oxide synthase contribute to endothelial dysfunction mediated by histone methylations in metabolic memory. Free Radic Biol Med 115, 383-394, doi:10.1016/j.freeradbiomed.2017.12.017 (2018).
53. Hou, Q., Hu, K., Liu, X., Quan, J. & Liu, Z. HADC regulates the diabetic vascular endothelial dysfunction by targetting MnSOD. Biosci Rep 38, doi:10.1042/BSR20181042 (2018).
54 Incalza, M. A. et al. Oxidative stress and reactive oxygen species in endothelial dysfunction associated with cardiovascular and metabolic diseases. Vascul Pharmacol 100, 1-19, doi:10.1016/j.vph.2017.05.005 2018).
55 Hammes, H. P. Diabetic retinopathy: hyperglycaemia, oxidative stress and beyond. Diabetologia 61, 29-38, doi:10.1007/s00125-017-4435-8 (2018).
56 Wu, J. et al. Sodium butyrate attenuates diabetes-induced aortic endothelial dysfunction via P300-mediated transcriptional activation of Nrf2. Free Radic Biol Med 124, 454-465, doi:10.1016/j.freeradbiomed.2018.06.034 (2018).
57 Xie, M. Y., Yang, Y., Liu, P., Luo, Y. & Tang, S. B. 5-aza-2′-deoxycytidine in the regulation of antioxidant enzymes in retinal endothelial cells and rat diabetic retina. Int J Ophthalmol 12, 1-7, doi:10.18240/ijo.2019.01.01 (2019).
58. Carcamo-Orive, I., Huang, N. F., Quertermous, T. & Knowles, J. W. Induced Pluripotent Stem Cell-Derived Endothelial Cells in Insulin Resistance and Metabolic Syndrome. Arterioscler Thromb Vasc Biol 37, 2038-2042, doi:10.1161/ATVBAHA.117.309291 (2017).
59. Chan, X. Y. et al. Three-Dimensional Vascular Network Assembly From Diabetic Patient-Derived Induced Pluripotent Stem Cells. Arterioscler Thromb Vasc Biol 35, 2677-2685, doi:10.1161/ATVBAHA.115.306362 (2015).
60. Drawnel, F. M. et al. Disease modeling and phenotypic drug screening for diabetic cardiomyopathy using human induced pluripotent stem cells. Cell Rep 9, 810-821, doi:10.1016/j.celrep.2014.09.055 (2014).
61. Gu, M. et al. Pravastatin reverses obesity-induced dysfunction of induced pluripotent stem cell-derived endothelial cells via a nitric oxide-dependent mechanism. Eur Heart J 36, 806-816, doi:10.1093/eurheartj/ehu411 (2015).
62. Kikuchi, C. et al. Comparison of Cardiomyocyte Differentiation Potential Between Type 1 Diabetic Donor- and Nondiabetic Donor-Derived Induced Pluripotent Stem Cells. Cell Transplant 24, 2491-2504, doi:10.3727/096368914X685762 (2015).
63. Stepniewski, J. et al. Induced pluripotent stem cells as a model for diabetes investigation. Sci Rep 5, 8597, doi:10.1038/srep08597 (2015).
64. Thatava, T. et al. Intrapatient variations in type 1 diabetes-specific iPS cell differentiation into insulin-producing cells. Mol Ther 21, 228-239, doi:10.1038/mt.2012.245 (2013).
65. Kanki, Y. et al. Epigenetically coordinated GATA2 binding is necessary for endothelium-specific endomucin expression. EMBOJ 30, 2582-2595, doi:10.1038/emboj.2011.173 (2011).
66. Subramanian, Tamayo, et al. (2005, Proc. Natl. Acad. Scie. USA, 102, 15545-15550) and Mootha, Lindgren, et al. (2003), Nat Genet 34, 267-273.
67. Dobin A, Davis C A, Schlesinger F, Drenkow J, Zaleski C, Jha S, et al. STAR: ultrafast universal RNA-seq aligner. Bioinformatics. 2013; 29(1):15-21.
68. Love M I, Huber W, Anders S. Moderated estimation of fold change and dispersion for RNA-seq data with DESeq2. Genome Biol. 2014; 15(12):550.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

contacting a human induced pluripotent stem cell (hiPSC) with a composition comprising a leukemia inhibitory factor (LIF) and at least one or more agents which inhibit one or more signaling pathways to produce a naïve human induced pluripotent stem cell (N-hiPSC);

administering to the subject, a composition comprising an effective amount of the naïve human induced pluripotent stem cells (N-hiPSC), wherein the N-hiPSC differentiate and revascularize the subject's vascular system, thereby treating the vascular disorder.

1. A method of treating an ischemic retina of a subject in need thereof, comprising:
administering to the subject, a composition comprising an effective amount of the naïve human induced pluripotent stem cells (N-hiPSC), wherein the N-hiPSC differentiate and revascularize the subject's ischemic retina, thereby treating the ischemic retina,
wherein the N-hiPSC are obtainable by steps comprising contacting a human induced pluripotent stem cell (hiPSC) with a composition comprising a leukemia inhibitory factor (LIF) and at least one or more agents which inhibit one or more signaling pathways to produce the N-hiPSC.

2. A method of treating an ischemic retina of a subject in need thereof, comprising:
contacting a human induced pluripotent stem cell (hiPSC) with a composition comprising a leukemia inhibitory factor (LIF) and at least one or more agents which inhibit one or more signaling pathways to produce a naïve human induced pluripotent stem cell (N-hiPSC);
administering to the subject, a composition comprising an effective amount of the naïve human induced pluripotent stem cells (N-hiPSC), wherein the N-hiPSC differentiate and revascularize the subject's ischemic retina, thereby
treating the ischemic retina.

3. The method of claim 1, wherein the one or more agents comprise simultaneous uses of inhibitors of tankyrase, mitogen-activated protein kinase kinase (MEK), Glycogen Synthase Kinase 3-β (GSK3β) or signaling pathways thereof.

4. The method of claim 3, wherein a tankyrase inhibitor comprises: XAV939, IWR-1, G007-LK, JW55, AZ1366, JW 74, NVP-TNKS656 or combinations thereof.

5. The method of claim 3, wherein a GSK3β inhibitor comprises: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR 99021), 5-Ethyl-7,8-dimethoxy-1H-pyrrolo[3,4-c]isoquinoline-1,3(2H)-dione (3F8), 1-(7-Methoxyquinolin-4-yl)-3-[6-(trifluoromethyl)pyridin-2-yl]urea (A 1070722), N⁶-[2-[[4-(2,4-Dichlorophenyl)-5-(1H-imidazol-1-yl)-2-pyrimidinyl]amino]ethyl]-3-nitro-2,6-pyridinediamine (CHIR 98014), lithium chloride (LiCl), 4-benzyl-2-methyl-1,2,4-thiadiazolidine-3,5-dione (TDZD-8), 5-iodo-indirubin-3′-monoxime (I3′M) and N-(4-methoxybenzyl)-N′-(5-nitro-1,3-thiazol-2-yl)urea (AR-A014418) or combinations thereof.

6. The method of claim 3, wherein MEK inhibitor comprises: PD032590, CI-1040 (PD184352), cobimetinib (GDC-0973, XL518), Selumetinib (AZD6244), MEK162, AZD8330, TAK-733, GDC-0623, Refametinib (RDEA119; BAY 869766), Pimasertib (AS703026), RO4987655 (CH4987655), RO5126766, WX-554, HL-085 or combinations thereof.

7. The method of claim 1, wherein the hiPSCs are derived from primed isogenic hiPSCs.

8. The method of claim 1, wherein the hiPSC are derived from diabetic donor hiPSCs (DhiPSC) or non-diabetic donor hiPSCs.

9. The method of claim 1 wherein the N-hiPSC are obtained by steps comprising contacting a human induced pluripotent stem cell (hiPSC) with a composition comprising a leukemia inhibitory factor (LIF) and at least one or more agents which inhibit one or more signaling pathways to produce the N-hiPSC

10. A method of producing a vascular progenitor (VP) cell comprising:
contacting a human induced pluripotent stem cell (hiPSC) with a composition comprising a leukemia inhibitory factor (LIF) and at least one agent or a simultaneous combination of at least three agents which inhibit one or more signaling pathways to produce a naïve human induced pluripotent stem cell (N-hiPSC); and,
differentiating the N-hiPSC in vitro or by implantation in vivo.

11. The method of claim 10, wherein the at least one agent is an inhibitor of poly-ADP-ribosyltransferase and signaling pathways thereof.

12. The method of claim 10, wherein the at least one agent is an inhibitor of mitogen-activated protein kinase kinase (MEK) and signaling pathways thereof.

13. The method of claim 10, wherein the at least one agent is an inhibitor of Glycogen Synthase Kinase 3 (GSK3) or signaling pathways thereof.

14. The method of claim 10, wherein the composition comprising a combination of at least three agents comprises inhibitors of poly-ADP-ribosyltransferase, MEK, GSK3 and signaling pathways thereof.

15. The method of claim 10, wherein the poly-ADP-ribosyltransferase is tankyrase.

16. The method of claim 10, wherein the GSK3 is a GSK3β isoform.

17. The method of claim 15, wherein a tankyrase inhibitor comprises: XAV939, IWR-1, G007-LK, JW55, AZ1366, JW 74, NVP-TNKS656 or combinations thereof.

18-25. (canceled)

26. A composition comprising an effective amount of naïve human induced pluripotent stem cells (N-hiPSCs) wherein the N-hiPSCs are tankyrase inhibitor regulated.

27. The composition of claim 26, wherein the hiPSC is reprogrammed from donor diabetic or donor non-diabetic fibroblasts.

28-29. (canceled)


SEQUENCE LISTING (32 sequences)

SEQ ID NO: 1 - DNA, 20 bp, Artificial Sequence
  Description of Artificial Sequence Synthetic primer
tgcaaagagcaaaggtcaaa

SEQ ID NO: 2 - DNA, 20 bp, Artificial Sequence
  Description of Artificial Sequence Synthetic primer
cacgcctagccaaaatcact

SEQ ID NO: 3 - DNA, 20 bp, Artificial Sequence
  Description of Artificial Sequence Synthetic primer
cagtagagacactgaggccc

SEQ ID NO: 4 - DNA, 20 bp, Artificial Sequence
  Description of Artificial Sequence Synthetic primer
ttggaagcttggccctactt

SEQ ID NO: 5 - DNA, 20 bp, Artificial Sequence
  Description of Artificial Sequence Synthetic primer
tgtctggggctggaataaag

SEQ ID NO: 6 - DNA, 20 bp, Artificial Sequence
  Description of Artificial Sequence Synthetic primer
gacttggacagctctctgcc

SEQ ID NO: 7 - DNA, 20 bp, Artificial Sequence
  Description of Artificial Sequence Synthetic primer
gcatggctaatgagatgcaa

SEQ ID NO: 8 - DNA, 20 bp, Artificial Sequence
  Description of Artificial Sequence Synthetic primer
cattgagaggagggtttgga

SEQ ID NO: 9 - DNA, 20 bp, Artificial Sequence
  Description of Artificial Sequence Synthetic primer
gccaatcagaaaacgctacc

SEQ ID NO: 10 - DNA, 20 bp, Artificial Sequence
  Description of Artificial Sequence Synthetic primer
gcaaccatttgatcctccat

SEQ ID NO: 11 - DNA, 20 bp, Artificial Sequence
  Description of Artificial Sequence Synthetic primer
cggctactagcggttttacg

SEQ ID NO: 12 - DNA, 20 bp, Artificial Sequence
  Description of Artificial Sequence Synthetic primer
aagaagatgcggctgactgt

SEQ ID NO: 13 - DNA, 20 bp, Artificial Sequence
  Description of Artificial Sequence Synthetic primer
ctgtgcccaggtaaccaaat

SEQ ID NO: 14 - DNA, 20 bp, Artificial Sequence
  Description of Artificial Sequence Synthetic primer
ctcgctcggtgagtttcttc

SEQ ID NO: 15 - DNA, 20 bp, Artificial Sequence
  Description of Artificial Sequence Synthetic primer
ggatgagaacggtttctgga

SEQ ID NO: 16 - DNA, 20 bp, Artificial Sequence
  Description of Artificial Sequence Synthetic primer
ttgtgaacttgtggctcctg

SEQ ID NO: 17 - DNA, 20 bp, Artificial Sequence
  Description of Artificial Sequence Synthetic primer
ggcaaggctgaaaatgagac

SEQ ID NO: 18 - DNA, 20 bp, Artificial Sequence
  Description of Artificial Sequence Synthetic primer
gatagccactcccccttttc

SEQ ID NO: 19 - DNA, 19 bp, Artificial Sequence
  Description of Artificial Sequence Synthetic primer
ctgcctggagggagatggt

SEQ ID NO: 20 - DNA, 27 bp, Artificial Sequence
  Description of Artificial Sequence Synthetic primer
aagatctcagataatgagaggaaatgc

SEQ ID NO: 21 - DNA, 20 bp, Artificial Sequence
  Description of Artificial Sequence Synthetic primer
aggcctctaatcctcgaagc

SEQ ID NO: 22 - DNA, 20 bp, Artificial Sequence
  Description of Artificial Sequence Synthetic primer
ccctctcgtttgcaagtctc

SEQ ID NO: 23 - DNA, 20 bp, Artificial Sequence
  Description of Artificial Sequence Synthetic primer
ccctctttcacggtctcact

SEQ ID NO: 24 - DNA, 20 bp, Artificial Sequence
  Description of Artificial Sequence Synthetic primer
gggtagagcggctgtagaaa

SEQ ID NO: 25 - DNA, 20 bp, Artificial Sequence
  Description of Artificial Sequence Synthetic primer
gatttgtgggcctgaagaaa

SEQ ID NO: 26 - DNA, 20 bp, Artificial Sequence
  Description of Artificial Sequence Synthetic primer
ggaaaaaggggtttccagag

SEQ ID NO: 27 - DNA, 20 bp, Artificial Sequence
  Description of Artificial Sequence Synthetic primer
tcctgggaaggagacagaga

SEQ ID NO: 28 - DNA, 20 bp, Artificial Sequence
  Description of Artificial Sequence Synthetic primer
gaggtcaggcttcgctaatg

SEQ ID NO: 29 - DNA, 20 bp, Artificial Sequence
  Description of Artificial Sequence Synthetic primer
caagtggtttgtgcatcagg

SEQ ID NO: 30 - DNA, 20 bp, Artificial Sequence
  Description of Artificial Sequence Synthetic primer
gacggagaggaattcagacg

SEQ ID NO: 31 - DNA, 16 bp, Artificial Sequence
  Description of Artificial Sequence Synthetic primer
tgggcggcaccattgt

SEQ ID NO: 32 - DNA, 21 bp, Artificial Sequence
  Description of Artificial Sequence Synthetic primer
cataccttcccctgcaaatga